(19) United States
(12) Patent Application Publication
(10) Pub. No.: US 2026/0125763 A1
(43) Pub. Date: May 7, 2026

(54) METHODS AND SYSTEMS FOR CELL-FREE NUCLEIC ACID PROCESSING

(71) Applicant: Adela, Inc., Foster City, CA (US)

(72) Inventors: Daniel DINIZ DE CARVALHO, Toronto (CA); Abel LICON, Longmont, CO (US); Yulia NEWTON, Aptos, CA (US); Jun MIN, San Diego, CA (US); Iulia CIRLAN, Toronto (CA); Shu Yi Shen, Markham (CA); Felicia VINCELLI, Toronto (CA); Junjun ZHANG, Toronto (CA)

(21) Appl. No.: 19/354,332

(22) Filed: Oct. 9, 2025

Related U.S. Application Data
(63) Continuation of application No. PCT/US2024/024491, filed on Apr. 12, 2024.
(60) Provisional application No. 63/496,347, filed on Apr. 14, 2023.
(60) Provisional application No. 63/501,359, filed on May 10, 2023.
(60) Provisional application No. 63/511,441, filed on Jun. 30, 2023.
(60) Provisional application No. 63/517,327, filed on Aug. 2, 2023.
(60) Provisional application No. 63/588,120, filed on Oct. 5, 2023.
(60) Provisional application No. 63/591,732, filed on Oct. 19, 2023.
(60) Provisional application No. 63/594,365, filed on Oct. 30, 2023.
(60) Provisional application No. 63/602,156, filed on Nov. 22, 2023.
(60) Provisional application No. 63/549,294, filed on Feb. 2, 2024.
(60) Provisional application No. 63/571,139, filed on Mar. 28, 2024.

(51) Int. Cl. C12Q 1/6886

(52) U.S. Cl. CPC C12Q 1/6886; C12Q 2600/154

(57) ABSTRACT

Methods and systems for targeted detection of circulating tumor DNA (ctDNA) molecules are disclosed herein. In some cases, a molecular sequencing library depleted of methylated DNA can be generated and used to detect ctDNA in a cell-free DNA sample reliably at a lower sequencing depth and lower cost than existing methods.

### CROSS REFERENCE

This application is a continuation of International Application No. PCT/US2024/024491, filed Apr. 12, 2024, which claims the benefit of U.S. Provisional Application No. 63/496,347, filed Apr. 14, 2023, U.S. Provisional Application No. 63/501,359, filed May 10, 2023, U.S. Provisional Application No. 63/511,441, filed Jun. 30, 2023, U.S. Provisional Application No. 63/517,327, filed Aug. 2, 2023, U.S. Provisional Application No. 63/588,120 filed Oct. 5, 2023, U.S. Provisional Application No. 63/591,732, filed Oct. 19, 2023, U.S. Provisional Application No. 63/594,365, filed Oct. 30, 2023, U.S. Provisional Application No. 63/602,156, filed Nov. 22, 2023, U.S. Provisional Application No. 63/549,294, filed Feb. 2, 2024, and U.S. Provisional Application No. 63/571,139 filed Mar. 28, 2024, each of which are incorporated herein by reference in their entirety.

### BACKGROUND

Circulating tumor DNA (ctDNA) has increasingly demonstrated potential as a non-invasive, tumor-specific biomarker for routine clinical use. ctDNA is derived from tumor cells predominantly undergoing cell-death and released into circulation of various bodily fluids including blood. In most cancer patients, the majority of blood-derived cell-free DNA originates from healthy (e.g., non-cancerous) tissues. In addition, the fraction of ctDNA observed may range from <0.1% to 90% of total cell-free DNA at diagnosis depending on several factors including primary site of the tumor and disease burden. ctDNA provides non-invasive access to the tumor's molecular landscape and disease burden.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, provided herein is a method, comprising: (a) providing a plurality of nucleic acid molecules generated from a cell-free deoxynucleic acid (cfDNA) sample of a subject; (b) subjecting said plurality of nucleic acid molecules or derivatives thereof to sequencing to generate a plurality of sequencing reads; (c) computer processing said plurality of sequencing reads generate a methylation profile for said plurality of nucleic acid molecules; and (d) computer processing said methylation profile to determine that said subject has cancer at an area under the receiver operating characteristic curve (AUROC) of at least about 91%, wherein said cancer is low-shedding cancer. In some embodiments, said cfDNA sample comprises circulating tumor nucleic acid molecules derived from a low-shedding tumor. In some embodiments, said cancer is bladder cancer, breast cancer, endometrial cancer, prostate cancer, or renal cancer. In some embodiments, said cancer is endometrial cancer or prostate cancer.

In some aspects, provided herein, is a method, comprising: (a) providing a plurality of nucleic acid molecules generated from a cell-free deoxynucleic acid (cfDNA) sample of a subject; (b) subjecting said plurality of nucleic acid molecules or derivatives thereof to sequencing to generate a plurality of sequencing reads; (c) computer processing said plurality of sequencing reads generate a methylation profile for said plurality of nucleic acid molecules; and (d) computer processing said methylation profile to determine that said subject has cancer at an area under the receiver operating characteristic curve (AUROC) of at least about 94% and wherein said cancer is an early-stage cancer. In some embodiments, said cfDNA sample comprises circulating tumor nucleic acid molecules derived from an early-stage tumor. In some embodiments, said early-stage tumor is stage I tumor. In some embodiments, said early-stage tumor is stage II tumor. In some embodiments, said cancer is bladder cancer, breast cancer, colorectal cancer, endometrial cancer, esophageal cancer, head & neck cancer, hepatobiliary cancer, lung cancer, ovarian cancer, prostate cancer, or renal cancer. In some embodiments, said cancer is esophageal cancer, hepatobiliary cancer, or ovarian cancer.

In some aspects, provided herein, is a method, comprising: (a) providing a plurality of nucleic acid molecules generated from a cell-free deoxynucleic acid (cfDNA) sample of a subject; (b) subjecting said plurality of nucleic acid molecules or derivatives thereof to sequencing to generate a plurality of sequencing reads in absence of bisulfite conversion; (c) computer processing said plurality of sequencing reads generate a methylation profile for said plurality of nucleic acid molecules; and (d) computer processing said methylation profile to determine that said subject has cancer, wherein said cancer is endometrial cancer, esophageal cancer, hepatobiliary cancer, ovarian cancer, prostate cancer, bladder cancer, breast cancer, colorectal cancer, head and neck cancer, lung cancer, pancreatic cancer, or renal cancer. In some embodiments, said cfDNA sample comprises circulating tumor nucleic acid molecules derived from endometrial cancer. In some embodiments, computer processing said methylation profile to determine that said subject has endometrial cancer at an area under the receiver operating characteristic curve (AUROC) of at least about 90%. In some embodiments, said cfDNA sample comprises circulating tumor nucleic acid molecules derived from esophageal cancer. In some embodiments, computer processing said methylation profile to determine that said subject has esophageal cancer at an area under the receiver operating characteristic curve (AUROC) of at least about 99%. In some embodiments, said cfDNA sample comprises circulating tumor nucleic acid molecules derived from hepatobiliary cancer. In some embodiments, computer processing said methylation profile to determine that said subject has hepatobiliary cancer at an area under the receiver operating characteristic curve (AUROC) of at least about 99%. In some embodiments, said cfDNA sample comprises circulating tumor nucleic acid molecules derived from ovarian cancer. In some embodiments, computer processing said methylation profile to determine that said subject has ovarian cancer at an area under the receiver operating characteristic curve (AUROC) of at least about 97%. In some embodiments, said cfDNA sample comprises circulating tumor nucleic acid molecules derived from prostate cancer. In some embodiments, computer processing said methylation profile to determine that said subject has prostate cancer at an area under the receiver operating characteristic curve (AUROC) of at least about 89%. In some embodiments, said cfDNA sample comprises circulating tumor nucleic acid molecules derived from bladder cancer. In some embodiments, computer processing said methylation profile to determine that said subject has bladder cancer at an area under the receiver operating characteristic curve (AUROC) of at least about 95%. In some embodiments, said cfDNA sample comprises circulating tumor nucleic acid molecules derived from breast cancer. In some embodiments, computer processing said methylation profile to determine that said subject has breast cancer at an area under the receiver operating characteristic curve (AUROC) of at least about 92%. In some embodiments, said cfDNA sample comprises circulating tumor nucleic acid molecules derived from colorectal cancer. In some embodiments, computer processing said methylation profile to determine that said subject has colorectal cancer at an area under the receiver operating characteristic curve (AUROC) of at least about 98%. In some embodiments, said cfDNA sample comprises circulating tumor nucleic acid molecules derived from head and neck cancer. In some embodiments, computer processing said methylation profile to determine that said subject has head and neck cancer at an area under the receiver operating characteristic curve (AUROC) of at least about 96%. In some embodiments, said cfDNA sample comprises circulating tumor nucleic acid molecules derived from lung cancer. In some embodiments, computer processing said methylation profile to determine that said subject has lung cancer at an area under the receiver operating characteristic curve (AUROC) of at least about 96%. In some embodiments, said cfDNA sample comprises circulating tumor nucleic acid molecules derived from pancreatic cancer. In some embodiments, computer processing said methylation profile to determine that said subject has pancreatic cancer at an area under the receiver operating characteristic curve (AUROC) of at least about 99%. In some embodiments, said cfDNA sample comprises circulating tumor nucleic acid molecules derived from renal cancer. In some embodiments, computer processing said methylation profile to determine that said subject has renal cancer at an area under the receiver operating characteristic curve (AUROC) of at least about 91%.

In some embodiments, said method further comprises, prior to (b), adding a set of nucleic acid molecules that is not from said subject to said plurality of said nucleic acids molecules. In some embodiments, said methylation profile is genome wide. In some embodiments, said methylation profile comprise a whole methylome. In some embodiments, a method (e.g., step (d)) comprises a supervised machine learning method, wherein said supervised machine learning method is a regression, support vector machine, tree-based method, neural network, or nearest neighbor method. In some embodiments, a method (e.g., step (d)) comprises an unsupervised machine learning method, wherein said unsupervised machine learning method is clustering, neural network, principal component analysis, or matrix factorization. In some embodiments, said subject was previously treated for a cancer and was substantially free of said cancer, wherein (d) comprises determining said subject as having a recurrence of said cancer. In some embodiments, a method further comprises, prior to (b), adding an amount of filler DNA to said plurality of nucleic acid molecules or derivatives thereof. In some embodiments, said amount of filler DNA comprises double-stranded DNA. In some embodiments, said amount of filler DNA is from about 20 nanograms (ng) to about 100 ng. In some embodiments, at least a portion of said filler DNA is methylated. In some embodiments, between 10%-40% of said filler DNA is methylated, with a remainder being unmethylated filler DNA. In some embodiments, said method further comprises, prior to (b), contacting said cfDNA sample with a methylated nucleic acid capture reagent to generate said plurality of nucleic acid molecules, wherein said plurality of nucleic acids comprises one or more methylated regions. In some embodiments, said methylated nucleic capture agent comprises a binder and a solid substrate. In some embodiments, said methylated nucleic acid capture reagent is generated by coupling said binder to said solid substrate by incubating said binder with said solid substrate. In some embodiments, said coupling said binder to said solid substrate is performed prior to said contacting said cfDNA sample with a methylated nucleic acid capture reagent. In some embodiments, said solid substrate is a bead. In some embodiments, said solid substrate is a protein A bead. In some embodiments, said solid substrate is a magnetic solid substrate. In some embodiments, said binder comprises an antibody. In some embodiments, said binder is selected from a group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof. In some embodiments, said plurality of nucleic acids are enriched for one or more methylated regions at a specificity of at least about 99%. In some embodiments, said method further comprises, prior to (b), amplifying said plurality of nucleic acid molecules to generate amplicons, wherein (c) comprises sequencing said amplicons. In some embodiments, said amplifying is performed on said plurality of nucleic acids while said plurality of nucleic acids are bound to a solid support. In some embodiments, said amplifying comprises PCR amplification. In some embodiments, said PCR amplification comprises at least 13 or at least 14 cycles. In some embodiments, said method further comprises, prior to (b), contacting said plurality of nucleic acid molecules with one or more nucleic acid capture probes to enrich for one or more target sequences. In some embodiments, said one or more target sequences comprises one or more genes.

In some aspects, provided herein, is a method of processing a nucleic acid sample from a subject, said method comprising: (a) generating a nucleic acid sample mixture comprising a plurality of methylated nucleic acids from said subject and a plurality of filler nucleic acids, wherein said plurality of filler nucleic acids comprises at least one methylated nucleic acid molecule; (b) incubating (i) a methylation binding molecule with (ii) a solid substrate to form a methylated nucleic acid capture reagent; (c) capturing said methylated nucleic acid by adding said methylated nucleic acid capture reagent to said nucleic acid sample mixture to enrich said nucleic acid sample mixture for said plurality of methylated nucleic acids. In some embodiments, said method further comprises, subsequent to said capturing, amplifying said captured methylated nucleic acid to generate amplicons of said plurality of methylated nucleic acids. In some embodiments, said amplifying is performed while said plurality of methylated nucleic acids is bound to said methylated nucleic acid capture reagent. In some embodiments, said captured methylated nucleic acids are not subjected to an elution reaction prior to amplifying. In some embodiments, said method further comprises subjecting said plurality of methylated nucleic acids, or derivatives thereof, to a sequencing reaction. In some embodiments, said sequencing reaction is a sequencing by synthesis reaction. In some embodiments, said sequencing reaction does not comprise bisulfite sequencing. In some embodiments, said plurality of methylated nucleic acids comprises cell-free nucleic acids. In some embodiments, said solid substrate is a bead. In some embodiments, said solid substrate is a magnetic solid substrate. In some embodiments, said solid substrate comprises protein A. In some embodiments, said solid substrate comprises streptavidin. In some embodiments, said methylation binding molecule is an antibody. In some embodiments, said methylation binding molecule comprises biotin. In some embodiments, said methylation binding molecule binds to a methylated cytosine. In some embodiments, said method further comprises, prior to (a), obtaining said nucleic acids sample from said subject and performing one or more library preparation reactions on said nucleic acid sample. In some embodiments, said method further comprises, subsequent to said performing said one or more library preparation reactions, and prior to (a), incubating said nucleic acid sample with a plurality of magnetic beads that interact with nucleic acids. In some embodiments, said method further comprises, subsequent to incubating said nucleic acid sample with a plurality of magnetic beads that interact with nucleic acids, subjecting said sample to a magnetic capture to remove said plurality of magnetic beads that interact with nucleic acids. In some embodiments said method further comprises, performing an additional magnetic capture. In some embodiments said method further comprises, comprising subsequent to (a) and prior to (c), denaturing nucleic acids in said nucleic acid sample mixture. In some embodiments, said plurality of methylated nucleic acids in said nucleic acid sample mixture are enriched by at least 2 fold. In some embodiments, said plurality of methylated nucleic acids in said nucleic acid sample mixture are enriched by at least 100 fold. In some embodiments, said plurality of methylated nucleic acids in said nucleic acid sample mixture are enriched at a specificity of at least 99%. In some embodiments, said plurality of methylated nucleic acids in said nucleic acid sample mixture are enriched at a specificity of at a specificity of at least 99.5%. In some embodiments said method further comprises, contacting said plurality of methylated nucleic acids with one or more nucleic acid capture probes to enrich for one or more target sequences. In some embodiments, said one or more target sequences comprises one or more genes.

In some aspects, provided herein, is a method of processing a nucleic acid sample from a subject, said method comprising: (a) providing a nucleic acid sample comprising a plurality of methylated nucleic acids; (b) incubating (i) a methylation binding molecule with (ii) a solid substrate to form a methylated nucleic acid capture reagent; (c) capturing said plurality of methylated nucleic acids by adding said methylated nucleic acid capture reagent to said nucleic acid sample mixture to enrich said nucleic acid sample mixture for said plurality of methylated nucleic acids, wherein said plurality of methylated nucleic acids are enriched at a specificity of greater than 99%. In some embodiments said method further comprises, subsequent to said capturing, amplifying said captured methylated nucleic acids to generate amplicons of plurality of methylated nucleic acids. In some embodiments, said amplifying is performed while said plurality of methylated nucleic acids is bound to said methylated nucleic acid capture reagent. In some embodiments, said captured methylated nucleic acids are not subjected to an elution reaction prior to amplifying. In some embodiments, said method further comprises subjecting said plurality of methylated nucleic acids, or derivatives thereof, to a sequencing reaction. In some embodiments, said sequencing reaction is a sequencing by synthesis reaction. In some embodiments, said sequencing reaction does not comprise bisulfite sequencing. In some embodiments, said plurality of methylated nucleic acids comprises cell-free nucleic acids. In some embodiments, said solid substrate is a bead. In some embodiments, said solid substrate is a magnetic solid substrate. In some embodiments, said solid substrate comprises protein A. In some embodiments, said solid substrate comprises streptavidin. In some embodiments, said methylation binding molecule is an antibody. In some embodiments, said methylation binding molecule comprises biotin. In some embodiments, said methylation binding molecule binds to a methylated cytosine. In some embodiments said method further comprises, prior to (c), performing one or more library preparation reactions on said methylated nucleic acid. In some embodiments said method further comprises, subsequent to said performing said one or more library preparation reactions, and prior to (c), incubating said nucleic acid sample with a plurality of magnetic beads that interact with nucleic acids. In some embodiments said method further comprises, subsequent to incubating said nucleic acid sample with a plurality of magnetic beads that interact with nucleic acids, subjecting said sample to a magnetic capture to remove said plurality of magnetic beads that interact with nucleic acids. In some embodiments said method further comprises, performing an additional magnetic capture. In some embodiments said method further comprises, subsequent to (a) and prior to (c), denaturing nucleic acids in said nucleic acid sample. In some embodiments, said plurality of methylated nucleic acids in said nucleic acid sample are enriched by at least 2 fold. In some embodiments, said plurality of methylated nucleic acids in said nucleic acid sample are enriched by at least 100 fold. In some embodiments, said plurality of methylated nucleic acids in said nucleic acid sample are enriched at a specificity of at least 99.5%. In some embodiments said method further comprises, contacting said plurality of methylated nucleic acids with one or more nucleic acid capture probes to enrich for one or more target sequences. In some embodiments, said one or more target sequences comprises one or more genes.

In some aspects, provided herein, is a method of processing a nucleic acid sample from a subject, said method comprising: (a) providing a nucleic acid sample comprising a plurality of methylated nucleic acids; (b) incubating (i) a methylation binding molecule with (ii) a solid substrate to form a methylated nucleic acid capture reagent; (c) capturing said methylated nucleic acid by adding said methylated nucleic acid capture reagent to said nucleic acid sample, thereby generating solid substrate bound methylated nucleic acid; (d) amplifying said solid substrate bound methylated nucleic acid to generate amplicons of said plurality of methylated nucleic acid. In some embodiments, said amplifying is performed while said plurality of methylated nucleic acid is bound to said methylated nucleic acid capture reagent. In some embodiments, said capture methylated nucleic acid are not subjected to an elution reaction prior to amplifying. In some embodiments, said method further comprises subjecting said amplicons of methylated nucleic acid to a sequencing reaction. In some embodiments, said sequencing reaction is a sequencing by synthesis reaction. In some embodiments, said sequencing reaction does not comprise bisulfite sequencing. In some embodiments, said plurality of methylated nucleic acids comprises cell-free nucleic acids. In some embodiments, said solid substrate is a bead. In some embodiments, said solid substrate is a magnetic solid substrate. In some embodiments, said solid substrate comprises protein A. In some embodiments, said solid substrate comprises streptavidin. In some embodiments, said methylation binding molecule is an antibody. In some embodiments, said methylation binding molecule comprises biotin. In some embodiments, said methylation binding molecule binds to a methylated cytosine. In some embodiments, said method further comprises, prior to (c), performing one or more library preparation reactions on said methylated nucleic acid. In some embodiments, said method further comprises, subsequent to said performing said one or more library preparation reactions, and prior to (c), incubating said nucleic acid sample with a plurality of magnetic beads that interact with nucleic acids. In some embodiments, said method further comprises, subsequent to incubating said nucleic acid sample with a plurality of magnetic beads that interact with nucleic acids, subjecting said sample to a magnetic capture to remove said plurality of magnetic beads that interact with nucleic acids. In some embodiments, said method further comprises, performing an additional magnetic capture. In some embodiments, said method further comprises, subsequent to (a) and prior to (c), denaturing nucleic acids in said nucleic acid sample. In some embodiments, said plurality of methylated nucleic acids in said nucleic acid sample are enriched by at least 2 fold. In some embodiments, said plurality of methylated nucleic acids in said nucleic acid sample are enriched by at least 100 fold. In some embodiments, said plurality of methylated nucleic acids in said nucleic acid sample are enriched at a specificity of at least 99%. In some embodiments, said plurality of methylated nucleic acids in said nucleic acid sample are enriched at a specificity of at least 99.5%. In some embodiments, said method further comprises, contacting said plurality of methylated nucleic acids with one or more nucleic acid capture probes to enrich for one or more target sequences. In some embodiments, said one or more target sequences comprises one or more genes.

In some aspects, provided herein, is a method of processing a nucleic acid sample from a subject, said method comprising: (a) generating a nucleic acids sample mixture comprising a plurality of methylated nucleic acids from said subject and a plurality of filler nucleic acids, wherein said a plurality of filler nucleic acids comprises at least one methylated nucleic acid molecule; (b) capturing said methylated nucleic acid by adding a capture reagent that comprises a solid substrate to said nucleic acid sample mixture, thereby generating solid substrate bound methylated nucleic acid; and (c) amplifying said solid substrate bound methylated nucleic acid to generate amplicons of methylated nucleic acid. In some embodiments, said capture methylated nucleic acid are not subjected to an elution reaction prior to amplifying. In some embodiments, said method further comprises, subjecting said amplicons of methylated nucleic acid to a sequencing reaction. In some embodiments, said sequencing reaction is a sequencing by synthesis reaction. In some embodiments, said sequencing reaction does not comprise bisulfite sequencing. In some embodiments, said plurality of methylated nucleic acids comprises cell-free nucleic acids. In some embodiments, said solid substrate is a bead. In some embodiments, said solid substrate is a magnetic solid substrate. In some embodiments, said solid substrate comprises protein A. In some embodiments, said solid substrate comprises streptavidin. In some embodiments, said capture reagent is a methylated nucleic acids capture agent. In some embodiments, said methylated nucleic acids capture agent comprises a methylation binding molecule attached to said solid substrate. In some embodiments, said methylation binding molecule is an antibody. In some embodiments, said methylation binding molecule comprises biotin. In some embodiments, said methylation binding molecule binds to a methylated cytosine. In some embodiments, said method further comprises, prior to (a), obtaining said nucleic acids sample from said subject and performing one or more library preparation reactions on said nucleic acids. In some embodiments, said method further comprises, subsequent to said performing said one or more library preparation reactions, and prior to (a), incubating said nucleic acid sample with a plurality of magnetic beads that interact with nucleic acids. In some embodiments, said method further comprises, subsequent to incubating said nucleic acid sample with a plurality of magnetic beads that interact with nucleic acids, subjecting said sample to a magnetic capture to remove said plurality of magnetic beads that interact with nucleic acids. In some embodiments, said method further comprises, performing an additional magnetic capture. In some embodiments, said method further comprises, subsequent to (a) and prior to (b), denaturing nucleic acids in said nucleic acid sample mixture. In some embodiments, said plurality of methylated nucleic acids in said nucleic acid sample mixture are enriched by at least 2 fold. In some embodiments, said plurality of methylated nucleic acids in said nucleic acid sample mixture are enriched by at least 100 fold. In some embodiments, said plurality of methylated nucleic acids in said nucleic acid sample mixture are enriched at a specificity of at least 99%. In some embodiments, said plurality of methylated nucleic acids in said nucleic acid sample mixture are enriched at a specificity of at least 99.5%. In some embodiments, said method further comprises, contacting said plurality of methylated nucleic acids with one or more nucleic acid capture probes to enrich for one or more target sequences. In some embodiments, said one or more target sequences comprises one or more genes.

In some aspects, provided herein, is a method comprising: (a) obtaining a first nucleic acid molecules from a cell free sample of a subject; (b) generating a second set of nucleic acid molecules from the first set of nucleic acid molecules or a derivative thereof, wherein said second set of nucleic acid molecules is enriched for methylation level relative to said first set of nucleic acid molecules; (c) enriching said second set of nucleic acid molecules or a derivative thereof for one or more targets to yield a third set of nucleic acid molecules; and (d) sequencing said third set of nucleic acid molecules or a derivative thereof. In some embodiments, said enriching comprises contacting said second set of nucleic acid molecules or derivatives thereof with one or more nucleic acid capture probes. In some embodiments, said generating comprise contacting said first set of nucleic acids molecules or said derivative thereof with a methylated nucleic acid capture reagent. In some embodiments, said methylated nucleic acid capture reagent is formed by incubating a methylation binding molecule with (ii) a solid substrate. In some embodiments, said solid substrate is a bead. In some embodiments, said solid substrate is a magnetic solid substrate. In some embodiments, said solid substrate comprises protein A. In some embodiments, said solid substrate comprises streptavidin. In some embodiments, said methylation binding molecule is an antibody. In some embodiments, said methylation binding molecule comprises biotin. In some embodiments, said methylation binding molecule binds to a methylated cytosine. In some embodiments, the method further comprises amplifying said second set of molecules. In some embodiments, said amplifying is performed while a subset of said first set of nucleic acids are bound to said methylated nucleic acid capture reagent. In some embodiments, said sequencing is a sequencing by synthesis reaction. In some embodiments, said sequencing does not comprise bisulfite sequencing. In some embodiments, prior to said sequencing, performing one or more library preparation reactions on said third set of nucleic acid molecules. In some embodiments, subsequent to said performing said one or more library preparation reactions, and prior to said sequencing, incubating said third set of nucleic acid molecules with a plurality of magnetic beads that interact with nucleic acids. In some embodiments, said method further comprises subsequent to incubating said nucleic acid sample with a plurality of magnetic beads that interact with nucleic acids, subjecting said sample to a magnetic capture to remove said plurality of magnetic beads that interact with nucleic acids. In some embodiments, said method further comprises performing an additional magnetic capture. In some embodiments, said method further comprises, prior to (b), adding an amount of filler DNA to said first nucleic acid molecules.

### BRIEF DESCRIPTION OF FIGURES

These and other features of the preferred embodiments of the invention will become more apparent in the following detailed description in which reference is made to the appended drawings wherein:

FIG. 1 shows a diagram illustrating a process for collecting flow-through of unmethylated/hypomethylated DNA fragments

FIG. 2 shows a schematic of a computer system, in accordance with embodiments of the present disclosure.

FIG. 3 illustrates receiver operating characteristic (ROC) curve for overall cohort of 12 cancers.

FIGS. 4A-4L illustrate multi-cancer early detection by cancer type. FIG. 4A shows ROC curve (95% confidence intervals) for bladder cancer with area under the ROC curve (AUC) for all stages, stage I, stage II, stage III, and stage IV. FIG. 4B shows ROC curve (95% confidence intervals) for breast cancer with AUC for all stages, stage I, stage II, stage III, and stage IV. FIG. 4C shows ROC curve (95% confidence intervals) for colorectal cancer with AUC for all stages, stage I, stage II, stage III, and stage IV. FIG. 4D shows ROC curve (95% confidence intervals) for endometrial cancer with AUC for all stages, stage I, stage II, stage III, and stage IV. FIG. 4E shows ROC curve (95% confidence intervals) for esophageal cancer with AUC for all stages, stage I, stage II, stage III, and stage IV. FIG. 4F shows ROC curve (95% confidence intervals) for head and neck cancer with AUC for all stages, stage I, stage II, stage III, and stage IV. FIG. 4G shows ROC curve (95% confidence intervals) for hepatobiliary cancer with AUC for all stages, stage I, stage II, stage III, and stage IV. FIG. 4H shows ROC curve (95% confidence intervals) for lung cancer with AUC for all stages, stage I, stage II, stage III, and stage IV. FIG. 4I shows ROC curve (95% confidence intervals) for ovarian cancer with AUC for all stages, stage I, stage II, stage III, and stage IV. FIG. 4J shows ROC curve (95% confidence intervals) for pancreatic cancer with AUC for all stages, stage I, stage II, stage III, and stage IV. FIG. 4K shows ROC curve (95% confidence intervals) for prostate cancer with AUC for all stages, stage I, stage II, stage III, and stage IV. FIG. 4L shows ROC curve (95% confidence intervals) for renal cancer with AUC for all stages, stage I, stage II, stage III, and stage IV.

FIG. 5A-5B illustrate ctDNA quantification and prognostic prediction in renal cancer. FIG. 5A shows ctDNA quantification scores for renal cancer generated based on average normalized counts of reduced-size fragments to identify cancer-associated methylation across 2027 regions and adjusted for methylation specificity. A ctDNA quantification score threshold was set such that 95% of samples without recurrence or progression fell below the threshold. FIG. 5B shows a Kaplan-Meier plot of renal cancer recurrence/progression-free survival probability over time.

FIG. 6A-6B illustrate ctDNA quantification and prognostic prediction in head and neck cancer. FIG. 6A shows ctDNA quantification scores for head and neck cancers cancer generated based on average normalized counts of reduced-size fragments to identify cancer-associated methylation across 2027 regions and adjusted for methylation specificity. A ctDNA quantification score threshold was set such that 95% of samples without recurrence or progression fell below the threshold. FIG. 6B shows a Kaplan-Meier plot of head and neck cancer recurrence/progression-free survival probability over time.

FIG. 7 shows a Kaplan-Meier plot depicting event-free survival over time of individuals with head and neck cancer, stratified by ctDNA quantification.

FIG. 8 shows the age of individuals with Renal Cell Carcinoma (RCC) at the time of sample collection.

FIG. 9 shows a Kaplan-Meier plot depicting event-free survival over time of individuals with RCC of all stages.

FIG. 10 shows a Kaplan-Meier plot depicting event-free survival over time of individuals with RCC of stages I-III.

FIG. 11 shows a Kaplan-Meier plot depicting recurrence-free survival over time of individuals with early-stage non-small cell lung cancer (NSCLC).

FIG. 12 shows a schematic of patient treatment and blood collection, sample processing, and genome wide methylation enrichment platform.

FIG. 13A-13B illustrate a Kaplan-Meier plot depicting recurrence-free survival probability over time of individuals who predict positive or negative for head and neck cancer based on ctDNA. FIG. 13A shows a Kaplan-Meier plot depicting recurrence-free survival probability over time of individuals who predict positive or negative for head and neck cancer based on ctDNA at the landmark timepoint. FIG. 13B shows a Kaplan-Meier plot depicting recurrence-free survival probability over time of individuals who predict positive or negative for head and neck cancer based on ctDNA, longitudinally.

FIG. 14 shows estimated ctDNA quantification from pre-treatment to post-treatment of head and neck cancer patients with non-recurrence or with recurrence.

FIG. 15 shows representative case studies of ctDNA kinetics in individual patients before and after curative intent treatment.

FIG. 16 shows an example of a schematic overview of the whole genome methylation enrichment platform.

FIG. 17 shows the experimental design for evaluating limit of detection of the whole genome methylation enrichment platform.

FIG. 18 shows the methylation specificity distribution and unique molecules distribution of all non-cancer and contrived cancer samples processed by the whole genome methylation enrichment platform disclosed herein.

FIG. 19 shows the ctDNA methylation score of different cfDNA sources titrated into pooled non-cancer donor-derived cfDNA in a titration series targeting less than 1% ctDNA levels.

FIG. 20 shows the experimental design for evaluating precision of the whole genome methylation enrichment platform disclosed herein.

FIG. 21 shows the agreement and variability using ctDNA methylation score obtained for varying levels of ctDNA that were subjected to the whole genome methylation enrichment platform disclosed herein.

FIG. 22 shows the variance components percentage (CV %) of varying operators, sequencing runs, and antibody reagent lots calculated for varying levels of ctDNA that were subjected to the whole genome methylation enrichment platform disclosed herein.

FIG. 23 shows genomic contamination of cell-free DNA.

FIG. 24 shows 0 CpG counts for low and high binding specificity samples.

FIG. 25 shows four different workflows for processing plasma-derived cell-free DNA using a whole methylome enrichment platform.

FIG. 26 shows methylation specificity for different workflows outlined in FIG. 25.

FIG. 27 shows the improvement in the Limit of Detection in the cancer methylome approach versus the whole methylome approach as a fold change.

FIG. 28 shows a workflow for processing plasma-derived cell-free DNA using a whole methylome enrichment platform followed by capturing of target regions with probes.

### DETAILED DESCRIPTION

The present disclosure provides methods and systems for the processing and analysis of nucleic acids present in biological samples, which can be useful in determining a risk or likelihood of a subject having cancer or a tumor with high sensitivity and/or high specificity. Methods and systems provided herein can comprise the creation of hypermethylated cell-free nucleic acid molecules which can be processed to differentiate between, for example, cancerous and non-cancerous tissue in circulating free DNA (cfDNA).

For example, the use and analysis of hypermethylated nucleic acids can allow for highly sensitive and highly specific detection and/or characterization of circulating tumor DNA (ctDNA) in a fluid sample (e.g., a blood sample) obtained from a subject. In some cases, the use and analysis of hypermethylated nucleic acids can allow for increased sensitivity, specificity, and/or efficiency in the determination of a subject's risk of having or having a risk of developing a tumor or cancer. Methods for detecting ctDNA with increased sensitivity are needed, especially in subjects with lower abundance of ctDNA.

### Definitions

The term “subject” as used herein generally refers to any member of the animal kingdom (e.g., human, non-human primate, mouse, rate, bovine, ovine, equine, canine, feline, rabbit, horse or goat). Thus, the methods and described herein are applicable to both human and veterinary disease and animal models. Preferred subjects are “patients,” i.e., living humans that are being investigated to determine whether treatment or medical care is needed for a disease or condition; or that are receiving medical care for a disease or condition (e.g., cancer).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The term “nucleic acid” used herein generally refers to a polynucleotide comprising two or more nucleotides, i.e., a polymeric form of nucleotides of any length, either deoxyribonucleotides (dNTPs) or ribonucleotides (rNTPs), or analogs thereof. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, at least about 90%, at least about 95%, or at least about 99%, identity to the nucleotide sequence of the original nucleic acid.

Cell-free methylated DNA is DNA that can be one or more nucleic acid molecules circulating freely in the blood stream. In some cases, cell-free methylated DNA can be methylated at various regions of the DNA. Samples, for example, plasma samples may be taken to analyze cell-free methylated DNA. Studies reveal that much of the circulating nucleic acids in blood arise from necrotic or apoptotic cells and greatly elevated levels of nucleic acids from apoptosis is observed in diseases such as cancer. Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. For example, a quantitative assay for low levels of circulating tumor DNA in total circulating DNA may serve as a better marker for detecting the relapse of colorectal cancer compared with carcinoembryonic antigen, the standard biomarker used clinically. Cell-free DNA (e.g., circulating cfDNA) may comprise circulating tumor DNA (ctDNA).

As used herein, “library preparation” generally includes one or more of list end-repair, A-tailing, adapter ligation, or any other preparation performed on the cell free DNA to permit subsequent sequencing of DNA.

As used herein, “supplemental processed DNA” (e.g., “filler DNA”) may be noncoding DNA or it may consist of amplicons.

In some embodiments, the fragment length metric is fragment length. In some preferable embodiments, the subject cell-free methylated DNA is limited to fragments having a length of <170 bp, <165 bp, <160 bp, <155 bp, <150 bp, <145 bp, <140 bp, <135 bp, <130 bp, <125 bp, <120 bp, <115 bp, <110 bp, <105 bp, or <100 bp. In other preferable embodiments, the subject cell-free methylated DNA is limited to fragments having a length of between about 100-about 150 bp, 110-140 bp, or 120-130 bp.

In some embodiments, the fragment length metric is the fragment length distribution of the subject cell-free methylated DNA. In some preferable embodiments, the subject cell-free methylated DNA is limited to fragments within the bottom 50^{th}, 45^{th}, 40^{th}, 35^{th}, 30^{th}, 25^{th}, 20^{th}, 15^{th}, or 10^{th} percentile based on length.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### DNA Methylation

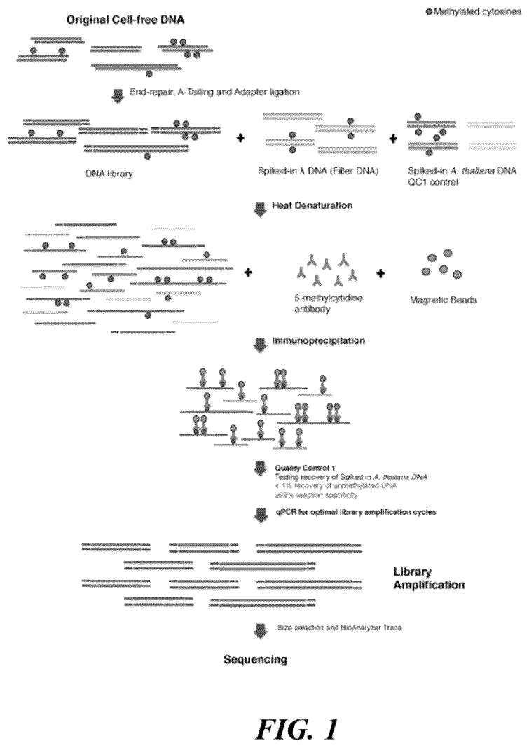

Cell-free DNA (cfDNA) may be processed and enriched for methylated cfDNA using any one of the methods disclosed herein. As shown in FIG. 1, cfDNA may undergo end-pair, A-tailing, adaptor ligation or other preparation thereof to produce a library of DNAs for downstream sequencing. The library may be combined with supplemental processed DNA (e.g., filler DNA) and/or spiked-in DNAs to produce a sample mixture, before heat denaturation and immunoprecipitation to enrich for methylated cfDNA. Immunoprecipitation can comprise combining the sample mixture to any one of the binders disclosed herein and a solid substrate (e.g., a plurality of magnetic beads). In some cases, immunoprecipitation can comprise combining the sample mixture with a methylated nucleic acid capture reagent, wherein the methylated nucleic acid capture reagent comprises an incubated mixture of a binder and a solid substrate. Upon enrichment of methylated cfDNA, the methylated cfDNA may undergo amplification before being subjected to sequencing (e.g., Illumina sequencing reaction).

Cell-free DNA (cfDNA), which can be present in biological samples that can be collected non-invasively (e.g., blood, urine, saliva, cerebrospinal fluid (CSF), etc.), can be a heterogeneous population comprising both cfDNA derived from healthy tissues and cfDNA derived from tumor or cancer cells (e.g., circulating tumor DNA (ctDNA)). Cancer development can be associated with focal gain of 5′ methylcytosines (5mC), for instance, at cytosine-phosphate-guanine (CpG) islands and CpG island shores. Cancer development can also be associated with global (e.g., genome-wide) cytosine demethylation (e.g., global loss of 5mC). In some cases, ctDNA can be distinguished from cfDNA molecules derived from healthy tissue (e.g., non-tumor and/or non-cancer tissue) by the methylation level (e.g., the percentage of nucleotide residues that are methylated) of the nucleic acid molecules. In some cases, nucleic acid molecules of or derived from tumor tissue and/or cancer tissue can be hypomethylated (e.g., can comprise a lower level of methylation, for instance, wherein there are fewer methylated nucleotide residues and/or a lower percentage of methylated nucleotide residues) compared to nucleic acid molecules of or derived from healthy tissue (e.g., nucleic acid molecules of or derived from healthy tissue that consist of or comprise nucleotide sequences corresponding to the same region(s) of the genome of the subject). For example, tumor-derived nucleic acid molecules (e.g., ctDNA molecules) can comprise one or more regions having fewer methylated nucleotide residues than nucleic acid molecules (e.g., cfDNA molecules) derived from healthy tissues (e.g., non-tumor and/or non-cancer tissues) in the same biological sample. In some cases, nucleic acid molecules of or derived from tumor tissue and/or cancer tissue can be hypermethylated (e.g., can comprise a higher level of methylation, for instance, wherein there are a greater number of methylated nucleotide residues and/or a higher percentage of methylated nucleotide residues) compared to nucleic acid molecules of or derived from healthy tissue (e.g., nucleic acid molecules of or derived from healthy tissue that consist of or comprise nucleotide sequences corresponding to the same region(s) of the genome of the subject). For example, tumor-derived nucleic acid molecules (e.g., ctDNA molecules) can comprise one or more regions having a greater number of methylated nucleotide residues than nucleic acid molecules (e.g., cfDNA molecules) derived from healthy tissues (e.g., non-tumor and/or non-cancer tissues) in the same biological sample. In some cases, all or a portion of a tumor-derived fraction of a plurality of cell-free DNA molecules (e.g., ctDNA) can be distinguished from cfDNA molecules derived from healthy tissue by one or more biophysical properties (e.g., the length of the cfDNA molecules or the presence of stereotypical 5′ and 3′ end sequence motifs) and/or one or more fragmentomics patterns. For instance, ctDNA molecules can have shorter nucleic acid lengths than cfDNA molecules derived from healthy tissues. In some cases, ctDNA molecules may comprise stereotypical 5′ and 3′ end motifs. In some cases, one or more of these distinguishing features may be used to deplete a population of nucleic acid molecules of cfDNA derived from healthy tissue and/or to enrich a population of nucleic acid molecules for ctDNA. ctDNA typically has shorter fragment length compared to cfDNA derived from a healthy tissue.

Nucleic acid molecules derived from tumor or cancer cells or tissue (e.g., ctDNA) may be present in a biological sample (and/or a population of nucleic acids derived from the biological sample) in substantially lower quantities than nucleic acid molecules (e.g., cfDNA) derived from healthy tissue. It can be difficult to detect or sequence (e.g., determine a sequence identity of) ctDNA present in a plurality of nucleic acid molecules (e.g., cfDNA) in or derived from a biological sample, for instance, because they are present in the sample in lower quantities relative to cfDNA derived from healthy tissue (e.g., which may require using a greater amount of potentially scarce biological sample and/or which may require significantly higher sequencing depth).

Depletion (e.g., removal) of all or a portion of the population of methylated DNA molecules (e.g., molecules having increased nucleotide methylation levels throughout or in a subset of the regions of the genome represented by the plurality of nucleic acid molecules of a biological sample) from a plurality of nucleic acid molecules (e.g., a plurality of cell-free nucleic acid molecules, or amplicons thereof, comprising a biological sample) may yield a remainder population of the plurality of nucleic acids of the biological sample that may be useful for determining a presence and/or sequence identity of ctDNA molecules in the biological sample. Typically, depletion/removing may be performed by using a binder specific for methylated DNA molecules to pull them down. The pull-down is typically collected and the flow-through containing the unmethylated/hypomethylated DNA molecules is discarded. The current disclosure provides for the first time methods and systems to collect such flow-through containing unmethylated/hypomethylated DNA molecules and to generate sequencing library using methylated/hypomethylated DNA molecules or derivatives thereof.

In some cases, a depleted sequencing library of methods, systems, compositions, and kits disclosed herein may consist of or can be comprised of such a remainder population of nucleic acid molecules. In some cases, it may be sufficient to deplete a plurality of nucleic acids (e.g., cfDNA molecules or amplicons thereof derived from a biological sample) of nucleic acid molecules methylated in one or more specific regions of the genomic sequence of the nucleic acid molecules (e.g., CpG islands, CpG island shores, or repetitive sequences of the genome, such as long interspersed nuclear elements (LINEs), short interspersed nuclear elements (SINEs), or LTRs (long terminal repeats)) to achieve increased sensitivity and/or increased specificity in assays for determining the presence or absence or the sequence identity of ctDNA molecules in the plurality. In some cases, a plurality of nucleic acids (e.g., cfDNA molecules or amplicons thereof derived from a biological sample) may be subjected to genome-wide depletion of nucleic acid molecules methylated in one or more specific regions of the genomic sequence of the nucleic acid molecules (e.g., CpG islands, CpG island shores, or repetitive sequences of the genome, such as long interspersed nuclear elements (LINEs), short interspersed nuclear elements (SINEs), or LTRs (long terminal repeats)) to achieve increased sensitivity and/or increased specificity in assays for determining the presence or absence or the sequence identity of ctDNA molecules in the plurality. In some cases, a remainder population (e.g., a plurality of nucleic acid fragments useful in the creation of a depleted library) can be deprived of CpG genomic islands. In some cases, a remainder population (e.g., a plurality of nucleic acid fragments useful in the creation of a depleted library) can comprise one or more of: long interspersed nuclear elements (LINEs), short interspersed nuclear elements (SINEs), or long terminal repeat (LTR) elements.

Enrichment of all or a portion of the population of methylated DNA molecules (e.g., molecules having increased nucleotide methylation levels throughout or in a subset of the regions of the genome represented by the plurality of nucleic acid molecules of a biological sample) from a plurality of nucleic acid molecules (e.g., a plurality of cell-free nucleic acid molecules, or amplicons thereof, comprising a biological sample) may yield a population of the plurality of nucleic acids of the biological sample that may be useful for determining a presence and/or sequence identity of ctDNA molecules in the biological sample. Enrichment may be performed by using a binder specific for methylated DNA molecules to pull them down. The pull-down can be collected and the flow-through containing the unmethylated/hypomethylated DNA molecules can be discarded or alternatively be collected (e.g., used to generate a depletion library as described in this disclosure). The enriched fraction may be then subjected to sequencing.

Depletion or enrichment of all or a portion of the methylated nucleic acid molecules of a plurality of nucleic acid molecules of a biological sample may comprise contacting the methylated nucleic acid molecules with a binder (e.g., an affinity molecule, such as an antibody or a protein, specific to methylated nucleotide residues). For example, creation of a sequencing library can comprise contacting a plurality of nucleic acid molecules (e.g., cfDNA molecules) or amplicons thereof with a binder selective for a methylated region of nucleic acid molecules (e.g., a methylcytosine binder (MBD), such as an MBD-Fc fusion protein). In some cases, a binder may be specific to one or more methylated nucleotide species (e.g., 5-methylcytosine (5mC)). Cell-free Methylated DNA Immunoprecipitation sequencing (cfMeDIP-seq), a genome-wide molecular profiling technique, can enrich for methylated cfDNA fragments through use of a binder, such as an anti-5-methylcytosine (anti-5mC) antibody or methyl-CpG-binding domain (MBD) protein (e.g., MBD-Fc fusion proteins). As described herein, cfMeDIP-seq can comprise a portion of methods and systems for depleting a cfDNA sample of methylated DNA fragments, leaving behind hypomethylated or unmethylated cfDNA fragments, such as ctDNA. Thus, the identification of hypomethylated or unmethylated cell-free DNA within a clinical sample may be useful in determining the presence of a tumor or cancer in a subject.

In some cases, depletion of a plurality of nucleic acid molecules (e.g., in the creation of a depleted sequencing library and/or the determination of a presence or sequence identity of a nucleic acid molecule) may comprise removing one or more nucleic acid molecules having a methylation level above a threshold methylation level (e.g., wherein the one or more removed nucleic acid molecules are hypermethylated, for instance, relative to one or more nucleic acid molecules not removed during depletion). In some cases, enrichment of a plurality of nucleic acid molecules (e.g., in the creation of a enriched sequencing library and/or the determination of a presence or sequence identity of a nucleic acid molecule) may comprise removing one or more nucleic acid molecules having a methylation level below a threshold methylation level (e.g., wherein the one or more removed nucleic acid molecules are hypomethylated, for instance, relative to one or more nucleic acid molecules not enriched, or non-methylated). In some cases, a methylation level of a particular nucleic acid fragments (e.g., DNA fragments) may be considered to reach the threshold methylation level when a binder with a sufficient specificity for methylated cytosines is able to bind to the particular nucleic acid fragments either with or without using filler DNA as described here. In some cases, a methylation level of particular nucleic acid fragments (e.g., DNA fragments) may be considered to be below the threshold methylation level when a binder with a sufficient specificity for methylated cytosines is not able to bind to the particular nucleic acid fragments either with or without using filler DNA as described here. In some cases, depletion of a plurality of nucleic acid molecules (e.g., in the creation of a depleted sequencing library and/or the determination of a presence or sequence of a nucleic acid molecule) results in (e.g., provides) a remainder population of the plurality of nucleic acid molecules, wherein the remainder of the plurality of nucleic acid molecules comprises (or, in some cases, consists of) nucleic acid molecules having a methylation level below the threshold methylation level (e.g., wherein the remainder population is hypomethylated/unmethylated relative to one or more nucleic acid molecules removed from the plurality of nucleic acid molecules during depletion). A methylation level may be calculated as a percentage of hypermethylated nucleic acid fragments compared to all the nucleic acid fragments contained in a sample. In some cases, a threshold methylation level can be from 0.1% to 1%, 1% to 5%, 5% to 10%, 10% to 15%, 15% to 20%, 20% to 25%, 25% to 30%, 30% to 35%, 35% to 40%, 40% to 45%, 45% to 50%, 50% to 55%, 55% to 60%, 65% to 70%, 70% to 75%, 75% to 80%, 80% to 85%, 85% to 90%, 95% to 100%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In some cases, a first plurality of nucleic acid molecules (e.g., comprising nucleic acid molecules, such as cfDNA, from a biological sample of a subject) may be combined (e.g., mixed) with a second plurality of nucleic acid molecules (e.g., wherein the second plurality of nucleic acid molecules is not from the subject from whom the biological sample was taken), for instance, as shown in FIG. 1. In some cases, the second plurality of nucleic acid molecules comprises supplemental processed DNA (e.g., comprising 1 DNA). In some cases, each of the second plurality of nucleic acid molecules does not align to a human genome.

In some cases, a method or system disclosed herein may comprise determining or identifying a sequence of all or a portion of a depleted nucleic acid molecule population (e.g., remainder population of a plurality of nucleic acid fragments of a biological sample after pulling down hypermethylated nucleic acid fragments), for example, using a sequencer. In some cases, a remainder population of nucleic acid molecules may be purified (e.g., after library creation) to yield a plurality of purified nucleic acid molecules, for example, prior to or as part of a process of determining or identifying a sequence of all or a portion of the depleted nucleic acid molecule population. In some cases, all or a portion of the plurality of purified nucleic acid molecules may be amplified (e.g., via polymerase chain reaction), for instance, prior to or as part of a process of determining or identifying a sequence of all or a portion of the depleted nucleic acid molecule population. In some cases, a population of amplified nucleic acid molecules or a derivative thereof (e.g., comprising amplicons of all or a portion of the plurality of purified nucleic acid molecules) may be subjected to sequencing (e.g., for the determination and/or identification of a sequence of the nucleic acid molecules). In some cases, the sequencing may be achieved using a sequencer, as described herein. In some cases, a sequence of a plurality of nucleic acid molecules of a biological sample (or a derivative thereof) may be identified or determined using an array or polymerase chain reaction. In some cases, the presence of a tumor-derived nucleic acid molecule may be determined by calculating a sum of reads per kilobase per million (RPKM) for a region of the genome (e.g., all or a portion of the genome, such as just CpG islands or just CpG island shores). In some cases, the presence of a tumor-derived nucleic acid molecule may be indicated when a depleted sequencing library (e.g., comprising a remainder population of nucleic acids) is observed to have a low sum of RPKMs, e.g., lower than 70,000, lower than 60,000, lower than 50,000, lower than 40,000, or lower than 30,000 across one or more regions of interest (e.g., CpG islands or CpG island shores).

### Supplemental Processed DNA (Filler DNA)

In some cases, supplemental processed DNA (e.g., filler DNA, filler nucleic acids) may be added to a first plurality of nucleic acids (e.g., a plurality of nucleic acids from a biological sample, which may comprise cfDNA from healthy tissue and/or cfDNA from tumor tissue, such as ctDNA). In some cases, addition of supplemental processed DNA (e.g., a second plurality of nucleic acid molecules) to a first plurality of nucleic acid molecules can increase the specificity and/or sensitivity of a method, system, or kit described herein, for instance, with respect to the detection and/or identification of a nucleic acid sequence of the first plurality of nucleic acid molecules. In some cases, addition of supplemental processed DNA (e.g., a second plurality of nucleic acid molecules) to a first plurality of nucleic acid molecules may increase the rate of depletion of a methylated region of a nucleic acid sequence, e.g., during the practice of some embodiments of methods and systems described herein. In some cases, addition of supplemental processed DNA (e.g., a second plurality of nucleic acid molecules) to a first plurality of nucleic acid molecules (e.g., comprising cfDNA of a biological sample) may increase a binder's selectivity for one or more (e.g., a plurality of) methylated regions of the first plurality of nucleic acid molecules. In some cases, supplemental processed DNA (e.g., the second plurality of nucleic acid molecules) may be added to the first plurality of nucleic acid molecules in an amount sufficient to bring the combined mixture of nucleic acid molecules to a desired total mass. In some cases, a desired total mass for use in a method or system described herein can be from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, or less than 20 ng. In some cases, an amount of supplemental processed DNA from 1 ng to 5 ng, from 5 ng to 10 ng, from 10 ng to 20 ng, from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, less than 20 ng, less than 10 ng, or less than 5 ng can be added to a first plurality of nucleic acid molecules (e.g., to bring the total mixture of the supplemental processed DNA and the first plurality of nucleic acid molecules to the desired total mass). In some embodiments, the present disclosure comprises methods and systems for filling in the sample with an amount of supplemental processed DNA (e.g., filler DNA) to generate a mixture sample, wherein the mixture sample comprises at least about 50 ng, 55 ng, 60 ng, 65 ng, 70 ng, 75 ng, 80 ng, 85 ng, 90 ng, 95 ng, 100 ng, 120 ng, 140 ng, 160 ng, 180 ng, 200 ng, or any amount in between the numbers of the total amount of the nucleic acid mixture. In some embodiments, the supplemental processed DNA comprises at least about 5%, 10%, 15%, 20%, 30%, 40%, 50%, 60%, 70%, 80%, 90%, or 100% methylated supplemental processed DNA with remainder being unmethylated supplemental processed DNA, and in some cases between 5% and 50%, between 10%-40%, or between 15%-30% methylated supplemental processed DNA. In some embodiments, the mixture sample comprise an amount of supplemental processed DNA from 20 ng to 100 ng, in some cases 30 ng to 100 ng, in some cases 50 ng to 100 ng. In some embodiments, the cell-free DNA from the sample and the first amount of supplemental processed DNA together comprises at least 50 ng of total DNA, in some cases at least 100 ng of total DNA.

In some cases, supplemental processed DNA may be produced by fragmentation (e.g., via sonication). In some embodiments, the supplemental processed DNA may be 50 bp to 800 bp long, in some cases 100 bp to 600 bp long, and in some cases 200 bp to 600 bp long. In some embodiments, the supplemental processed DNA is double stranded. The supplemental processed DNA may be double stranded DNA. For example, the supplemental processed DNA may be junk DNA. The supplemental processed DNA may also be endogenous or exogenous DNA. For example, the supplemental processed DNA may be non-human DNA, and in some cases, λ DNA. As used herein, “λ DNA” generally refers to Enterobacteria phage λ DNA. In some embodiments, the supplemental processed DNA has substantially no alignment to human DNA.

In some cases, the supplemental processed DNA (e.g., filler DNA) increases a fold enrichment ratio of enriching one or more methylated regions by at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least about 15 fold, at least about 20 fold, at least about 25 fold, at least about 30 fold, at least about 35 fold, at least about 40 fold, at least about 45 fold, at least about 50 fold, at least about 55 fold, at least about 60 fold, at least about 65 fold, at least about 70 fold, at least about 75 fold, at least about 80 fold, at least about 85 fold, at least about 90 fold, at least about 95 fold, at least about 100 fold, at least about 150 fold, at least about 200 fold, at least about 300 fold, at least about 400 fold, at least about 500 fold, at least about 600 fold, at least about 700 fold, at least about 800 fold, at least about 900 fold, or at least 1000 fold. In some cases, the supplemental processed DNA (e.g., filler DNA) increases a fold enrichment ratio by at most about 1 fold, at most about 2 fold, at most about 3 fold, at most about 4 fold, at most about 5 fold, at most about 6 fold, at most about 7 fold, at most about 8 fold, at most about 9 fold, at most about 10 fold, at most about 15 fold, at most about 20 fold, at most about 25 fold, at most about 30 fold, at most about 35 fold, at most about 40 fold, at most about 45 fold, at most about 50 fold, at most about 55 fold, at most about 60 fold, at most about 65 fold, at most about 70 fold, at most about 75 fold, at most about 80 fold, at most about 85 fold, at most about 90 fold, at most about 95 fold, at most about 100 fold, at most about 150 fold, at most about 200 fold, at most about 300 fold, at most about 400 fold, at most about 500 fold, at most about 600 fold, at most about 700 fold, at most about 800 fold, at most about 900 fold, or at most 1000 fold. In some cases, the supplemental processed DNA (e.g., filler DNA) increases a fold enrichment ratio by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 300 fold, about 400 fold, about 500 fold, about 600 fold, about 700 fold, about 800 fold, about 900 fold, or 1000 fold.

### Samples

A sample can be any biological sample isolated from a subject. For example, a sample may comprise, without limitation, bodily fluid, whole blood, platelets, serum, plasma, stool, white blood cells or leukocytes, endothelial cells, tissue biopsies, synovial fluid, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, fluid from nasal brushings, fluid from a pap smear, or any other bodily fluids. A bodily fluid may include saliva, blood, or serum. A sample may also be a tumor sample, which may be obtained from a subject by various approaches, including, but not limited to, venipuncture, excretion, ejaculation, massage, biopsy, needle aspirate, lavage, scraping, surgical incision, or intervention or other approaches. A sample may be a cell-free sample (e.g., substantially free of cells). DNA samples may be denatured, for example, using sufficient heat.

The sample may be taken from a subject with a disease or disorder. The sample may be taken from a subject suspected of having a disease or a disorder. The sample may be taken from a subject with more than one disease or disorder. The sample may be taken from a subject suspected of having more than one disease or disorder. In some embodiments, the sample may be obtained before and/or after treatment of a subject with a disease or disorder. Samples may be obtained from a subject during a treatment or a treatment regime. Multiple samples may be obtained from a subject to monitor the effects of the treatment over time. The disease or disorder may be a cancer. Specific examples of cancer types include suitable for detection with the methods according to the disclosure include acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, colorectal cancer, hepatobiliary cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma Merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilm's tumor. In an embodiment, the cancer is head and neck squamous cell carcinoma.

In some embodiments, the cancer can be early-stage cancer. In some embodiments, early-stage cancer can be cancer that is localized to a small area and/or has not spread to distant regions or to nearby tissues. In some embodiments, early-stage cancer can be stage I cancer. In some embodiments, the cancer can be stage I, stage II, stage III, or stage IV cancer.

In some embodiments, the cancer can be low-shedding tumors (e.g., bladder, breast, endometrial, prostate, or renal). Low-shedding tumors can shed low levels of ctDNA. In some cases, low-shedding tumors can have low ctDNA burden. In some cases, low levels of ctDNA can be found in early-stage cancer.

The sample may be taken from a healthy individual. In some cases, samples may be taken longitudinally from the same individual. In some cases, samples acquired longitudinally may be analyzed with the goal of monitoring individual health and early detection of health issues (e.g., early-stage cancer). In some embodiments, the sample may be collected at a home setting or at a point-of-care setting and subsequently transported by a mail delivery, courier delivery, or other transport method prior to analysis. For example, a home user may collect a blood spot sample through a finger prick, which blood spot sample may be dried and subsequently transported by mail delivery prior to analysis. In some cases, samples acquired longitudinally may be used to monitor response to stimuli expected to impact healthy, athletic performance, or cognitive performance. Non-limiting examples include response to medication, dieting, or an exercise regimen.

In some embodiments, the present disclosure provides a system, method, or kit that includes or uses one or more biological samples. The one or more samples used herein may comprise any substance containing or presumed to contain nucleic acids. A sample may include a biological sample obtained from a subject. In some embodiments, a biological sample is a liquid sample.

In some embodiments, the sample comprises less than about 100 ng, 90 ng, 80 ng, 75 ng, 70 ng, 60 ng, 50 ng, 40 ng, 30 ng, 20 ng, 10 ng, 5 ng, 1 ng or any amount in between the numbers of cell-free nucleic acid molecules. Further, in some embodiments, the sample comprises less than about 1 pg, less than about 5 pg, less than about 10 pg, less than about 20 pg, less than about 30 pg, less than about 40 pg, less than about 50 pg, less than about 100 pg, less than about 200 pg, less than about 500 pg, less than about 1 ng, less than about 5 ng, less than about 10 ng, less than about 20 ng, less than about 30 ng, less than about 40 ng, less than about 50 ng, less than about 100 ng, less than about 200 ng, less than about 500 ng, less than about 1000 ng, or any amount in between the numbers of cell-free nucleic acid molecules.

In some cases, creation or provision of a plurality of nucleic acid molecules from a biological sample can comprise performing one or more of end-repair, A-tailing, and adapter ligation on the plurality of nucleic acid molecules (e.g., after purification from the biological sample).

In some embodiments, a sample may be taken at a first time point and sequenced, and then another sample may be taken at a subsequent time point and sequenced. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, a medical treatment to measure the disease's progression or regression in response to the medical treatment.

After obtaining a sample from the subject, the sample may be processed to generate datasets indicative of a disease or disorder of the subject. For example, a presence, absence, or quantitative assessment of cell-free nucleic acid molecules (e.g., ctDNA molecules) of the sample at a panel of cancer-associated genomic loci or microbiome-associated loci may be indicative of a cancer of the subject. Processing the sample obtained from the subject may comprise (i) subjecting the sample to conditions that are sufficient to isolate, enrich, or extract a plurality of cell-free nucleic acid molecules, and (ii) assaying the plurality of cell-free nucleic acid molecules to generate the dataset (e.g., nucleic acid sequences). In some embodiments, a plurality of cell-free nucleic acid molecules is extracted from the sample and subjected to sequencing to generate a plurality of sequencing reads.

In some embodiments, the cell-free nucleic acid molecules may comprise cell-free ribonucleic acid (cfRNA) or cell-free deoxyribonucleic acid (cfDNA). The cell-free nucleic acid molecules (e.g., cfRNA or cfDNA) may be extracted from the sample by a variety of methods. The cell-free nucleic acid molecule may be enriched by a plurality of probes configured to enrich nucleic acid (e.g., RNA or DNA) molecules corresponding to a panel of cancer-associated genomic loci. The probes may have sequence complementarity with nucleic acid sequences from one or more of the panel of cancer-associated genomic loci. The panel of cancer-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci. The probes may be nucleic acid molecules (e.g., RNA or DNA) having sequence complementarity with nucleic acid sequences (e.g., RNA or DNA) of the one or more genomic loci (e.g., cancer-associated genomic loci). These nucleic acid molecules may be primers or enrichment sequences. The assaying of the sample using probes that are selective for the one or more genomic loci (e.g., cancer-associated genomic loci or microbiome-associated loci) may comprise use of array hybridization, polymerase chain reaction (PCR), or nucleic acid sequencing (e.g., RNA sequencing or DNA sequencing).

Certain methods of capturing cell-free methylated DNA are described in WO 2017/190215 and WO 2019/010564, both of which are incorporated by reference in their entireties and for all purposes.

### Sequencing Libraries

Various assays may be used in methods of the present disclosure, such as library preparation (which may include polymerase chain reaction (PCR)) followed by sequencing (e.g., next-generation sequencing, Sanger sequencing, etc.). Next-generation sequencing (NGS) techniques, also known as high-throughput sequencing, may include various sequencing technologies including: Illumina (Solexa) sequencing, Roche 454 sequencing, Ion torrent: Proton/PGM sequencing, SOLiD sequencing, or long reads sequencing. NGS allow for the sequencing of DNA and RNA much more quickly and cheaply than the previously used Sanger sequencing. In some embodiments, said sequencing is optimized for short read sequencing.

Sequencing libraries that are hypermethylated may improve the specificity, the sensitivity, and/or the efficiency of methods and systems for processing nucleic acids. For example, hypermethylated sequencing libraries may improve the specificity, the sensitivity, and/or the efficiency of assays for determining the presence and/or sequence identity of a nucleic acid sequence. A hypermethylated sequencing library may comprise a plurality of nucleic acids and/or fragments thereof. In some cases, a hypermethylated sequencing library may comprise a plurality of nucleic acid molecules (e.g., a population of nucleic acids and/or fragments thereof). The plurality of nucleic acid molecules may comprise all or a portion of a first plurality of nucleic acid molecules, e.g., wherein the first plurality of nucleic acid molecules comprises one or more nucleic acid molecules that comprise a methylated nucleic acid residue and one or more nucleic acid molecules that does not comprise a methylated nucleic acid residue. In some cases, a methylated nucleic acid may comprise one or more methylated nucleic acid residues. For instance, a methylated nucleic acid may comprise one or more methylated cytosines (e.g., one or more 5-methylcytosines (5mC) and/or one or more 5-hydroxymethylcytosines (5hmC)). A plurality of nucleic acid molecules (e.g., a plurality of nucleic acid molecules derived from a biological sample) may be hypermethylated and enriched by using a binder, e.g., as described herein, to form a hypermethylated sequencing library which can be used as a novel background as opposed to a whole-genome background for use in analysis of cfDNA. In some cases, DNA may be hypermethylated before use of a binder to create a sequencing library with a novel background. The novel background sequencing library may comprise a set of background genomic regions that are enriched by the binder.

In some cases, the plurality of nucleic acids may be subjected to target specific enrichment. For example, nucleic acids may be pulldown via a capture probe to enrich the sequencing library for a given sequence. In another example, nucleic acids may be amplified with primers to enrich the sequencing library for a given sequence. The capture probes or primers may be specific to a specific gene, non-coding region, or other sequence. For example, the nucleic acids may be enriched for one or more genes. The one or more genes may be genes that are associated with cancer. For example, the one or more genes may be genes that are previously determined as relevant for a cancer type. For example, the nucleic acids may be enriched for genes or sequences that have a previously identified methylation state, or previously identified number of methylated nucleotides. The target specific enrichment may be performed during the preparation of the sequencing library. For example, nucleic acids may be enriched or depleted from methylated nucleic acid (as described in this disclosure) and then the nucleic acids may be subjected to target specific enrichment.

### Nucleic Acid Molecule Sequencing

The present disclosure provides methods and technologies for determining the sequence of nucleotide bases in one or more polynucleotides. The polynucleotides may be, for example, nucleic acid molecules such as deoxyribonucleic acid (DNA) or ribonucleic acid (RNA), including variants or derivatives thereof (e.g., single stranded DNA). Sequencing may be performed by next-generation sequencing.

Further, any sequencing methods that provide fragment length such as paired-end sequencing may be utilized. Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

In some embodiments, the sequencing reads are obtained via a next-generation sequencing method or a next-next-generation sequencing method. In some embodiments, the sequencing methods comprise cfMeDIP sequencing, e.g., comprising processes or systems as described by Shen et al., (“Sensitive tumor detection and classification using plasma cell-free DNA methylomes,” (2018) Nature), which is incorporated herein in its entirety. In some embodiments, sequencing can be of a whole genome. In some embodiments, sequencing can be of a whole exome. In some embodiments, sequencing can be of a partial genome. In some embodiments, sequencing can be of a targeted genome. In some embodiments, sequencing can be of a whole methylome. In some embodiments, sequencing can be of a whole methylome without sequencing the whole genome. In some embodiments, sequencing a whole methylome can be captured without the need of predefined target panels. In some embodiments, sequencing can be of a partial methylome. In some embodiments, sequencing can be of a targeted methylome. In some embodiments, sequencing can be performed using methyl-CpG-binding domain sequencing (MBD-seq). In some cases, MBD-seq can comprise capture (e.g., via a binder, such as an antibody specific to a species of methylated nucleotide) of double-stranded, methylated DNA fragments for sequencing of methylation-enriched DNA fragment libraries. In some embodiments, the sequencing methods comprises Cancer Personalized Profiling by deep Sequencing (CAPP-Seq), which is a next-generation sequencing based method used to quantify circulating DNA in cancer (ctDNA). This method may be generalized for any cancer type that is documented to have recurrent mutations and may detect one molecule of mutant DNA in 10,000 molecules of healthy DNA. In some embodiments, the sequencing can comprise a chemical conversion. In some embodiments, the sequencing can comprise bisulfite sequencing. In some embodiments, the sequencing does not comprise bisulfite sequencing.

The sequencing may comprise targeted sequencing. For example, the sequencing reactions may comprise capture probes that are specific to regions of interest. The use of targeted sequencing may increase the amount of reads that are specific to regions that are informative (e.g., related to a DMR, or usable for distinguishing healthy subject vs a subject suffering from a disease). The capture probes may comprise one or more probes that are complementary or homologous to regions that comprises have one or more sites that are amenable to enzymatic methylation. The capture probes may comprise one or more probes that are complementary or homologous to regions that comprises have one or more sites that have substantially no methylation in a healthy or non-disease control. The capture probes may comprise one or more probes that are complementary or homologous to regions that have a known methylation state. The targeted sequencing may target one or more regions that are known to be present in multiple types of cancers.

In some embodiments, the sequencing preserves fragment length and/or end motif. Non-limiting examples of end motif include 5′ end motifs, 3′ end motifs, 6-mer end motifs, 5-mer end motifs, 4-mer end motifs (e.g., CCCA, CCTG, CCAG, CCAA, CCAT, CCTG, CCAA, CCCT, CCTC, TGTG, TGTT, CCTA, TATT, CCAC, TCTT, CCCC, TATA, TAAA, AAAA, TTTT, or other variations thereof). Preservation of fragment length and end motif can enable multi-omic assessments with a single assay, which can increase efficiency and increase performance where ctDNA is low. In some embodiments, the sequencing can comprise an enzymatic conversion. In some cases, an enzymatic conversion can comprise of TET2 and an oxidation enhancer. In some cases, an enzymatic conversion can further comprise APOBEC. In some embodiments, the sequencing does not comprise an enzymatic conversion. In some embodiments, lack of conversion (e.g., chemical or enzymatic) can preserve the quality of the DNA and the four DNA bases (e.g., adenine, cytosine, guanine, thymine). Preserving the quality of the DNA can allow more reads to pass quality control filters that uniquely align to the human genome.

In some cases, a sample or portion thereof (e.g., a plurality of nucleic acids of a sample) may be subjected to library preparation before sequencing. In short, after end-repair and A-tailing, the samples are ligated to nucleic acid adapters and digested using enzymes.

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (e.g., a UMI). In some embodiments, a barcode is non-unique, and barcode sequences may be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule). A barcode, UMI, or tag may be a known sequence used to associate a polynucleotide or fragment thereof with an input or target nucleic acid molecule or fragment thereof. A barcode, UMI, or tag may comprise natural nucleotides or non-natural (e.g., modified) nucleotides (e.g., as described herein). A barcode sequence may be contained within an adapter sequence such that the barcode sequence may be contained within a sequencing read. A barcode sequence may comprise at least 4, 5, 6, 7, 8, 9, 10, 11, 12, 13, 14, 15, 16, or more nucleotides in length. In some cases, a barcode sequence may be of sufficient length and may be sufficiently different from another barcode sequence to allow the identification of a sample based on a barcode sequence with which it is associated. A barcode sequence, or a combination of barcode sequences, may be used to tag and subsequently identify an “original” nucleic acid molecule or fragment thereof (e.g., a nucleic acid molecule or fragment thereof present in a sample from a subject). In some cases, a barcode sequence, or a combination of barcode sequences, is used in conjunction with endogenous sequence information to identify an original nucleic acid molecule or fragment thereof. For example, a barcode sequence, or a combination of barcode sequences, may be used with endogenous sequences adjacent to a barcode, UMI, or tag (e.g., the beginning and end of the endogenous sequences).

As described herein, the prepared libraries may be combined with filler nucleic acids (e.g., filler λ DNAs) to minimize the effect of low abundance ctDNA in the prepared libraries and generate mixed samples. In some embodiments, when the disease/condition is a locoregional (non-metastatic) cancer, the amount of ctDNA can be low and may not be easily and accurately measured and quantified. In such cases, the mixed samples may be brought to at least about 50 ng, 80 ng, 100 ng, 120 ng, 150 ng, or 200 ng and are subjected to further enrichment.

Processing a nucleic acid molecule or fragment thereof may comprise performing nucleic acid amplification. Amplification prior to sequencing of nucleic acids (e.g., enriched methylated nucleic acids) may generate more sequence reads. Amplification may allow for more stable nucleic acids to be generated (e.g., double stranded nucleic acid compared to partially single stranded or single stranded nucleic acids), which may in turn allow for of the nucleic acid to be stored for longer periods of time without degradation. For example, any type of nucleic acid amplification reaction may be used to amplify a target nucleic acid molecule or fragment thereof and generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Examples of PCR include, but are not limited to, quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification may be isothermal or may comprise thermal cycling. and/or with the length of the endogenous sequence. In some cases, PCR amplification comprises at least 10 cycles, at least 11 cycles, at least 12 cycles, at least 13 cycles, at least 14 cycles of amplification.

Amplification to generate nucleic acids suitable for sequencing may be performed on nucleic acids that are bound to or attached to solid substrate (e.g., beads). For example, as described elsewhere in this disclosure, methylation binding molecules may be attached to solid substrates, and the methylation binding molecules may bind nucleic acids. The bound nucleic acids may be amplified and may generate amplicons that are not bound to a binding molecule or solid substrate. Amplification of nucleic acids that are bound to a solid substrate may allow for improved throughput, for example, by reducing the need for washes or buffer exchange that may occur upon elution or removal of nucleic acids from a solid substrate.

Processing a nucleic acid molecule or fragment thereof may comprise the addition of bead that may bind to nucleic acids. The binding of nucleic acids may allow for nucleic acids to a be specifically bound and allow for enzymes or contaminants to be removed from the nucleic acid sample. The beads (e.g., SPRI beads) may bind to nucleic acids of the libraries and may be washed or separated to remove contaminants. The nucleic acids may be eluted from the beads and then may be collected. This may be performed by removing the beads from a nucleic acid sample. For example, the beads may be magnetic beads and may be subjected to a magnetic field to remove the beads. The beads may be subjected to multiple iterations of removal process to reduce or minimize carryover of beads into later processing reactions.

### Binders

A binder may be used to deplete or enrich for a population of nucleic acid molecules (e.g., a plurality of nucleic acid molecules derived from a biological sample). In some cases, a binder can be used to deplete or enrich for a plurality of nucleic acid molecules of one or more nucleic acid molecules having a methylation level at or above a threshold methylation level (e.g., by binding to one or more methylated nucleotides of the one or more nucleic acid molecules). A binder may be used to enrich a population of nucleic acid molecules (e.g., a plurality of nucleic acids derived from a biological sample). The binder may be a molecule that binds specifically to methylated nucleic acids or methylated nucleotides. In some cases, a binder can be specific to one or more methylated nucleotide species (e.g., 5-methylcytosine (5mC), 5-hydroxymethylcytosine (5hmC), 4-methylcytosine (4mC), or 6-methyladenine (6 mA)). In some cases, a binder can be selected from the group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof. In some cases, the binder can be an anti-5-methylcytosine antibody or a derivative thereof. In some embodiments, the binder is a protein comprising a Methyl-CpG-binding domain. One such protein is MBD2 protein. As used herein, “Methyl-CpG-binding domain (MBD)” generally refers to certain domains of proteins and enzymes that are approximately 70 residues long and bind to DNA that contains one or more symmetrically methylated CpGs. The MBD of MeCP2, MBD1, MBD2, MBD4 and BAZ2 mediates binding to DNA, and in cases of MeCP2, MBD1 and MBD2, preferentially to methylated CpG. Human proteins MECP2, MBD1, MBD2, MBD3, and MBD4 comprise a family of nuclear proteins related by the presence in each of a methyl-CpG-binding domain (MBD). Each of these proteins, with the exception of MBD3, is capable of binding specifically to methylated DNA. The binder may comprise a biotin, and may allow the binder to couple to, or bind to, a streptavidin.

In other embodiments, the binder is an antibody and capturing cell-free methylated DNA comprises immunoprecipitating the cell-free methylated DNA using the antibody. As used herein, “immunoprecipitation” generally refers a technique of precipitating an antigen (such as polypeptides and nucleotides) out of solution using an antibody that specifically binds to that particular antigen. This process may be used to isolate and concentrate a particular protein or DNA from a sample and may require that the antibody be coupled to a solid substrate at some point in the procedure. The solid substrate includes for example beads, such as magnetic beads. Other types of beads and solid substrates may be used. Various proteins or chemical moieties may be present on the solid substrate that may allow for coupling or binding to a binder. For example, the solid substrate may be able to bind or couple to a molecule that binds specifically to methylated nucleic acids. For example, the solid substrate may comprise streptavidin and may be able to bind to a biotinylated antibody. In another example, the solid substrate may comprise a protein A and may be able to bind to a Fc domain of an antibody.

In various aspects, binders (e.g., a methylation binding molecule) are added to a sample to bind to nucleic acids. The binders may be coupled to a solid substrate (e.g., a magnetic bead) and this complex (e.g., a methylated nucleic acid capture reagent) may be added to sample. The complex may initially be generated via a prior incubation without the sample present. For example, an anti-5-mC antibody may be incubated with protein A bead and allow for the antibody to bind to, or couple to, the protein A bead via a Fc domain. This may allow the bead to be saturated with the antibody, and this complex may be able to bind to methylated nucleic acid. Upon generating the complex, the complex may be added to the sample to bind to nucleic acids. The binder and the solid substrate may also be added at a same time (or substantially the same time) to a sample or may be added sequentially. For example, a magnetic protein A bead and antibody may be added at the same time to a sample and allow binding of the antibody to a nucleic acids and allow binding of the antibody to magnetic protein A bead. In another example, an antibody may be initially added to a sample to allow binding of the nucleic acids to the antibody, followed by addition of a bead to bind to the antibody which bound to the nucleic acids. The generation of the complex via prior incubation, simultaneous addition, or sequential addition, each may comprise advantages. For example, prior incubation may allow for a stable complex of the binder and solid substrate to be formed without potential steric interference of nucleic acids.

For example, a 5-mC antibody (e.g., wherein the 5-mC antibody specifically binds to 5-methylcytosine) may be used as a binder. For the immunoprecipitation procedure, in some embodiments at least 0.05 μg of the antibody is added to the sample, while in some embodiments at least 0.16 μg of the antibody is added to the sample. In some cases, 0.05 μg to 0.80 μg, 0.16 μg to 0.80 μg, 0.40 μg to 0.80 μg, 0.16 μg to 0.40 μg, 0.10 μg to 0.80 μg, 0.20 μg to 0.60 μg, 0.30 μg to 0.50 μg, or 0.40 μg to 0.50 μg of the antibody can be used. To confirm the immunoprecipitation reaction, in some embodiments the method described herein further comprises the operation of adding a second amount of control DNA to the sample.

In some embodiments, the immunoprecipitation process is optimized, wherein optimization of the immunoprecipitation can comprise changing the binder (e.g., antibody) used, adjusting the concentration of binder, and/or adjusting the length of time that the binder is allowed to capture cell-free methylated DNA.

### Methods of Processing Methylated Nucleic Acids

The present disclosure provides methods and systems of processing a cell-free nucleic acid sample from a subject for detecting a methylation event.

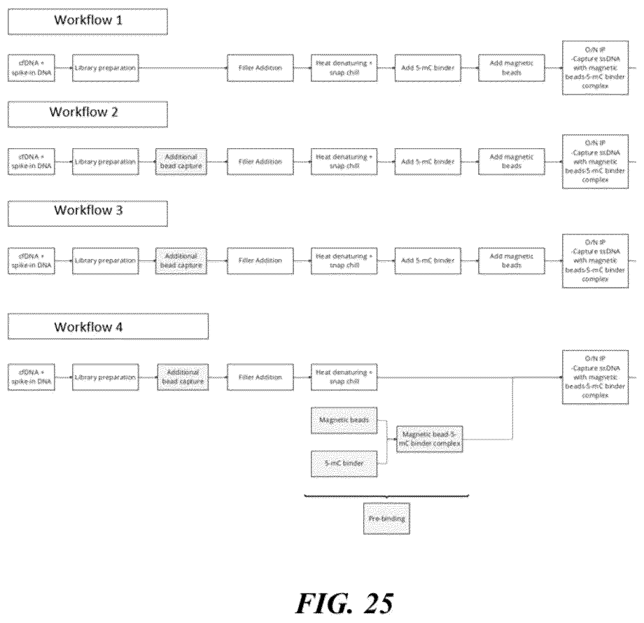

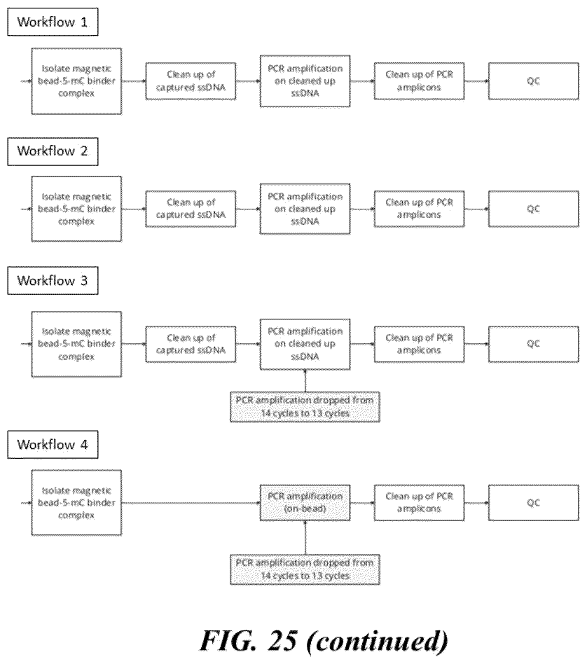

In some embodiments, the methods, as illustrated in FIG. 25 (Workflow 1), comprise: (a) providing a plurality of nucleic acid molecules (e.g., cfDNA, cfDNA with spike-in DNA) derived from a nucleic acid sample; (b) undergoing a library preparation (e.g., end-repair, A-tail, adaptor ligation) with one or more custom adaptors to generate a library; (c) adding a plurality of filler nucleic acid molecules to generate a sample mixture; (d) heat denaturing and snap chilling the sample mixture; (e) subjecting the sample mixture to immunoprecipitation to yield an enriched sample comprising a plurality of methylated nucleic acid molecules; and (f) preparing the enriched sample for sequencing. In some cases of the method, (e) subjecting the sample mixture to immunoprecipitation further comprises: i) adding a binder as disclosed herein to the sample mixture; ii) adding a solid substrate (e.g., magnetic solid substrate) to the sample mixture; iii) incubating the binder and the solid substrate with the sample mixture for a sufficient amount of time (e.g., overnight) to capture the enriched sample comprising the plurality of methylated nucleic acid molecules; and iii) isolating the binder and the solid substrate from the enriched sample. In some cases of the method, (f) preparing the enriched sample for sequencing further comprises: i) cleaning up the enriched sample to generate a cleaned up enriched sample; ii) performing PCR amplification on the cleaned up enriched sample to yield a plurality of PCR amplicons; and iii) cleaning up the PCR amplicons before sequencing. In some cases, the PCR amplification can be for 14 cycles of amplification.

In some embodiments, the methods, as illustrated in FIG. 25 (Workflow 2), comprise: (a) providing a plurality of nucleic acid molecules (e.g., cfDNA, cfDNA with spike-in DNA) derived from a nucleic acid sample; (b) undergoing a library preparation (e.g., end-repair, A-tail, adaptor ligation) with one or more custom adaptors to generate a library; (c) performing a post-library preparation clean-up with a solid substrate (e.g., magnetic solid substrate); (d) adding a plurality of filler nucleic acid molecules to generate a sample mixture; (e) heat denaturing and snap chilling the sample mixture; (f) subjecting the sample mixture to immunoprecipitation to yield an enriched sample comprising a plurality of methylated nucleic acid molecules; and (g) preparing the enriched sample for sequencing. In some cases of the methods (c) performing a post-library preparation clean-up comprises an additional capture of the solid substrate (e.g., magnetic solid substrate) by placing the prepped library against a device (e.g., magnetic rack) to capture any remaining solid substrates. In some cases of the methods (f) subjecting the sample mixture to immunoprecipitation further comprises: i) adding a binder as disclosed herein to the sample mixture; ii) adding a solid substrate (e.g., magnetic solid substrate) to the sample mixture; iii) incubating the binder and the solid substrate with the sample mixture for a sufficient amount of time (e.g., overnight) to capture the enriched sample comprising the plurality of methylated nucleic acid molecules; and iii) isolating the binder and the solid substrate from the enriched sample. In some cases of the methods, (g) preparing the enriched sample for sequencing further comprises: i) cleaning up the enriched sample to generate a cleaned up enriched sample; ii) performing PCR amplification on the cleaned up enriched sample to yield a plurality of PCR amplicons; and iii) cleaning up the PCR amplicons before sequencing. In some cases, the PCR amplification can be for 14 cycles of amplification.

In some embodiments, the methods as illustrated in FIG. 25 (Workflow 3), comprise: (a) providing a plurality of nucleic acid molecules (e.g., cfDNA, cfDNA with spike-in DNA) derived from a nucleic acid sample; (b) undergoing a library preparation (e.g., end-repair, A-tail, adaptor ligation) with one or more custom adaptors to generate a library; (c) performing a post-library preparation clean-up with a solid substrate (e.g., magnetic solid substrate); (d) adding a plurality of filler nucleic acid molecules to generate a sample mixture; (e) heat denaturing and snap chilling the sample mixture; (f) subjecting the sample mixture to immunoprecipitation to yield an enriched sample comprising a plurality of methylated nucleic acid molecules; and (g) preparing the enriched sample for sequencing. In some cases of the method, (c) performing a post-library preparation clean-up comprises an additional capture of the solid substrate (e.g., magnetic solid substrate) by placing the prepped library against a device (e.g., magnetic rack) to capture any remaining solid substrates. In some cases of the methods, (f) subjecting the sample mixture to immunoprecipitation further comprises: i) adding a binder as disclosed herein to the sample mixture; ii) adding a solid substrate (e.g., magnetic solid substrate) to the sample mixture; iii) incubating the binder and the solid substrate with the sample mixture for a sufficient amount of time (e.g., overnight) to capture the enriched sample comprising the plurality of methylated nucleic acid molecules; and iii) isolating the binder and the solid substrate from the enriched sample. In some cases of the methods, (g) preparing the enriched sample for sequencing further comprises: i) cleaning up the enriched sample to generate a cleaned up enriched sample; ii) performing PCR amplification on the cleaned up enriched sample to yield a plurality of PCR amplicons; and iii) cleaning up the PCR amplicons before sequencing. In some cases, the PCR amplification can be for 13 cycles of amplification.

In some embodiments, the methods, as illustrated in FIG. 25 (Workflow 4), comprise (a) providing a plurality of nucleic acid molecules (e.g., cfDNA, cfDNA with spike-in DNA) derived from a nucleic acid sample; (b) undergoing a library preparation (e.g., end-repair, A-tail, adaptor ligation) with one or more custom adaptors to generate a library; (c) performing a post-library preparation clean-up with a solid substrate (e.g., magnetic solid substrate); (d) adding a plurality of filler nucleic acid molecules to generate a sample mixture; (e) heat denaturing and snap chilling the sample mixture; (f) subjecting the sample mixture to immunoprecipitation to yield an enriched sample comprising a plurality of methylated nucleic acid molecules; and (g) preparing the enriched sample for sequencing. In some cases of the methods, (c) performing a post-library preparation clean-up comprises an additional capture of the solid substrate (e.g., magnetic solid substrate) by placing the prepped library against a device (e.g., magnetic rack) to capture any remaining solid substrates. In some cases of the methods (f) subjecting the sample mixture to immunoprecipitation further comprises i) incubating a binder as disclosed herein with a solid substrate (e.g., magnetic solid substrate) to generate a methylated nucleic acid capture reagent; ii) adding the methylated nucleic acid capture reagent to the sample mixture; and ii) isolating the methylated nucleic acid capture reagent after incubation of the methylated nucleic acid capture reagent and the sample mixture for a sufficient amount of time (e.g., overnight) to yield the enriched sample comprising the plurality of methylated nucleic acid molecules. In some cases of the methods, (g) preparing the enriched sample for sequencing further comprises: i) cleaning up the enriched sample to generate a cleaned up enriched sample; ii) performing PCR amplification on the cleaned up enriched sample to yield a plurality of PCR amplicons; and iii) cleaning up the PCR amplicons before sequencing. In some cases, the PCR amplification can be for 13 cycles of amplification.

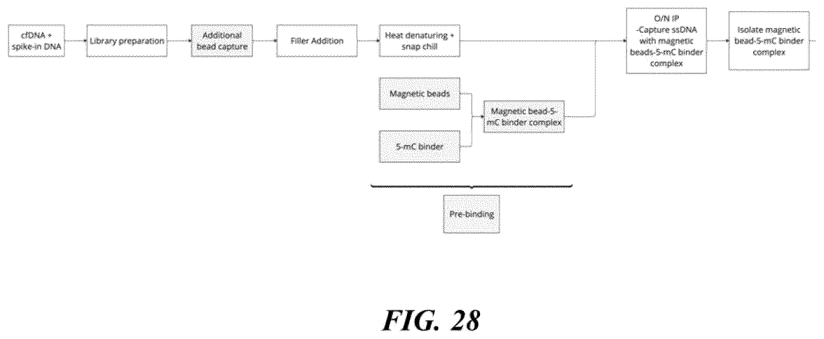

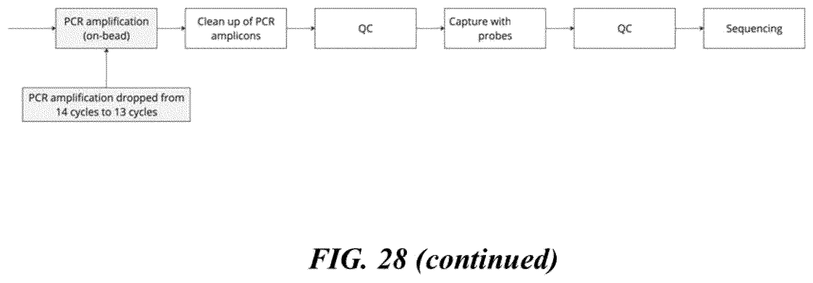

In some embodiments, the methods, as illustrated in FIG. 28, comprise (a) providing a plurality of nucleic acid molecules (e.g., cfDNA, cfDNA with spike-in DNA) derived from a nucleic acid sample; (b) undergoing a library preparation (e.g., end-repair, A-tail, adaptor ligation) with one or more custom adaptors to generate a library; (c) performing a post-library preparation clean-up with a solid substrate (e.g., magnetic solid substrate); (d) adding a plurality of filler nucleic acid molecules to generate a sample mixture; (e) heat denaturing and snap chilling the sample mixture; (f) subjecting the sample mixture to immunoprecipitation to yield an enriched sample comprising a plurality of methylated nucleic acid molecules; and (g) preparing the enriched sample for sequencing. In some cases of the methods, (c) performing a post-library preparation clean-up comprises an additional capture of the solid substrate (e.g., magnetic solid substrate) by placing the prepped library against a device (e.g., magnetic rack) to capture any remaining solid substrates. In some cases of the methods, (f) subjecting the sample mixture to immunoprecipitation further comprises i) incubating a binder as disclosed herein with a solid substrate (e.g., magnetic solid substrate) to generate a methylated nucleic acid capture reagent; ii) adding the methylated nucleic acid capture reagent to the sample mixture; and ii) isolating the methylated nucleic acid capture reagent after incubation of the methylated nucleic acid capture reagent and the sample mixture for a sufficient amount of time (e.g., overnight) to yield the enriched sample comprising the plurality of methylated nucleic acid molecules. In some cases of the methods, (g) preparing the enriched sample for sequencing further comprises: i) cleaning up the enriched sample to generate a cleaned up enriched sample; ii) performing PCR amplification on the cleaned up enriched sample to yield a plurality of PCR amplicons; iii) cleaning up the PCR amplicons; and iv) contacting the plurality of methylated nucleic acids with one or more nucleic acid capture probes to enrich for one or more target sequences before sequencing. In some cases, the PCR amplification can be for 13 cycles of amplification. In some cases, the one or more target sequence comprises one or more genes.

In one aspect, disclosed herein is a method or a system of processing a nucleic acid sample from a subject comprising: (a) generating a nucleic acid sample mixture comprising a plurality of methylated nucleic acids from the subject and an amount of supplemental processed DNA (e.g., filler DNA), and (b) incubating the nucleic acid sample mixture with (i) a methylation binding molecule and (ii) a solid substrate, and (c) capturing said methylated nucleic acid to enrich the nucleic acid sample mixture for the plurality of methylated nucleic acids (e.g., methylated single-stranded DNA). In some cases, an amount of supplemental processed DNA (e.g., filler DNA) is not required in (a). In some cases, an amount of supplemental processed DNA (e.g., filler DNA) comprises at least one methylated DNA molecule. In some cases, methylation binding molecule is an antibody (e.g., an anti-5-methylcytosine (anti-5mC) antibody, methyl-CpG-binding domain (MBD) protein). In some cases, the methylation binding molecule comprises biotin. In some cases, the methylation binding molecule binds to a methylated cytosine. In some cases, the solid substrate is a bead. In some cases, the solid substrate is a magnetic solid substrate. In some cases, the solid substrate comprises protein A. In some cases, the solid substrate comprises streptavidin. In some cases, the method further comprises subsequent to (a) and prior to (b), denaturing nucleic acids in said nucleic acid sample mixture. In some cases, the method further comprises, prior to (a), obtaining the nucleic acid samples from the sample and performing one or more library preparation reactions on the nucleic acids. In some cases, exogenous DNA (e.g., spike-in DNA) is mixed with the nucleic acid samples before performing one or more library preparation reactions. In some cases, subsequent to performing one or more library preparation reactions, prior to (a), the prepped library is incubated with a plurality of DNA capture beads (e.g., SPRI beads), then removed. In some cases, the method further comprises, subsequent to capturing, amplifying said methylated nucleic acid to generate amplicons of the plurality of methylated nucleic acids. In some cases, the captured methylated nucleic acids are subjected to a buffer exchange or wash reaction prior to amplifying. In some cases, the captured methylated nucleic acids are subjected to an elution reaction prior to amplifying. In some cases, the captured methylated nucleic acids are not subjected to an elution reaction prior to amplifying. In some cases, amplifying is via PCR amplification. In some cases, PCR amplification comprises at least 10 cycles, at least 11 cycles, at least 12 cycles, at least 13 cycles, at least 14 cycles of amplification. In some cases, PCR amplification is 14 cycles of amplification. In some cases, PCR amplification is 13 cycles of amplification. In some cases, the generated amplicons of methylated nucleic acid are subjected to a sequencing reaction. In some cases, the amplicons undergo clean up before sequencing. In some cases, the amplicons do not undergo clean up before sequencing. In some cases, the sequencing reaction is a sequencing by synthesis reaction. In some cases, the sequencing reaction does not comprise bisulfite sequencing. In some cases, the method or the system further comprises contacting the plurality of methylated nucleic acids with one or more nucleic acid capture probes to enrich for one or more target sequences. In some cases, the one or more target sequences comprises one or more genes.

In one aspect, disclosed herein is a method or a system of processing a nucleic acid sample from a subject comprising: (a) generating a nucleic acid sample mixture comprising a plurality of methylated nucleic acids from the subject and an amount of supplemental processed DNA (e.g., filler DNA), and (b) incubating (i) a methylation binding molecule with (ii) a solid substrate to form a methylated nucleic acid capture reagent; and (c) capturing said methylated nucleic acid by adding said methylated nucleic acid capture reagent to said nucleic acid sample mixture to enrich the nucleic acid mixture for the plurality of methylated nucleic acids. In some cases, an amount of supplemental processed DNA (e.g., filler DNA) is not required in (a). In some cases, an amount of supplemental processed DNA (e.g., filler DNA) comprises at least one methylated DNA molecule. In some cases, methylation binding molecule is an antibody (e.g., an anti-5-methylcytosine (anti-5mC) antibody, methyl-CpG-binding domain (MBD) protein). In some cases, the methylation binding molecule comprises biotin. In some cases, the methylation binding molecule binds to a methylated cytosine. In some cases, the solid substrate is a bead. In some cases, the solid substrate is a magnetic solid substrate. In some cases, the solid substrate comprises protein A. In some cases, the solid substrate comprises streptavidin. In some cases, the method further comprises prior to (b), denaturing nucleic acids in said nucleic acid sample mixture. In some cases, the method further comprises, prior to (a), obtaining the nucleic acid samples from the sample and performing one or more library preparation reactions on the nucleic acids. In some cases, exogenous DNA (e.g., spike-in DNA) is mixed with the nucleic acid samples before performing one or more library preparation reactions. In some cases, subsequent to performing one or more library preparation reactions, prior to (a), the prepped library is incubated with a plurality of magnetic beads that interact with nucleic acids (e.g., SPRI beads), and eluted from magnetic beads that interact with nucleic acid. In some cases, the sample is subjected to magnetic capture to remove the magnetic beads that interact with nucleic acids. In some cases, the sample is subjected to an additional magnetic capture to remove residual magnetic beads that interact with nucleic acids. In some cases, the method further comprises, subsequent to capturing, amplifying said methylated nucleic acid to generate amplicons of the plurality of methylated nucleic acids. In some cases, the amplifying is performed while the methylated nucleic acid is bound to the methylated nucleic acid capture reagent. In some cases, the captured methylated nucleic acids are subjected to a buffer exchange or wash reaction prior to amplifying. In some cases, the captured methylated nucleic acids are subjected to an elution reaction prior to amplifying. In some cases, the captured methylated nucleic acids are not subjected to an elution reaction prior to amplifying (e.g., when amplifying is performed while the methylated nucleic acid is bound to the methylated nucleic acid capture reagent). In some cases, amplifying is via PCR amplification. In some cases, PCR amplification comprises at least 10 cycles, at least 11 cycles, at least 12 cycles, at least 13 cycles, at least 14 cycles of amplification. In some cases, PCR amplification is 14 cycles of amplification. In some cases, PCR amplification is 13 cycles of amplification. In some cases, the generated amplicons of methylated nucleic acid are subjected to a sequencing reaction. In some cases, the amplicons undergo clean up before sequencing. In some cases, the amplicons do not undergo clean up before sequencing. In some cases, the sequencing reaction is a sequencing by synthesis reaction. In some cases, the sequencing reaction does not comprise bisulfite sequencing. In some cases, the method or the system further comprises contacting the plurality of methylated nucleic acids with one or more nucleic acid capture probes to enrich for one or more target sequences. In some cases, the one or more target sequences comprises one or more genes.

In one aspect, disclosed herein is a method or a system of processing a nucleic acid sample from a subject comprising: (a) providing a nucleic acid sample comprising a plurality of methylated nucleic acids; (b) incubating (i) a methylated binding molecule with (ii) a solid substrate to form a methylated nucleic acid capture reagent; (c) capturing said methylated nucleic acid by adding said methylated nucleic acid capture reagent to said nucleic acid sample, thereby generating solid substrate bound methylated nucleic acid; (d) amplifying said solid substrate bound methylated nucleic acid to generate amplicons of said methylated nucleic acid. In some cases, the amplifying is performed while the methylated nucleic acid is bound to the methylated nucleic acid capture reagent. In some cases, the capture methylated nucleic acid are not subjected to an elution reaction prior to amplifying. In some cases, methylation binding molecule is an antibody (e.g., an anti-5-methylcytosine (anti-5mC) antibody, methyl-CpG-binding domain (MBD) protein). In some cases, the methylation binding molecule comprises biotin. In some cases, the methylation binding molecule binds to a methylated cytosine. In some cases, the solid substrate is a bead. In some cases, the solid substrate is a magnetic solid substrate. In some cases, the solid substrate comprises protein A. In some cases, the solid substrate comprises streptavidin. In some cases, the method further comprises subsequent to (a) and prior to (b), denaturing nucleic acids in said nucleic acid sample mixture. In some cases, the method further comprises, prior to (a), obtaining the nucleic acid samples from the sample and performing one or more library preparation reactions on the nucleic acids. In some cases, exogenous DNA (e.g., spike-in DNA) is mixed with the nucleic acid samples before performing one or more library preparation reactions. In some cases, subsequent to performing one or more library preparation reactions, prior to (a), the prepped library is incubated with a plurality of magnetic beads that interact with nucleic acids (e.g., SPRI beads), and eluted from magnetic beads that interact with nucleic acid. In some cases, the sample is subjected to magnetic capture to remove the magnetic beads that interact with nucleic acids. In some cases, the sample is subjected to an additional magnetic capture to remove residual magnetic beads that interact with nucleic acids. In some cases, the captured methylated nucleic acids are not subjected to an elution reaction prior to amplifying. In some cases, amplifying is via PCR amplification. In some cases, PCR amplification comprises at least 10 cycles, at least 11 cycles, at least 12 cycles, at least 13 cycles, at least 14 cycles of amplification. In some cases, PCR amplification is 14 cycles of amplification. In some cases, PCR amplification is 13 cycles of amplification. In some cases, the generated amplicons of methylated nucleic acid are subjected to a sequencing reaction. In some cases, the amplicons undergo clean up before sequencing. In some cases, the amplicons do not undergo clean up before sequencing. In some cases, the sequencing reaction is a sequencing by synthesis reaction. In some cases, the sequencing reaction does not comprise bisulfite sequencing. In some cases, the method or the system further comprises contacting the plurality of methylated nucleic acids with one or more nucleic acid capture probes to enrich for one or more target sequences. In some cases, the one or more target sequences comprises one or more genes.

In one aspect, disclosed herein is a method or a system of processing a nucleic acid sample from a subject comprising: (a) generating a nucleic acids sample mixture comprising a plurality of methylated nucleic acids from said subject and an amount of supplemental processed DNA (e.g., filler DNA), wherein said filler DNA comprises at least one methylated DNA molecule; (b) capturing said methylated nucleic acid by adding a capture reagent that comprises a solid substrate to said nucleic acid sample mixture, thereby generating solid substrate bound methylated nucleic acid; and (c) amplifying the solid substrate bound methylated nucleic acid to generate amplicons of methylated nucleic acid. In some cases, an amount of supplemental processed DNA (e.g., filler DNA) comprises at least one methylated DNA molecule. In some cases, the amplifying is performed while the methylated nucleic acid is bound to the methylated nucleic acid capture reagent. In some cases, the capture methylated nucleic acid are not subjected to an elution reaction prior to amplifying. In some cases, methylation binding molecule is an antibody (e.g., an anti-5-methylcytosine (anti-5mC) antibody, methyl-CpG-binding domain (MBD) protein). In some cases, the methylation binding molecule comprises biotin. In some cases, the methylation binding molecule binds to a methylated cytosine. In some cases, the solid substrate is a bead. In some cases, the solid substrate is a magnetic solid substrate. In some cases, the solid substrate comprises protein A. In some cases, the solid substrate comprises streptavidin. In some cases, the method further comprises subsequent to (a) and prior to (b), denaturing nucleic acids in said nucleic acid sample mixture. In some cases, the method further comprises, prior to (a), obtaining the nucleic acid samples from the sample and performing one or more library preparation reactions on the nucleic acids. In some cases, exogenous DNA (e.g., spike-in DNA) is mixed with the nucleic acid samples before performing one or more library preparation reactions. In some cases, subsequent to performing one or more library preparation reactions, prior to (a), the prepped library is incubated with a plurality of magnetic beads that interact with nucleic acids (e.g., SPRI beads), and eluted from magnetic beads that interact with nucleic acid. In some cases, the sample is subjected to magnetic capture to remove the magnetic beads that interact with nucleic acids. In some cases, the sample is subjected to an additional magnetic capture to remove residual magnetic beads that interact with nucleic acids. In some cases, the captured methylated nucleic acids are not subjected to an elution reaction prior to amplifying. In some cases, amplifying is via PCR amplification. In some cases, PCR amplification comprises at least 10 cycles, at least 11 cycles, at least 12 cycles, at least 13 cycles, at least 14 cycles of amplification. In some cases, PCR amplification is 14 cycles of amplification. In some cases, PCR amplification is 13 cycles of amplification. In some cases, the generated amplicons of methylated nucleic acid are subjected to a sequencing reaction. In some cases, the amplicons undergo clean up before sequencing. In some cases, the amplicons do not undergo clean up before sequencing. In some cases, the sequencing reaction is a sequencing by synthesis reaction. In some cases, the sequencing reaction does not comprise bisulfite sequencing. In some cases, the method or the system further comprises contacting the plurality of methylated nucleic acids with one or more nucleic acid capture probes to enrich for one or more target sequences. In some cases, the one or more target sequences comprises one or more genes.

In one aspect, disclosed herein is a method or a system comprising: (a) obtaining a first nucleic acid molecules from a cell free sample of a subject, (b) generating a second set of nucleic acid molecules from the first set of nucleic acid molecules or a derivative thereof, wherein said second set of nucleic acid molecules is enriched for methylation level relative to said first set of nucleic acid molecules, (c) enriching said second set of nucleic acid molecules or a derivative thereof for one or more targets to yield a third set of nucleic acid molecules; and (d) sequencing said third set of nucleic acid molecules or a derivative thereof. In some cases, the enriching comprises contacting the second set of nucleic acid molecules or derivatives thereof with one or more nucleic acid capture probes. In some cases, the generating comprises contacting the first set of nucleic acids molecules or the derivative thereof with a methylated nucleic acid capture reagent. In some cases, the methylated nucleic acid capture reagent is formed by incubating a methylation binding molecule with (ii) a solid substrate. In some cases, the solid substrate is a bead. In some cases, the solid substrate is a magnetic solid substrate. In some cases, the solid substrate comprises protein A. In some cases, the solid substrate comprises streptavidin. In some cases, the methylation binding molecule is an antibody (e.g., an anti-5-methylcytosine (anti-5mC) antibody, methyl-CpG-binding domain (MBD) protein). In some cases, the methylation binding molecule comprises biotin. In some cases, the methylation binding molecule binds to a methylated cytosine. In some cases, the method or system further comprises amplifying the second set of molecules. In some cases, the amplifying is performed while a subset of said first set of nucleic acids are bound to the methylated nucleic acid capture reagent. In some cases, amplifying is via PCR amplification. In some cases, PCR amplification comprises at least 10 cycles, at least 11 cycles, at least 12 cycles, at least 13 cycles, at least 14 cycles of amplification. In some cases, PCR amplification is 14 cycles of amplification. In some cases, PCR amplification is 13 cycles of amplification. In some cases, amplicons generated from the amplification can undergo clean up. In some cases, the sequencing is a sequencing by synthesis reaction. In some cases, the sequencing does not comprise bisulfite sequencing. In some cases, prior to the sequencing, performing one or more library preparation reactions on the third set of nucleic acid molecules. In some cases, the method or system further comprises, subsequent to the performing the one or more library preparation reactions, and prior to the sequencing, incubating the third set of nucleic acid molecules with a plurality of magnetic beads that interact with nucleic acids with a plurality of magnetic beads that interact with nucleic acids. In some cases, the method or system further comprises, subsequent to incubating the nucleic acid sample with a plurality of magnetic beads that interact with nucleic acids, subjecting the sample to a magnetic capture to remove the plurality of magnetic beads that interact with nucleic acids. In some cases, the method or system further comprises performing an additional magnetic capture. In some cases, the method or system further comprises prior to (b), adding an amount of filler DNA to the first nucleic acid molecules.

In any of the methods or systems of processing nucleic acids described herein, quality control analysis can be performed on the captured methylated nucleic acid. For example, quality control analysis can comprise measuring methylation binding specificity. In some cases, the methylation binding specificity is measured by calculating the recovery of exogenous methylated fragments. In some cases, the methylation binding specificity is measured by calculating the event of methylated fragments detected. In some cases, the methods and systems of processing nucleic acid sample from a subject described herein result in enrichment of methylated single-stranded DNA with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, or at least about 99.9% methylation specificity. In some cases, the methods and systems of processing nucleic acid sample from a subject described herein result enrichment of methylated nucleic acids by at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least about 15 fold, at least about 20 fold, at least about 25 fold, at least about 30 fold, at least about 35 fold, at least about 40 fold, at least about 45 fold, at least about 50 fold, at least about 55 fold, at least about 60 fold, at least about 65 fold, at least about 70 fold, at least about 75 fold, at least about 80 fold, at least about 85 fold, at least about 90 fold, at least about 95 fold, at least about 100 fold, at least about 150 fold, or at least about 200 fold.

### Methylation Profile

The present disclosure provides methods and systems for producing a methylation profile of a subject that has a disease/condition or is suspected of having such disease/condition, wherein the methylation profile may be used to determine whether the subject has the disease/condition or is at risk of having the disease/condition. In some cases, the methylation profile may be used to determine whether the subject has or subject to have recurrence of a disease/condition (e.g., cancer). In some cases, the methylation profile may be used to determine whether the subject has or subject to have non-recurrence of a disease/condition (e.g., cancer). In some cases, a methylation profile can comprise analysis (e.g., comprising sequencing) of a plurality of nucleic acids (e.g., a plurality of nucleic acid molecules of a depleted sequencing library, as described herein). In some cases, a methylation profile can comprise detection of methylated nucleotides and/or quantification of methylated nucleotide counts. In some cases, a methylation profile can comprise quantification of circulating tumor DNA (ctDNA). In some cases, the ctDNA can be quantified overtime (e.g., ctDNA kinetics). In some cases, a methylation profile can comprise determination of a methylated signal, e.g., in a population of nucleic acids of a depleted sequencing library, as described herein. In some cases, a methylation profile is compared to a genome-wide background profile. In some cases, a methylation profile is compared to a novel background profile created using hypermethylated cfDNA.

In some cases, a methylation profile can be analyzed to distinguish cancer cases from controls (e.g., non-cancer controls) with an area under the receiver operating characteristic curve (AUROC) of at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9%. In some cases, a methylation profile can be analyzed to distinguish cancer cases from controls (e.g., non-cancer controls) with an AUROC of at most about 90%, at most about 91%, at most about 92%, at most about 93%, at most about 94%, at most about 95%, at most about 96%, at most about 97%, at most about 98%, at most about 99%, at most about 99.1%, at most about 99.2%, at most about 99.3%, at most about 99.4%, at most about 99.5%, at most about 99.6%, at most about 99.7%, at most about 99.8%, or at most about 99.9%. In some cases, a methylation profile can be analyzed to distinguish cancer cases from controls (e.g., non-cancer controls) with an AUROC of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9%.

### Genomic Mutation Profile

The present disclosure provides methods, systems, and kits for producing a mutation profile of a subject that has a disease/condition or is suspected of having such disease/condition, wherein the mutation profile may be used to determine whether the subject has the disease/condition or is at risk of having the disease/condition. The samples disclosed herein can be subjected to library preparation and next generation deep sequencing, for example to a depth of 1 million (M) to 60 M single reads, 10 M to 60 M single reads, 10 M to 100 M single reads, 40 M to 60 M single reads, 40 M to 100 M single reads, 60 M to 100 M single reads, 60 M to 200 M single reads, 1 M to 10 M single reads, 1 M to 40 M single reads, 1 M single reads to 100 M single reads, 1 M single reads to 200 M single reads, at least 1 M single reads, at least 10 M single reads, at least 40 M single reads, at least 60 M single reads, at least 100 M single reads, or at least 200 M single reads. In some cases, sequencing can be performed at low sequencing depth (e.g., 10 M single reads, 20 M single reads, 30 M single reads, 40 M single reads, from 1 M single reads to 10 M single reads, from 10 M single reads to 20 M single reads, from 20 M single reads to 30 M single reads, from 30 M single reads to 40 M single reads, at most 10 M single reads, at most 20 M single reads, at most 30 M single reads, or at most 40 M single reads). In some cases, a sample disclosed herein can be subjected to 1 sequencing at a depth of 0.1× to 100×, 0.1× to 60×, 0.1× to 40×, 0.1× to 30×, 0.1× to 20×, 0.1× to 10×, 0.1× to 5.0×, 0.5× to 100×, 0.5× to 60×, 0.5× to 40×, 0.5× to 30×, 0.5× to 20×, 0.5× to 10×, 0.5× to 5.0×, 1.0× to 100×, 1.0× to 60×, 1.0× to 40×, 1.0× to 30×, 1.0× to 20×, 1.0× to 10×, 1.0× to 5.0×, at least 0.1×, at least 0.5×, at least 1.0×, at least 2.0×, at least 3.0×, at least 4.0×, at least 5.0×, at least 10.0×, at least 20.0×, at least 30.0×, at least 40.0×, at least 50.0×, at least 60.0×, at least 100×, at least 200×, at most 0.1×, at most 0.5×, at most 1.0×, at most 2.0×, at most 3.0×, at most 4.0×, at most 5.0×, at most 10.0×, at most 20.0×, at most 30.0×, at most 40.0×, at most 50.0×, at most 60.0×, at most 100×, or at most 200×. A plurality of sequencing reads is generated and analyzed. In some embodiments, deep sequencing may be configured to maximize identifying genomic mutations associated with the disease/condition.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. In some cases, the MAF of a ctDNA fraction of a sample can be about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5% 4%, 4.5% 5%, 5.5% 6%, 6.5%, 7%, 7.5% 8%, 8.5%, 9%, 9.5% 10%, or any percentage in between.

In some embodiments, a generated mutation profile of a subject can be generated from sequencing results. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range. The present disclosure provides methods, systems, and kits for producing a mutation profile of a subject that has a disease/condition or is suspected of having such disease/condition, wherein the methylation profile may be used to determine whether the subject has the disease/condition or is at risk of having the disease/condition. Producing a genomic mutation profile can comprise subjecting a plurality of nucleic acid molecules to library preparation and next generation deep sequencing (e.g., MeDIP-seq). A plurality of sequencing reads can be generated and analyzed, and, in some cases, deep sequencing may be configured to maximize identifying genomic mutations associated with the disease/condition. For example, a panel of canonical cancer driver genes may be included in a selector for sequencing results analysis. In some embodiments, including genes without documented driver effects in a particular cancer type in the analysis of sequencing data may increase the sensitivity of ctDNA detection.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. The ctDNA fraction of a sample disclosed herein is about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, the generated mutation profile of a subject does not include mutation variants derived from cell-free nucleic acid molecules derived from a biological sample. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range.

### Fragment Length Profile

In some embodiment, the length of ctDNA fragments is shorter than cell-free nucleic acid molecules derived from a healthy subject. In some embodiments, the length of ctDNA comprising at least one mutation is shorter than the length of cell free nucleic acid molecule containing a corresponding reference allele.

In some embodiments, the sequencing does not utilize bisulfite sequence because it causes degradation of ctDNA fragments and prevents the preservation of the length distribution of ctDNAs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be from 1 to about 800 basepairs (bp), from about 50 bp to about 800 bp, from about 100 bp to about 200 bp, from about 120 bp to about 150 bp, from about 60 to about 500 bp, from about 80 to about 300 bp, from 90 to about 250 bp, from 80 to 170 bp, or from about 100 to about 150 bp. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at least 800 basepairs (bp), at least 700 basepairs, at least 600 basepairs, at least 500 basepairs, at least 400 basepairs, at least 300 basepairs, at least 200 basepairs, at least 150 basepairs, at least 100 basepairs, or at least 50 basepairs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at most 800 basepairs (bp), at most 700 basepairs, at most 600 basepairs, at most 500 basepairs, at most 400 basepairs, at most 300 basepairs, at most 200 basepairs, at most 150 basepairs, at most 100 basepairs, or at most 50 basepairs. In some embodiments, the present disclosure provides an enrichment of the cell free nucleic acid samples based on selecting cell free molecules of a certain size. In some embodiments, the multimodal analysis comprises utilizing the mutation profile described herein and the fragment length profile by selectively including a plurality of nucleic acid molecules in the mutation profile based on their fragment length. In some embodiments, the multimodal analysis comprises utilizing the methylation profile described herein and the fragment length profile by selectively including a plurality of nucleic acid molecules in the methylation profile based on their fragment length. In some embodiments, the multimodal analysis comprises utilizing the mutation profile, methylation profile, and the fragment length profile together by selectively including a plurality of nucleic acid molecules in the mutation profile based on their fragment length and by selectively including a plurality of nucleic acid molecules in the methylation profile based on their fragment length respectively.

### Tumor Detection and Prognosis

The present disclosure provides methods and systems for determining whether a subject has or is at risk of having a disease, wherein the methods and systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, or any percentage in between the numbers. In some embodiments, the specificity is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, or any percentage in between the numbers. In some embodiments, the sensitivity when using two profiles is increased by at least about 0.5%, at least about 1%, at least about 2%, at least about 3%, at least about 4%, at least about 5%, at least about 6%, at least about 7%, at least about 8%, at least about 9%, at least about 10%, or percentage in between any of the numbers compared to the sensitivity when using one profile. In some embodiments, the sensitivity when using three profiles is increased by at least about 0.5%, at least about 1%, at least about 2%, at least about 3%, at least about 4%, at least about 5%, at least about 6%, at least about 7%, at least about 8%, at least about 9%, at least about 10%, or percentage in between any of the numbers compared to the sensitivity when using two profiles.

Further, the methods can provide a specificity of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, or any percentage in between the numbers. In some embodiments, the specificity when using two profiles is increased by at least about 0.5%, at least about 1%, at least about 2%, at least about 3%, at least about 4%, at least about 5%, at least about 6%, at least about 7%, at least about 8%, at least about 9%, at least about 10%, or percentage in between any of the numbers compared to the specificity when using one profile. In some embodiments, the specificity when using three profiles is increased by at least about 0.5%, at least about 1%, at least about 2%, at least about 3%, at least about 4%, at least about 5%, at least about 6%, at least about 7%, at least about 8%, at least about 9%, at least about 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, or any percentage in between the numbers. The methods provide a specificity of at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, or any percentage in between the numbers.

The present disclosure provides methods and systems for determining a tissue origin of a tumor, comprising identifying a nucleotide sequence specific for a particular cancer (e.g., breast cancer, colon cancer, prostate cancer, HSNCC, or lung cancer) from which a fraction of cell-free nucleic acid molecules. In some embodiments, the fraction of the cell-free nucleic acid molecules is derived from ctDNA. In some embodiments, the methods provide a sensitivity of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, or any percentage in between the numbers. The methods provide a specificity of at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, or any percentage in between the numbers.

The present disclosure provides methods and systems for determining whether a subject has or is at risk of having multiple diseases (e.g., multi-cancer), wherein the methods and systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said diseases at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, 9 at least about 9.7%, at least about 99.8%, at least about 99.9%, or any percentage in between the numbers. In some embodiments, the specificity is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, or any percentage in between the numbers. In some cases, method of determining whether a subject has or is at risk of having multiple diseases (e.g., multi-cancer) can comprise of determining 2, 3, 4, 5, or more diseases. In some cases, while determining whether a subject has or is at risk of having multiple diseases (e.g., multi-cancer), no disease, one disease (e.g., cancer) or more than one disease (e.g., multi-cancer) can be identified.

The present disclosure provides methods and systems for determining whether a subject has or is at risk of a cancer (e.g., low shedding cancer), wherein the methods and systems comprise (a) providing a plurality of nucleic acid molecules generated from a cfDNA sample of a subject, (b) subjecting said plurality of nucleic acid molecules or derivatives thereof to sequencing to generate a plurality of sequencing reads, (c) computer processing said plurality of sequencing reads generate a methylation profile for said plurality of nucleic acid molecules, and (d) computer processing said methylation profile to determine that said subject has cancer at an area under the receiver operating characteristic curve (AUROC) of at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9%. In some cases, the AUROC can be at most about 90%, at most about 91%, at most about 92%, at most about 93%, at most about 94%, at most about 95%, at most about 96%, at most about 97%, at most about 98%, at most about 99%, at most about 99.1%, at most about 99.2%, at most about 99.3%, at most about 99.4%, at most about 99.5%, at most about 99.6%, at most about 99.7%, at most about 99.8%, or at most about 99.9%. In some cases, the AUROC can be about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9%.

The present disclosure provides methods and systems for determining whether a subject has cancer (e.g., endometrial cancer, esophageal cancer, hepatobiliary cancer, prostate cancer, bladder cancer, breast cancer, colorectal cancer, head and neck cancer, lung cancer, pancreatic cancer, or renal cancer), wherein the methods and systems comprise (a) providing a plurality of nucleic acid molecules generated from a cell-free deoxynucleic acid (cfDNA) sample of a subject, (b) subjecting said plurality of nucleic acid molecules or derivatives thereof to sequencing to generate a plurality of sequencing reads in absence of bisulfite conversion, (c) computer processing said plurality of sequencing reads generate a methylation profile for said plurality of nucleic acid molecules, and (d) computer processing said methylation profile to determine that said subject has cancer.

The present disclosure provides methods and systems for determining whether a subject will experience recurrence of a cancer, wherein the methods and systems comprise (a) providing a plurality of nucleic acid molecules generated from a cell-free deoxynucleic acid (cfDNA) sample of a subject, (b) subjecting said plurality of nucleic acid molecules or derivatives thereof to sequencing to generate a plurality of sequencing reads in absence of bisulfite conversion, (c) computer processing said plurality of sequencing reads generate a methylation profile for said plurality of nucleic acid molecules, and (d) computer processing said methylation profile to determine that said subject has cancer.

The present disclosure provides methods and systems for determining whether a subject has or is at risk of having a disease or diseases, wherein the methods and systems comprise subjecting a plurality of nucleic acid molecules derived from a cell-free nucleic acid sample obtained from said subject to sequencing generate methylation profile, and comparing the sequences of the captured cell-free methylated DNA to control cell-free methylated DNAs sequences from healthy and cancerous individuals; detecting disease of the subject by determining statistically significant similarity between one or more sequences of the captured cell-free methylated DNA and cell-free methylated DNAs sequences from cancerous individuals.

In some embodiments, the control cell-free methylated DNAs sequences from healthy and cancerous individuals are comprised in a database of Differentially Methylated Regions (DMRs) between healthy and cancerous individuals.

The present disclosure describes methods and systems for providing a prognosis to a subject after receiving a treatment for a disease/condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based at least based on the at least one profile.

Once a subject is accurately diagnosed and receives a treatment to treat the cancer, such as surgical removal, chemotherapy, radio therapy, etc., it can be important to monitor the effectiveness of the treatment and predict the patient's survival rate. Further, it can be important to detect minimal residual disease of cancer cells.

In some embodiments, the method further comprises the operation of adding a second amount of control DNA to the sample for confirming the immunoprecipitation reaction.

As used herein, the “control” may comprise both positive and negative control, or at least a positive control.

In some embodiments, the method further comprises the operation of adding a second amount of control DNA to the sample for confirming the capture of cell-free methylated DNA.

In some embodiments, identifying the presence of DNA from cancer cells further includes identifying the cancer cell tissue of origin.

In some instances, tumor tissue sampling may be challenging or carry significant risks, in which case diagnosing and/or subtyping the cancer without the need for tumor tissue sampling may be desired. For example, lung tumor tissue sampling may require invasive procedures such as mediastinoscopy, thoracotomy, or percutaneous needle biopsy; these procedures may result in a need for hospitalization, chest tube, mechanical ventilation, antibiotics, or other medical interventions. Some individuals may not undergo the invasive procedures needed for tumor tissue sampling either because of medical comorbidities or due to preference. In some instances, the actual procedure for tumor tissue procurement may depend on the suspected cancer subtype. In other instances, cancer subtype may evolve over time within the same individual; serial assessment with invasive tumor tissue sampling procedures is often impractical and not well tolerated by patients. Thus, non-invasive cancer subtyping via blood test may have many advantageous applications in the practice of clinical oncology.

Accordingly, in some embodiments, identifying the cancer cell tissue of origin further includes identifying a cancer subtype. In some cases, the cancer subtype differentiates the cancer based on stage (e.g., early stage lung cancer treated with surgery vs late stage lung cancer treated with chemotherapy), histology (e.g., small cell carcinoma vs adenocarcinoma vs squamous cell carcinoma in lung cancer), gene expression pattern or transcription factor activity (e.g., ER status in breast cancer), copy number aberrations (e.g., HER2 status in breast cancer), specific rearrangements (e.g., FLT3 in AML), specific gene point mutational status (e.g., IDH gene point mutations), and DNA methylation patterns (e.g., MGMT gene promoter methylation in brain cancer).

In some embodiments, comparison can be based on fit using a statistical classifier. In some cases, the statistical classifier using DNA methylation data can be used for assigning a sample to multiple cancers. In some cases, the statistical classifier using DNA methylation data can be used for assigning a sample to multiple cancer for multi-cancer detection. In some cases, the multi-cancer detection can be multi-cancer early detection (MCED). In some cases, the statistical classifier using DNA methylation data can be used for assigning a sample to a minimal residual disease (MRD). In some cases, the statistical classifier using DNA methylation data can be used for assigning a sample to a particular disease state, such as cancer type or subtype (e.g., early-stage cancer, low shedding tumor). In some cases, the classifier can have differentially methylated regions from pairwise comparisons of each cancer type (or subtype) of interest when the classifier can distinguish multiple cancer types (or subtypes) from one another. In some cases, the statistical classifier can have one or more DNA methylation variables within a statistical model, and/or the output of the statistical model can have one or more threshold values to distinguish distinct disease states. In some cases, the statistical classifier can have feature(s) and/or threshold value(s) that can be derived from prior knowledge of the cancer types or subtypes, from prior knowledge of the features that are likely to be most informative, from machine learning, or from a combination of two of these approaches. In some embodiments, the classifier can be machine learning-derived. In some cases, the classifier can be an elastic net classifier, lasso, support vector machine, random forest, or neural network.

In some embodiments, comparisons can be carried out genome-wide. In other embodiments, the comparisons can be restricted from genome-wide to specific regulatory regions, such as, but not limited to, long interspersed nuclear elements (LINEs), short interspersed nuclear elements (SINEs), long terminal repeats (LTRs), FANTOM5 enhancers, CpG Islands, CpG shores, CpG Shelves, or any combination of the foregoing.

In some embodiments, the methods herein are for use in the detection of the cancer. In some embodiments, the methods herein are for use early cancer (e.g., early-stage cancer) detection. In some embodiments, the methods herein are for use in multi-cancer early (e.g., early-stage cancer) detection. In some embodiments, the methods herein are for use low shedding tumor detection. In some embodiments, the methods herein are for use in MRD detection. In some embodiments, the methods herein are for use low ctDNA detection.

In some embodiments, the methods herein are for use in monitoring therapy of the cancer.

### Data Analysis Systems and Methods

The methods and systems disclosed herein may comprise algorithms or uses thereof. The one or more algorithms may be used to classify one or more samples from one or more subjects. The one or more algorithms may be used to quantify ctDNA. The one or more algorithms may be used to predict treatment response. The one or more algorithms may be used to predict a disease/condition (e.g., cancer). The one or more algorithms may be applied to data from one or more samples. The data may comprise biomarker expression data. In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on at least one profile. The methods disclosed herein may comprise assigning a classification to one or more samples from one or more subjects. Assigning the classification to the sample may comprise applying an algorithm to the methylation profile, mutation profile, and fragment length profile. In some cases, at least one profile is inputted to a data analysis system comprising a trained algorithm for classifying the sample as obtained from a subject which has a disease or minor injuries.

A data analysis system may be a trained algorithm. The algorithm may comprise a linear classifier. In some instances, the linear classifier comprises one or more of linear discriminant analysis, Fisher's linear discriminant, Naïve Bayes classifier, Logistic regression, Perceptron, Support vector machine, or a combination thereof. The linear classifier may be a support vector machine (SVM) algorithm. The algorithm may comprise a two-way classifier. The two-way classifier may comprise one or more decision tree, random forest, Bayesian network, support vector machine, neural network, or logistic regression algorithms.

The algorithm may comprise one or more linear discriminant analysis (LDA), Basic perceptron, Elastic Net, logistic regression, (Kernel) Support Vector Machines (SVM), Diagonal Linear Discriminant Analysis (DLDA), Golub Classifier, Parzen-based, (kernel) Fisher Discriminant Classifier, k-nearest neighbor, Iterative RELIEF, Classification Tree, Maximum Likelihood Classifier, Random Forest, Nearest Centroid, Prediction Analysis of Microarrays (PAM), k-medians clustering, Fuzzy C-Means Clustering, Gaussian mixture models, graded response (GR), Gradient Boosting Method (GBM), Elastic-net logistic regression, logistic regression, or a combination thereof. The algorithm may comprise a Diagonal Linear Discriminant Analysis (DLDA) algorithm. The algorithm may comprise a Nearest Centroid algorithm. The algorithm may comprise a Random Forest algorithm. In some embodiments, for discrimination of preeclampsia and non-preeclampsia, the performance of logistic regression, random forest, and gradient boosting method (GBM) is superior to that of linear discriminant analysis (LDA), neural network, and support vector machine (SVM).

The present disclosure provides methods and systems for determining whether a subject has or is at risk of having a disease, wherein the methods and systems comprises subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profiles.

Further, the methods can provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods can provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease/condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on the at least one profile.

### Computer Systems

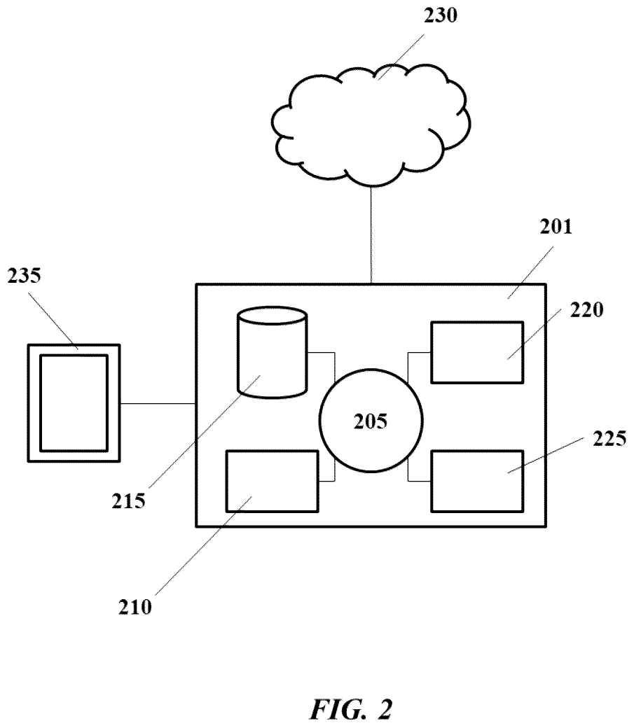

The present disclosure provides computer systems that are programmed to implement methods of the disclosure. FIG. 2 shows a computer system 201 that is programmed or otherwise configured to generate a sequencing library containing nucleic acid molecules that are depleted of hypermethylated regions of the nucleic acid molecules (e.g., ctDNA). The computer system 201 can regulate various aspects of the present disclosure. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries, and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

In some embodiments, the computer system can comprise a computer processing via a supervised machine learning method or an unsupervised machine learning method. In some cases, a supervised machine learning method can be a regression, support vector machine, tree-based method, neural network, or nearest neighbor method. In some cases, an unsupervised machine learning method can be clustering, neural network, principal component analysis, or matrix factorization.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 1135 that comprises a user interface (UI) 240. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Kits

The present disclosure provides kits for identifying or monitoring a disease or disorder (e.g., cancer) of a subject. A kit may comprise probes for identifying a quantitative measure (e.g., indicative of a presence, absence, or relative amount) of sequences at each of a panel of cancer-associated genomic loci in a sample of the subject. A quantitative measure (e.g., indicative of a presence, absence, or relative amount) of sequences at each of a panel of cancer-associated genomic loci in the sample may be indicative of the disease or disorder (e.g., cancer) of the subject. The probes may be selective for the sequences at the panel of cancer-associated genomic loci in the sample. A kit may comprise instructions for using the probes to process the sample to generate datasets indicative of a quantitative measure (e.g., indicative of a presence, absence, or relative amount) of sequences at each of the panel of cancer-associated genomic loci in a sample of the subject.

The probes in the kit may be selective for the sequences at the panel of cancer-associated genomic loci in the sample. The probes in the kit may be configured to selectively enrich nucleic acid (e.g., RNA or DNA) molecules corresponding to the panel of cancer-associated genomic loci. The probes in the kit may be nucleic acid primers. The probes in the kit may have sequence complementarity with one or more nucleic acid sequences from the panel of cancer-associated genomic loci or genomic regions. The panel of cancer-associated genomic loci or microbiome-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct panel of cancer-associated genomic loci or genomic regions.

The instructions in the kit may comprise instructions to assay the sample using the probes that are selective for the sequences at the panel of cancer-associated genomic loci in the cell-free biological sample. These probes may be nucleic acid molecules (e.g., RNA or DNA) having sequence complementarity with nucleic acid sequences (e.g., RNA or DNA) from one or more of the pluralities of panel of cancer-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of cancer-associated genomic loci in the sample. A quantitative measure (e.g., indicative of a presence, absence, or relative amount) of sequences at each of a panel of cancer-associated genomic loci in the sample may be indicative of a disease or disorder (e.g., cancer).

The instructions in the kit may comprise instructions to measure and interpret assay readouts, which may be quantified at one or more of the panels of cancer-associated genomic loci to generate the datasets indicative of a quantitative measure (e.g., indicative of a presence, absence, or relative amount) of sequences at each of the panel of cancer-associated genomic loci in the sample. For example, quantification of array hybridization or polymerase chain reaction (PCR) corresponding to the panel of cancer-associated genomic loci may generate the datasets indicative of a quantitative measure (e.g., indicative of a presence, absence, or relative amount) of sequences at each of the panel of cancer-associated genomic loci in the sample. Assay readouts may comprise quantitative PCR (qPCR) values, digital PCR (dPCR) values, digital droplet PCR (ddPCR) values, fluorescence values, etc., or normalized values thereof.

### Examples

### Example 1: Processing of Plasma-Derived Cell-Free DNA Using a Whole Methylome Enrichment Platform

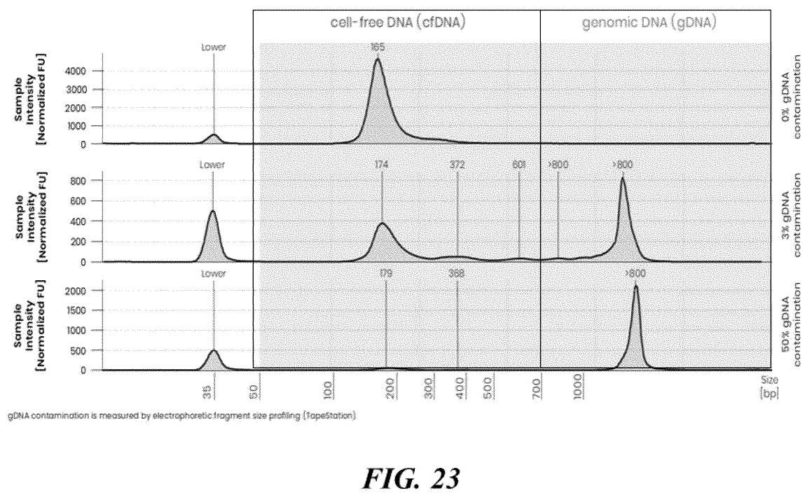

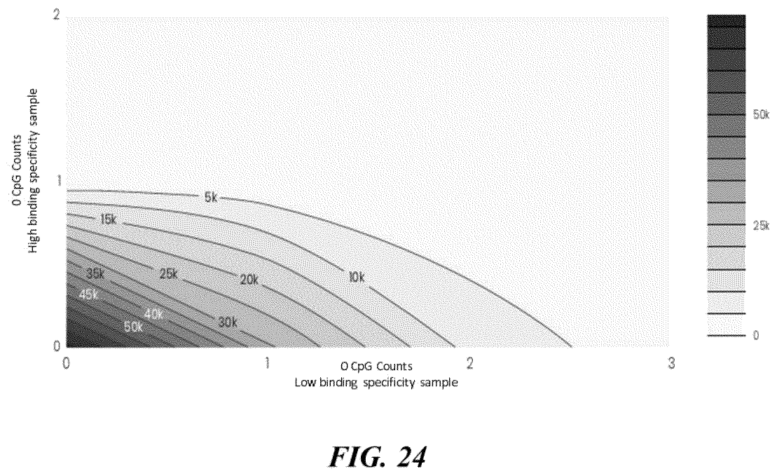

The cell-free methylated DNA immunoprecipitation and high-throughout sequencing (cfMeDIP-seq) methodology was developed as a non-degradative liquid biopsy approach to avoid the limitations of bisulfite-sequencing. Bisulfite-free approaches may require consistently high methylation binding specificity for the detection of potentially rare methylation events in circulating tumor DNA (ctDNA). Furthermore, as shown in FIG. 23, as genomic DNA contamination increased, there was reduced effective cfDNA input to the cfMeDIP-seq workflow, thereby reducing the probability to detect rare ctDNA, and emphasizing a need for an improved workflow. gDNA contamination was measured by electrophoretic fragment size profiling (e.g., TapeStation). There was no observed correlation between methylation specificity and percent of genomic DNA contamination. FIG. 24 also shows the importance of high methylation specificity for methylation-based liquid biopsy assays. The figure compares two clinical samples, one with high (y-axis) and one with low (x-axis) methylation specificity. 0 CpG counts indicate count of sequencing fragments aligning to a known 0 CpG region in the human genome. The gradient in the figure indicates the number of non-CpG regions in the genome with observed counts in each sample. Reads without CpGs and deviation of percent methylation specificity from 100% indicates non-specific binding by the anti-5mC antibody during immunoprecipitation. Perfect methylation specificity during immunoprecipitation (i.e., 100%) would result in non-CpG regions being void of DNA fragment counts. In real samples, generally the lower the methylation specificity the more counts are observed at regions without CpGs. Thus, in this example, cfMEDIP-seq was further refined with the aim of developing a robust genome-wide methylome enrichment platform for clinical use.

As shown in FIG. 25, four different cfMeDIP-seq workflows were conducted with samples of cell-free DNAs (cfDNAs) to determine the methylation binding specificity of each workflow. Samples included those derived from plasma of subjects as well as samples derived from genomic DNA that was sheared to mimic cfDNA. To conduct workflow 1, samples of cfDNAs (n=110) mixed with spike-in DNAs were subjected to library preparation. Next, filler DNAs were added to the prepped library to produce a sample mixture. The sample mixture was heat denatured and snap chilled. Then, 5-mc binders and magnetic beads were added sequentially to the mixture to incubate and allow the single-stranded DNA (ssDNA) to bind to the magnetic beads-5-mC binder complex. A magnet was used to isolate the magnetic bead-5mC binder complex and the captured ssDNAs was eluted off the complex. The captured ssDNAs underwent clean up before being subjected to PCR amplification for 14 cycles. Another round of clean up was performed with the PCR amplicons before quality control analysis, such as measuring methylation binding specificity.

Workflow 2 (n=174) was conducted similarly to workflow 1; however, an additional step of bead capturing was performed prior to adding filler DNAs. To perform bead capturing, elution post library preparation clean up was subjected to an additional capture against a magnetic rack, discarding any beads (e.g., solid phase reversible immobilization (SPRI) beads) carried over to minimize any nonspecific binding to the beads. Workflow 3 (n=63) was conducted the same as workflow 2, except the number of cycles used in the PCR amplification step was reduced to 13 cycles.

Workflow 4 (n=189) followed similar protocol as workflow 3, except a pre-binding step was performed, where the magnetic beads and 5-mC binders were separately mixed together to create magnetic bead-5-mC binder complexes. The magnetic bead-5-mC binder complexes were added after heat denaturing and snap chilling the mixture sample. Another difference between workflow 4 and workflow 3 was that instead of eluting the captured ssDNA from the magnetic bead-5-mC binder complexes and cleaning the captured ssDNA for PCR amplification, the PCR amplification step was performed on-bead.

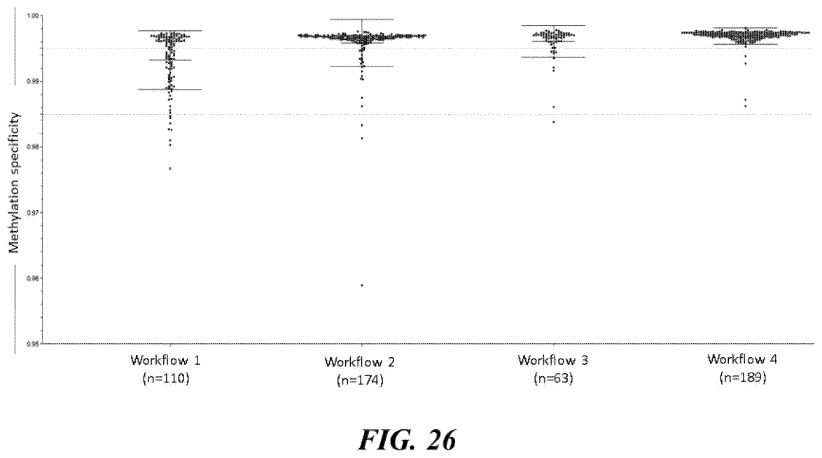

As shown in FIG. 26, for the different workflows, the mean methylation specificity was measured by calculating the percentage of synthetic oligonucleotide of known methylation status spiked into sample DNA at library preparation step recovered by immunoprecipitation (e.g., recovery of exogenous methylated fragments). Compared to the other workflows, workflow 4 resulted in greater methylation specificity and reduced variability among samples.

Methylation specificity of the enrichment platform were also analyzed on sample is different tubes and sample ages. Table 1 shows the impact that tube and sample age had on methylation specificity.

TABLE 1

| | | | |
|---|---|---|---|
| | | Methylation specificity, | |
| Experiment | N | mean (SD) | Delta |

Tube Type

| | | | |
|---|---|---|---|
| EDTA | 163 | 98.97 (1.08) | NA |
| Streck cfDNA | 37 | 99.28 (0.66) | 0.31% |

Sample Age (years)

| | | | |
|---|---|---|---|
| 0 to <5 | 107 | 99.00 (1.05) | NA |
| 5 to <10 | 65 | 99.13 (0.80) | 0.13% |
| ≥10 | 28 | 98.87 (1.33) | −0.13% |

The results showed that methylation specificity was consistent regardless of tube type (Δ=0.31%) or sample storage duration (Δ<0.26%)

### Example 2: Genome-Wide Methylome Enrichment Platform for Multi-Cancer Early Detection (MCED)

Genome wide mapping of DNA methylation in circulating cell-free DNA (cfDNA) can overcome critical sensitivity problem in detecting circulating tumor DNA (ctDNA) in subjects with early-stage cancer or low-shedding tumors.

To investigate the genome-wide methylome enrichment platform for multi-cancer early detection, a retrospective, case-control study was conducted with plasma samples from commercial and University Health Network biobanks. Samples were from individuals who had been diagnosed with cancer but had not yet begun treatment. For non-cancer controls, samples from age and sex-matched individuals who had no known cancer diagnosis were obtained. There was at least 12 months of confirmed cancer-free follow-up post-sample collection for the controls. Controls were excluded if, at the time of sample collection, the individual was 75 years or older, or they were known to have multiple comorbidities.

5-10 ng of cfDNA were extracted from plasma samples and were analyzed with bisulfite-free, non-degradative genome-wide DNA methylation enrichment platform, based on cell-free methylated DNA immunoprecipitation and high-throughput sequence (cfMeDIP-seq) technique, as described in Example 1, FIG. 25 (Workflow 1). Samples were split into distinct sets to train and test a machine learning classifier (e.g., linear based approach) made up of differentially methylated regions of whole methylome to distinguish cases from controls. Initial training in 1,536 samples across 8 cancer types and cross validation with 100 iterations of random splits (80:20) were performed to obtain area under the receiver operating characteristic curves (AUC) and 95% confidence intervals (CI) for the median probabilities. As shown in Table 2, all cancer cases were distinguished from controls with an AUC of 0.94 (95% CI: 0.93, 0.96), with AUCs for individual cancer types (e.g., bladder cancer, breast cancer, colorectal cancer, head and neck cancer, lung cancer, ovarian cancer, prostate cancer, renal cancer) ranging from 0.91 to 0.97. The AUC for all cancers in all stages was 0.94 (95% CI: 0.92, 0.95) for stage I/II cancers and 0.95 (95% CI: 0.94, 0.96) for stage II/IV cancers. The AUC was 0.92 (95% CI: 0.91, 0.94) in the subset of cancer that are low shedding (e.g., bladder cancer, breast cancer, prostate cancer, renal cancer), and had similar performance for stage I/II (AUC of 0.91; 95% CI: 0.89, 0.93), and stage III/IV (AUC of 0.93; 95% CI: 0.91, 0.95). These results suggest that the genome-wide methylome enrichment platform can detect multi-cancer at early stages and subset of cancer that are low shedding.

TABLE 2
Overall AUC (95%) for all cancers and by 8 cancer types.

| Cancer type | | All stages* | Stage I/II | Stage III/IV |
|---|---|---|---|---|
| All | N | 931 | 461 | 437 |
| Cancers | AUC (95% CI) | 0.94 (0.93, 0.96) | 0.94 (0.92, 0.95) | 0.95 (0.94, 0.96) |
| Bladder | N | 75 | 52 | 16 |
| Cancer** | AUC (95% CI) | 0.93 (0.91, 0.96) | 0.93 (0.90, 0.96) | 0.97 (0.95, 0.99) |
| Breast | N | 131 | 76 | 37 |
| Cancer** | AUC (95% CI) | 0.94 (0.91, 0.96) | 0.92 (0.88, 0.95) | 0.96 (0.94, 0.98) |
| Colorectal | N | 182 | 94 | 88 |
| Cancer | AUC (95% CI) | 0.97 (0.96, 0.98) | 0.97 (0.96, 0.98) | 0.96 (0.96, 0.99) |
| Head & Neck | N | 75 | 20 | 55 |
| Cancer | AUC (95% CI) | 0.96 (0.94, 0.98) | 0.93 (0.86, 1) | 0.97 (0.96, 0.99) |
| Lung | N | 147 | 72 | 75 |
| Cancer | AUC (95% CI) | 0.96 (0.95, 0.98) | 0.96 (0.95, 0.98) | 0.96 (0.95, 0.98) |
| Ovarian | N | 46 | 12 | 34 |
| Cancer | AUC (95% CI) | 0.96 (0.93, 0.98) | 0.98 (0.96, 0.99) | 0.95 (0.92, 0.98) |
| Prostate | N | 145 | 84 | 59 |
| Cancer** | AUC (95% CI) | 0.91 (0.88, 0.93) | 0.92 (0.89, 0.95) | 0.90 (0.87, 0.93) |
| Renal | N | 130 | 51 | 73 |
| Cancer** | AUC (95% CI) | 0.91 (0.89, 0.94) | 0.89 (0.84, 0.94) | 0.93 (0.89, 0.96) |

*Samples without stage information are included in this category
**Typically considered low-shedding tumor

Interim training in 1,906 samples across 12 cancer types (e.g., bladder cancer, breast cancer, colorectal cancer, endometrial cancer, esophageal cancer, head and neck cancer, hepatobiliary cancer, lung cancer, ovarian cancer, pancreatic cancer, prostate cancer, renal cancer) was also performed. Table 3 shows the characteristic between cancer and control cases. The machine learning classifier was used to distinguish cancer cases from non-cancer controls. Cross-validation with 20 iterations of 5-fold of the dataset was performed to obtain AUC and 95% CI for the median probabilities.

TABLE 3
Interim training cohort (N = 1,906)

| Characteristic | | Cancer Cases | Controls |
|---|---|---|---|
| Total | | 1,232 | 674 |
| Age | <50 years | 132 (10.7%) | 127 (18.8%) |
| | ≥50 years | 1,099 (89.2%) | 547 (81.2%) |
| | Unknown | 1 (0.1%) | 0 |
| Sex | Female | 485 (39.4%) | 303 (45%) |
| | Male | 747 (60.6%) | 371 (55.0%) |
| Smoking | Never Smoker | 589 (47.8%) | 571 (84.7%) |
| History | History of | 565 (45.9%) | 73 (10.8%) |
| | Smoking | | |
| | Unknown | 78 (6.3%) | 30 (4.5%) |

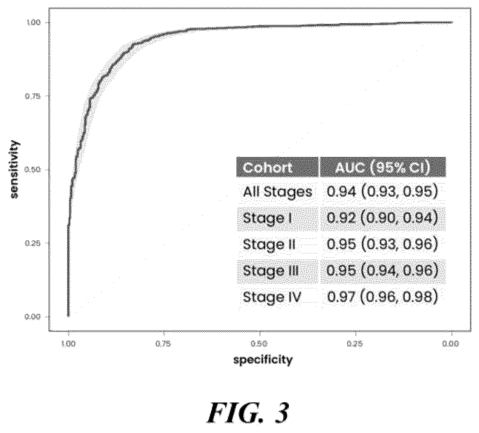

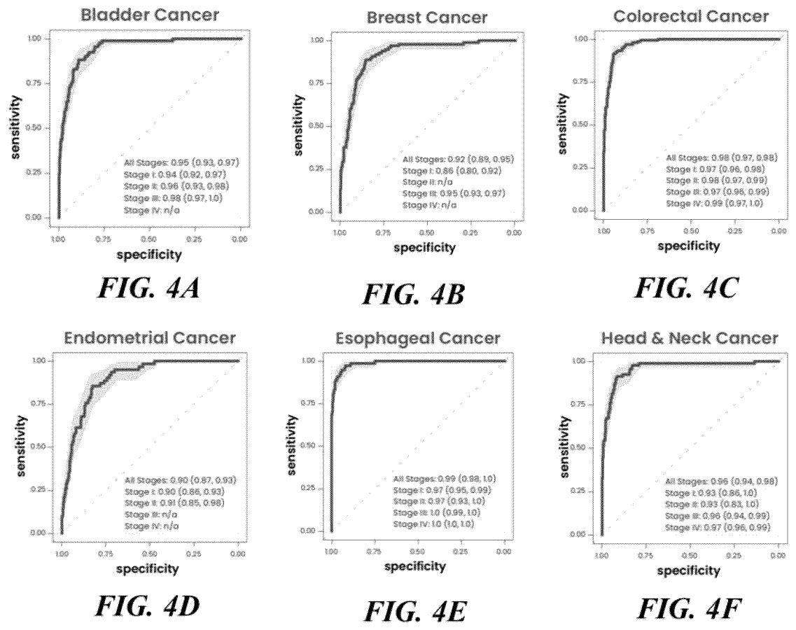

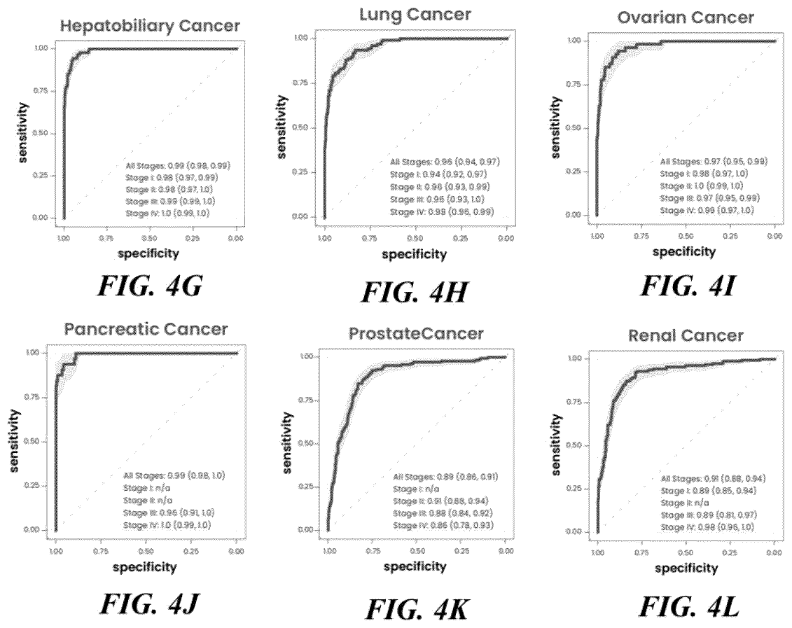

As shown FIG. 3, genome-wide methylation assay distinguished cancer cases from non-cancer controls with an overall AUC of 0.94 (95% CI: 0.93, 0.95). The AUCs by stage were 0.92 (stage I), 0.95 (stage II), 0.95 (stage III), and 0.97 (stage IV), showing that AUCs increased with the stages of cancer but remained high even for early-stage cancers. When each cancer type was evaluated separately (e.g., bladder cancer, breast cancer, colorectal cancer, endometrial cancer, esophageal cancer, head and neck cancer, hepatobiliary cancer, lung cancer, ovarian cancer, pancreatic cancer, prostate cancer, renal cancer), AUCs ranged from 0.89 to 0.99 as shown in FIGS. 4A-4L. The subset of cancers that are low shedding tumors (e.g., bladder cancer, breast cancer, endometrial cancer, and renal cancer) were also evaluated separately. The AUC of this subset of low shedding tumors was 0.91 (95% CI: 0.89, 0.93), thought only 8% were stage IV cancers, as shown in Table 4, suggesting that MCED can be possible with the genome-wide methylome enrichment platform.

TABLE 4
Stage Distribution in all cancers, 12 cancer types, and all low-shedding

| Cancer | All Stages¹ | Stage I | Stage II | Stage III | Stage IV |
|---|---|---|---|---|---|
| All Cancers | 1,232 | 284 (23%) | 280 (23%) | 327 (27%) | 221 (18%) |
| Bladder, N (%) | 94 | 42 (45%) | 19 (20%) | 16 (17%) | 4 (4%) |
| Breast, N (%) | 98 | 32 (33%) | 3 (3%) | 42 (43%) | 4 (4%) |
| Colorectal, N (%) | 169 | 18 (11%) | 75 (44%) | 44 (26%) | 32 (19%) |
| Endometrial, N (%) | 62 | 38 (61%) | 8 (13%) | 4 (6%) | 0 |
| Esophageal, N (%) | 77 | 9 (12%) | 16 (21%) | 34 (44%) | 18 (23%) |
| Head & Neck, N (%) | 92 | 7 (8%) | 17 (18%) | 23 (25%) | 44 (49%) |
| Hepatobiliary, N (%) | 88 | 24 (27%) | 18 (20%) | 23 (26%) | 13 (15%) |
| Lung, N (%) | 125 | 43 (34%) | 21 (17%) | 25 (20%) | 36 (29%) |
| Ovarian, N (%) | 54 | 6 (11%) | 7 (13%) | 32 (59%) | 6 (11%) |
| Pancreatic, N (%) | 32 | 0 | 4 (13%) | 5 (16%) | 23 (72%) |
| Prostate, N (%) | 183 | 1 (1%) | 90 (49%) | 55 (30%) | 25 (14%) |
| Renal, N (%) | 158 | 64 (41%) | 2 (1%) | 24 (15%) | 15 (9%) |
| All Low-Shedding², N (%) | 595 | 177 (30%) | 122 (21%) | 141 (24%) | 48 (8%) |

¹Includes cancers with unknown stage, incomplete staging information, and cancer recurrences.
²Includes cancer types that are traditionally considered low shedding tumors (bladder, breast, endometrial, prostate, and renal)

### Example 3: Evaluation of a Genome-Wide Methylome Enrichment Platform for ctDNA Quantification in Renal Cell Carcinoma (RCC)

Circulating tumor DNA (ctDNA) can be utilized to identify the presence of cancer as well as minimal residual disease. Quantification of ctDNA using plasma-based tests can be a useful cancer management tool to assess prognosis; however, some methodologies also require tumor tissue for analysis or are limited to tumor types that tend to have higher amounts of associated ctDNA. This experiment demonstrated the feasibility of using a tumor-uninformed genome-wide methylome enrichment platform to quantify ctDNA in plasma and predict prognosis in RCC.

Biobanked pre-treatment samples from individuals with newly diagnosed stage I-IV RCC (University Health Network, Ontario Tumor Biobank) were analyzed with a bisulfite-free, non-degradative genome-wide DNA methylation enrichment platform using 5-10 ng of cell-free DNA isolated from plasma, as described in Example 1, FIG. 25 (Workflow 1). ctDNA was quantified from average normalized counts across informative regions.

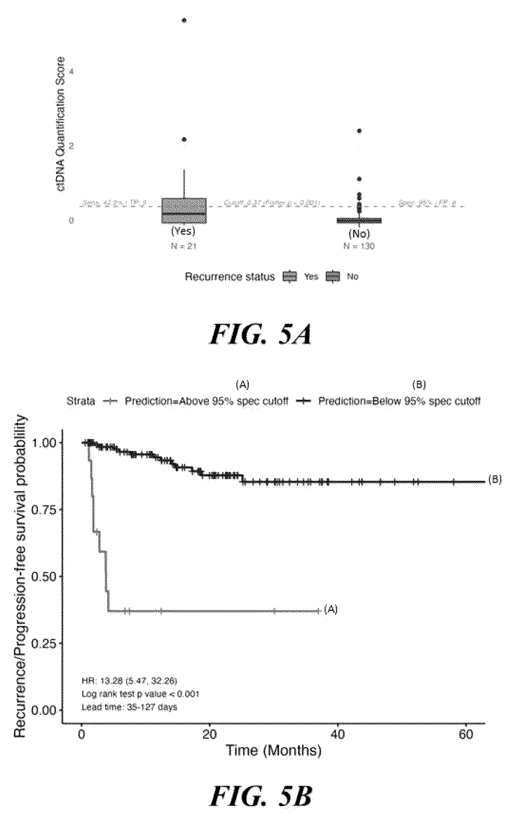

The algorithm used 1-150 base pair (bp) reduced-size fragments containing at least 10 CpGs to identify cancer-associated methylation. Counts were summarized across non-overlapping 300 bp windows and normalized by sequencing depth. A single factor (e.g., cancer vs. non-cancer controls) was used to contrast methylation signal across each 300 bp window. For each window, a p-value was calculated between cancers and non-cancer controls by Wilcoxin rank-sum test. The Benjamini-Hochberg method was used to adjust the p-value. Significant differentially methylated regions were selected using an adjusted p-value threshold less than or equal to 0.1. Differential methylation analysis identified 2027 hypermethylated regions enriched for CpG islands. ctDNA quantification scores were generated based on average normalized counts across the 2027 regions and adjusted for methylation specificity. Events were defined as cancer recurrence or progression. A ctDNA quantity threshold was set such that 95% of samples without an event fell below the threshold (e.g., 95% specificity). Time to event was compared for samples with ctDNA quantities above the threshold to those below the threshold. FIG. 5A shows that the threshold for renal cancer was set at 0.37.

The cohort included 151 samples [64 stage I, 2 stage II, 23 stage III, 15 stage IV, 47 with unknown or incomplete stage information]. There was a median follow-up of 15.7 months and a total of 21 events. Samples with ctDNA quantities above the threshold were significantly more likely to recur or progress than those below the threshold [hazard ratio 13.28 (95% CI 5.47, 32.26), log-rank P<0.001] (FIG. 5B). Samples with ctDNA quantities below the threshold were more than likely to avoid recurring or relapsing of the cancer.

This experiment demonstrated the feasibility of using a blood-based, tumor-uninformed genome-wide methylome enrichment platform for ctDNA quantification and prognostic prediction in renal cancer. This is a promising demonstration of prognostic performance in a cancer type that is typically difficult to detect due to low amounts of ctDNA. Additional evaluation in post-treatment and longitudinal samples will further test this platform.

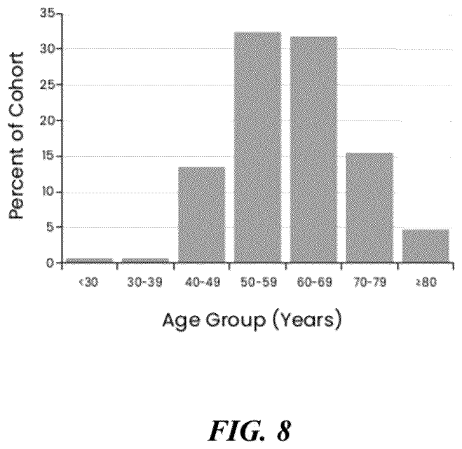

In another similar study, biobanked plasma samples from individuals with newly diagnosed stage I-IV RCC (collected from 2015 to 2021; Princess Margaret Cancer Centre at University Health Network and the Ontario Tumour Bank) were included. All samples were obtained after cancer diagnosis but prior to surgery or other definitive treatment. Table 5 shows the clinic-pathologic information of the 148 samples used, and FIG. 8 shows the age of the individuals at the time of sample collection.

TABLE 5
Clinico-Pathologic Information (N = 148)

| Characteristic | | N (%) |
|---|---|---|
| Sex | Female | 42 (28%) |
| | Male | 106 (72%) |
| Stage | Stage I | 64 (43%) |
| | Stage II | 2 (1%) |
| | Stage III | 23 (16%) |
| | Stage IV | 15 (10%) |
| | Late Stage (III/IV) | 39 (26%) |
| | Unknown | 5 (3%) |
| Histology | Clear Cell Carcinoma | 112 (76%) |
| | Chromaphobe RCC | 13 (9%) |
| | Papillary Carcinoma | 12 (8%) |
| | Other | 11 (7%) |
| Smoking | Yes | 81 (55%) |
| History | No | 64 (43%) |
| | Unknown | 3 (2%) |

All samples were analyzed with a bisulfite-free, non-degradative genome-wide DNA methylation enrichment platform, using about 5-10 ng of cfDNA extracted from plasma. The genome-wide methylation assay used was based on the cell-free methylated DNA immunoprecipitation and high-throughput sequencing (cfMeDIP-seq) technique, as described in Example 1, FIG. 25 (Workflow 1).

For analysis, an event was defined as cancer recurrence, progression, or death due to renal cancer (whichever occurred earliest). A ctDNA quantity threshold for baseline prognostication was set such that about 95% of samples without an event fell below the threshold (i.e., about 95% specificity). Event-free survival was estimated using the Kaplan-Meier method, and the difference was assessed by the log-rank test using both the total population and individuals with stages I-III cancers.

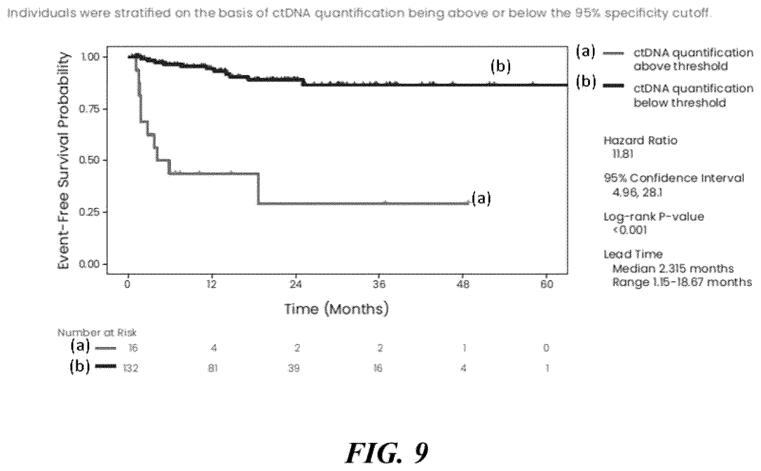

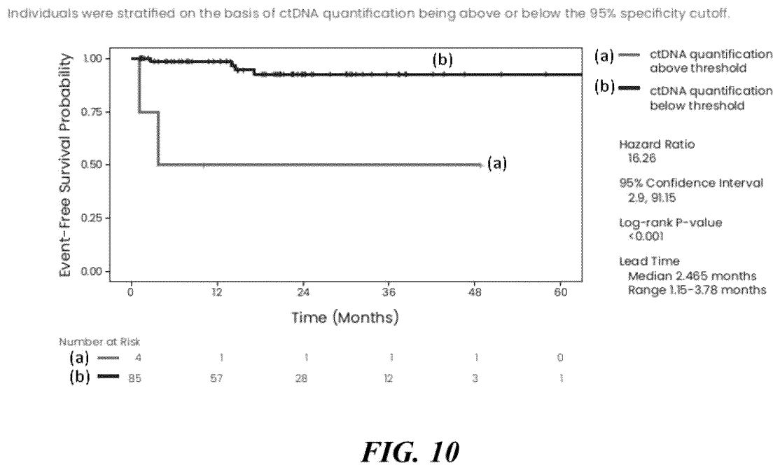

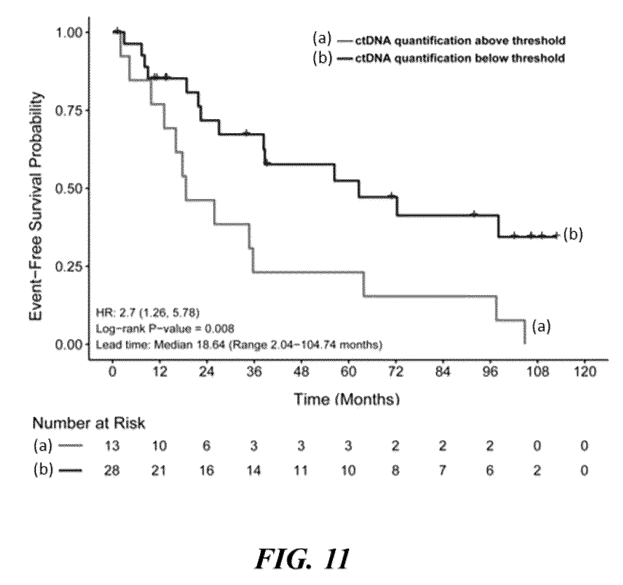

As shown in FIG. 9, individuals with ctDNA quantification above the cutoff displayed significantly worse event-free survival in the overall cohort. Furthermore, as shown in FIG. 10, individuals with ctDNA quantification above the cutoff also displayed significantly worse event-free survival in the sub-population with stage I-III disease. Individuals were stratified on the basis of ctDNA quantification being above or below the 95% specificity cutoff.

Thus, these data demonstrated the feasibility of using a blood-based genome-wide methylome enrichment platform for ctDNA quantification and determining prognostic performance in RCC. The performance observed also represents a promising demonstration of prognostication in a cancer type that is typically difficult to detect due to low amounts of ctDNA. In addition, the assay utilized here is tumor-naïve, meaning that patient-specific tumor tissue is not required to generate a bespoke panel for ctDNA.

### Example 4: Prognostic Performance of a Genome-Wide Methylome Enrichment Platform in Head and Neck Cancer

The utilization of plasma-based tests to quantify circulating tumor DNA (ctDNA) is emerging as a promising new approach to cancer management. ctDNA quantification can be used to assess prognosis and to detect minimal residual disease following initial treatment. This experiment demonstrated the feasibility of using a tumor-uninformed genome-wide methylome enrichment platform for ctDNA quantification and prognostic prediction in head and neck cancer.

Biobanked pre-treatment samples from individuals with newly diagnosed stage I-IV head and neck cancer (University Health Network) were analyzed with a bisulfite-free, non-degradative genome-wide DNA methylation enrichment platform using 5-10 ng of cell-free DNA isolated from plasma, as described in Example 1, FIG. 25 (Workflow 4). ctDNA was quantified from average normalized counts across informative regions.

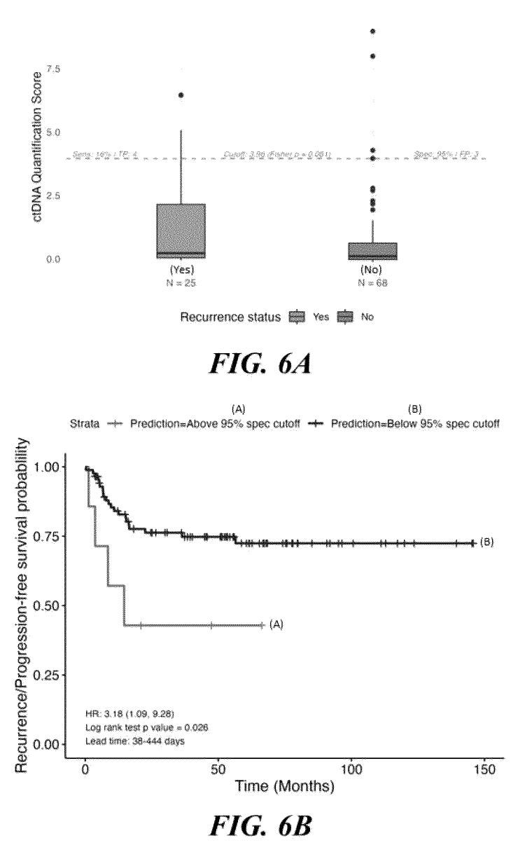

The algorithm used 1-150 bp reduced-size fragments containing at least 10 CpGs to identify cancer-associated methylation. Counts were summarized across non-overlapping 300 bp windows and normalized by sequencing depth. A single factor (e.g., cancer vs. non-cancer controls) was used to contrast methylation signal across each 300 bp window. For each window, a p-value was calculated between cancers and non-cancer controls by Wilcoxin rank-sum test. The Benjamini-Hochberg method was used to adjust the p-value. Significant differentially methylated regions were selected using an adjusted p-value threshold less than or equal to 0.1. Differential methylation analysis identified 2027 hypermethylated regions enriched for CpG islands. ctDNA quantification scores were generated based on average normalized counts across the 2027 regions and adjusted for methylation specificity. Events were defined as cancer recurrence or progression. A ctDNA quantity threshold was set such that 95% of samples without an event fell below the threshold (e.g., 95% specificity). Time to recurrence or progression event was compared for samples with ctDNA quantities above the threshold to those below the threshold. FIG. 6A shows that the threshold for head and neck cancers was set at 3.96.

Among 93 samples included (7 stage I, 17 stage II, 23 stage III, 46 stage IV), the median follow-up time was 50.6 months, with 25 events. The likelihood of recurrence or progression was significantly higher in samples with ctDNA above the threshold [hazard ratio (HR) 3.18 (95% CI 1.09, 9.28), log-rank P=0.026] (FIG. 6B). In multivariate analysis accounting for cancer stage and clinical characteristics (sex, age, smoking history, BMI), ctDNA quantification above the threshold showed a similar association [HR 3.51 (95% CI 1.1, 11.19), P=0.034]. This experiment demonstrated the feasibility of using a blood-based, tumor-uninformed genome-wide methylome enrichment platform for ctDNA quantification and prognostic prediction in head and neck cancer, using treatment-naïve plasma samples.

Furthermore, the utilization of plasma-based tests to quantify ctDNA can also be used to assess prognosis and improve post-treatment surveillance. For head and neck cancer, this offers an opportunity to inform whether chemotherapy should be administered after surgery in some non-metastatic tumors (Stage I-IVb) and improve post-treatment surveillance by detecting minimal residual disease ahead of clinical detection.

In another similar study, plasma samples from individuals with newly diagnosed stage I-IV head and neck cancer (collected from 2008 to 2019; Princess Margaret Cancer Centre, University Health Network) were included. All samples were obtained after cancer diagnosis but prior to surgery or other definitive treatment. All samples were analyzed with a bisulfite-free, non-degradative genome-wide DNA methylation enrichment platform using 5-10 ng of cell-free DNA isolated from plasma. This assay was based on the cell-free methylated DNA immunoprecipitation and high-throughput sequencing (cfMeDIP-seq) technique, as described in Example 1, FIG. 25 (Workflow 4). ctDNA was quantified using a machine-learning algorithm across informative regions.

For analysis, events were defined as cancer recurrence, progression, or death due to head and neck cancer (whichever occurred earliest). The maximum follow-up time was 5 years after diagnosis. A ctDNA quantity threshold was set such that 95% of samples without an event fell below the threshold (i.e., 95% specificity). Event-free survival was estimated using Kaplan-Meier method, and compared samples with ctDNA above the threshold to those below the threshold. The difference between the two groups was assessed by the log-rank test. Multivariable Cox regression analysis was used to adjust for known prognostic covariates.

Table 6 shows the clinic-pathologic information of the subjects. Table 7 shows the overview of primary cancer treatment of the subjects. As shown in Tables 6 and 7, a total of 91 samples were included and the mean follow-up time was 50.6 months, with 27 events.

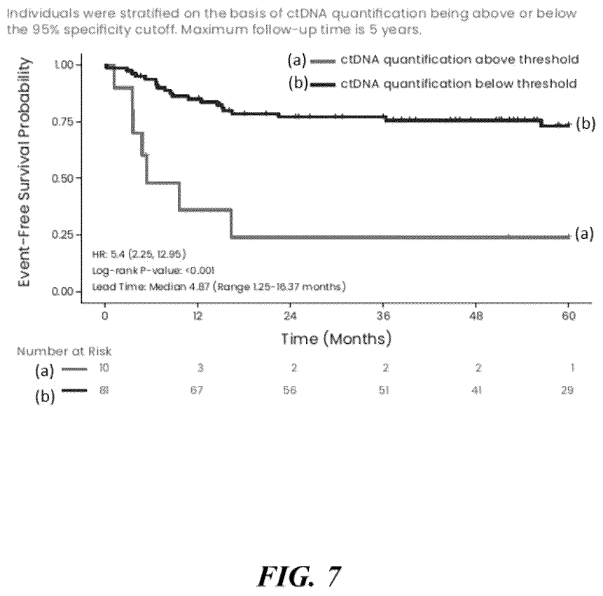

Individuals with ctDNA levels above the threshold displayed significant worse event-free survival, as shown on FIG. 7. The lead time (time interval from blood draw to event) ranged from 1.25 to 16.37 months, a median lead time of 4.87 months (FIG. 7). In multivariate analysis according for cancer stage and clinical characteristics, ctDNA quantification above the threshold showed a similar association (Table 8).

TABLE 6
Clinico-Pathologic Information (N = 91)

| Characteristic | Mean [SD] |
|---|---|
| Age at Sample Collection | 60.99 [11.03] |

| Characteristic | | N (%) |
|---|---|---|
| Sex | Female | 14 (15%) |
| | Male | 77 (85%) |
| Stage (AJCC | Stage I | 7 (8%) |
| 7th Edition) | Stage II | 16 (18%) |
| | Stage III | 23 (25%) |
| | Stage IV | 45 (50%) |
| Cancer Site | Oropharynx | 56 (62%) |
| and HPV Status | HPV+ | 39 (70%) |
| | HPV− | 17 (30%) |
| | Lip & Oral Cavity | 15 (17%) |
| | Larynx | 16 (18%) |
| | Hypopharynx | 4 (4%) |
| Smoking | Yes | 62 (68%) |
| History | No | 29 (32%) |

TABLE 7
Overview of Primary Cancer Treatment

| | HPV+ | HPV− | Lip & | | Hypo- |
|---|---|---|---|---|---|
| Treatment | Oropharynx | Oropharynx | Oral Cavity | Larynx | pharynx |
| Surgery Only | 2 (5%) | 0 (0%) | 5 (33%) | 1 (6%) | 0 (0%) |
| Surgery + | 2 (5%) | 0 (0%) | 6 (40%) | 2 (13%) | 0 (0%) |
| Radiation | | | | | |
| Surgery + | 1 (3%) | 1 (6%) | 2 (13%) | 0 (0%) | 0 (0%) |
| Chemoradiation | | | | | |
| Radiation Only | 17 (44%) | 11 (65%) | 1 (7%) | 12 (75%) | 3 (75%) |
| Chemoradiation | 17 (44%) | 4 (24%) | 1 (7%) | 1 (6%) | 1 (25%) |
| Only | | | | | |
| No Treatment | 0 (0%) | 1 (6%) | 0 (0%) | 0 (0%) | 0 (0%) |

TABLE 8
Multivariate Analysis

| | | | |
|---|---|---|---|
| | Hazard | 95% Confidence | p- |
| Variable | Ratio (HR) | Interval (CI) | value |

| | | | | |
|---|---|---|---|---|
| ctDNA | Below | Ref | — | — |
| Quantification | Threshold | | | |
| | Above | 5.24 | 2.05, 13.42 | 0.001 |
| | Threshold | | | |
| Stage | Early (I/II) | Ref | — | — |
| | Late (III/IV) | 1.19 | 0.41, 3.46 | 0.744 |
| Age | <60 years | Ref | — | — |
| | ≥60 years | 1.15 | 0.52, 2.52 | 0.725 |
| Sex | Female | Ref | — | — |
| | Male | 0.77 | 0.25, 2.39 | 0.647 |
| Smoking | No | Ref | — | — |
| History | Yes | 1.3 | 0.54, 3.13 | 0.557 |

Similar to the previous experiment, this experiment also demonstrated the feasibility of using a blood-based, tumor-uninformed genome-wide methylome enrichment platform for ctDNA quantification and prognostic prediction in head and neck cancer. The test utilized tumor-naïve, which means that the patient-specific tumor tissue is not required to generate a bespoke panel for ctDNA detection. Tumor uninformed approaches may allow for more flexible clinical use by 1) not requiring access to original tumor tissue, and 2) not being limited to the genomic regions that were differentially methylated at diagnosis, which may allow for sensitive longitudinal minimal residual disease (MRD) detection.

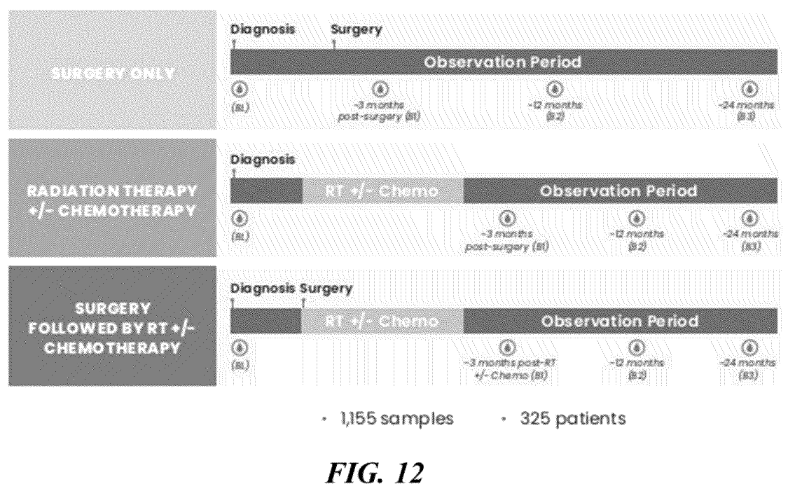

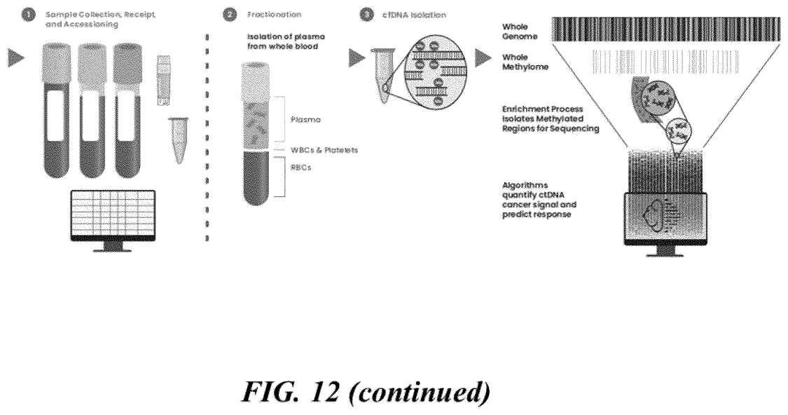

Tissue-agnostic, genome-wide methylome enrichment platform based on cfMEDIP-seq in head and neck cancer can also be used to predict recurrence for purposes of guiding adjuvant therapy after completion of curative-intent treatment and to detect early relapse. To investigate detection of MRD in head and neck cancer patients after curative intent treatment, biobanked samples from individuals with stage I-IVb human papillomavirus (HPV)-negative and HPV-positive head and neck cancer with longitudinal data collection and sampling (Princess Margaret Cancer Centre) were analyzed. The full cohort included 325 unique patients with 1,155 samples. Patients diagnosed with Epstein-Barr virus-associated nasopharyngeal carcinoma were excluded. Out of the full cohort, 1,119 of 1,155 (96.90%) samples passed the quality threshold for cfDNA quantity and processed through cfMeDTP. The samples were split into distinct sets to train and test a machine learning classifier with differentially methylated regions. As shown in FIG. 12, the blood collection time points included at diagnosis, prior to curative intent therapy (baseline (BL)), and approximately 3 (landmark timepoint, B1), 12 (B2) and 24 (B3) months after curative intent treatment. Curative intent treatment included surgery alone, radiotherapy (RT)+/−chemotherapy, or surgery+radiotherapy+/−chemotherapy. 5-10 ng of plasma cfDNA for each sample was used and subjected to the bisulfite-free, non-degradative genome-wide methylome enrichment platform based on cfMeDIP-seq, as further detailed in Example 1, FIG. 25 (Workflow 4). MRD signals were quantified from average normalized counts across informative methylated regions and binarized into a positive and negative group. Recurrence-free survival (RFS) was compared for patients tested positive to those tested negative at 3 months post curative treatment and longitudinally.

A total of 173 samples from 52 unique patients (stage I (33%), stage II (17%), stage III (23%), IV (27%)) were analyzed and correlated with recurrence in this interim training result. At the landmark timepoint, patients who tested positive showed significantly worse RFS than those tested negative (Hazard ratio (HR) 8.91; 95% CI, 3.14-25.26, P<0.001). Incorporating serial longitudinal samples, RFS continued being statistically significantly worse in patients tested positive than those tested negative (HR 10.47; 95% CI, 3.81-28.82, P<0.001).

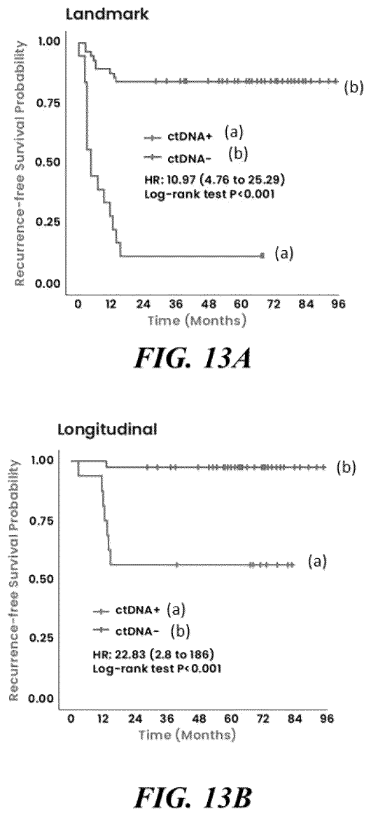

Similarly, a larger total sample of 249 samples collected from 75 patients were also analyzed. The clinic pathologic information of the 75 patients is shown in Table 9. The analysis included comparing the prognosis of ctDNA detected and ctDNA not detected cancer patients. The Kaplan-Meier (KM) method was used to compare the RFS between the two groups using the two-sided log-rank test. The HR between the groups was estimated using a cox proportional hazards model. These analyses were performed at the landmark timepoint (FIG. 13A) and longitudinally (FIG. 13B). As shown in FIGS. 13A and 13B, ctDNA positivity was found to be predictive of survival outcomes in post-curative intent treated patients with head and neck cancer. ctDNA positivity correlated with RFS at the landmark timepoint and longitudinally. Significant differences in RFS were observed with a HR of 10.97 (CI: 4.76 to 25.29; p-value<0.001) at the landmark timepoint and a HR of 22.83 (CI: 2.8 to 186); p-value<0.001) longitudinally when patients were stratified by ctDNA status. These interim training analyses demonstrated that MRD detection with a blood-based, tissue-agnostic, genome-wide methylome enrichment platform in HNC patients after curative intent treatment has a strong correlation with RFS with hazard ratios consistent with tumor-informed assays.

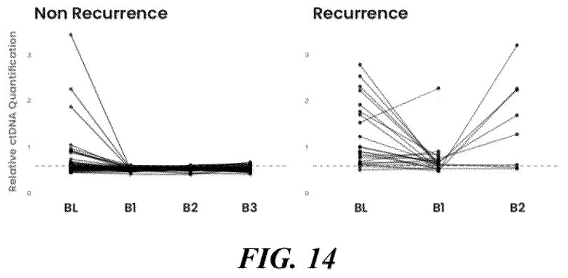

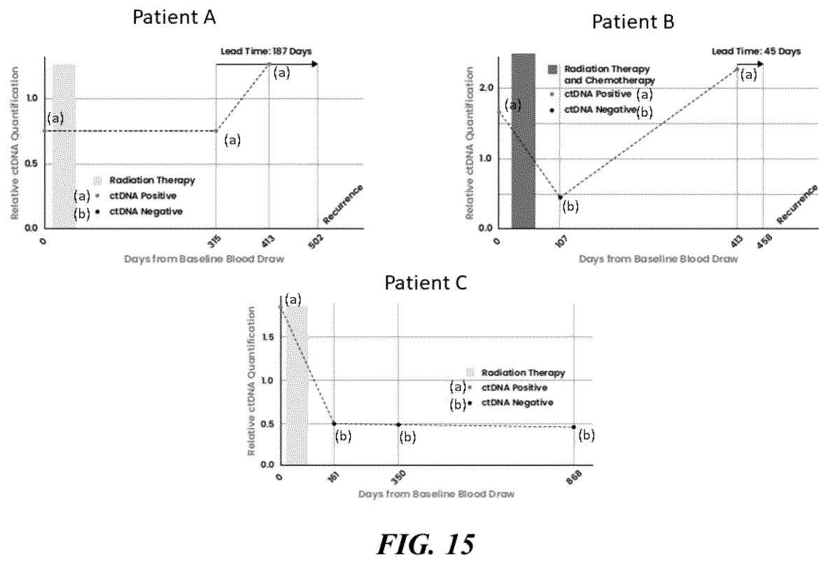

Further, the genome-wide methylome enrichment platform based on cfMEDIP-seq was found to be capable of monitoring ctDNA kinetics, which is the quantitative changes in ctDNA levels over time. ctDNA was quantified and plotted over time for all subjects who recur and subjects who do not have a recurrence even to demonstrate the ability to monitor ctDNA kinetics. As shown in FIG. 14, ctDNA quantification trajectory plot demonstrated correlation with outcomes of non-recurrence and recurrence. Estimated ctDNA quantification from pre-treatment (BL) to post-treatment (e.g., B1, B2, B3) was consistent with the expected ctDNA kinetics. ctDNA for individual patients before and after curative intent treatment was also analyzed. FIG. 15 shows representative case studies of ctDNA kinetics in three individual patients (Patient A, Patient B, Patient C). Patient A (FIG. 15, top left) was diagnosed with a Stage III, HPV− tumor of the oropharynx. ctDNA was detected at diagnosis prior to treatment with RT and at 315 and 413 days after diagnosis preceding detection of a distant recurrence at 502 days (lead time of 187 days). Patient B (FIG. 15, top right) was diagnosed with a Stage IVA HPV+ tumor of the hypopharynx. ctDNA was detected at diagnosis prior to treatment with chemoradiation (CRT) but not detected at 107 days after diagnosis and after completion of CRT. ctDNA was detected again 413 days after diagnosis preceding detection of a distant recurrence at 458 days (lead time of 45 days). Patient C (FIG. 15, bottom) was presented with Stage I HPV+ tumor of oropharynx. ctDNA was detected at diagnosis prior to radiation therapy (RT) but was not detected at any post treatment timepoint. Patient C remained disease free until the last clinical follow up (>2.5 years). These data showed that blood-based tissue agnostic genome-wide methylome enrichment platform can demonstrate robust performance for ctDNA quantification and monitoring ctDNA kinetics in HPV+ and HPV− head and neck cancers.

Collectively, these results may aid in treatment escalation or de-escalation decisions after curative intent treatment and be used to monitor for recurrence before clinical or radiographic presentation.

TABLE 9
Clinico-demographic information of patients
Overall (N = 75) Recurrence (N = 25) Non-Recurrence (N = 50)

| Characteristic | Mean [SD] |
|---|---|
| Age | 62.35 [9.5] |

| Characteristic | | N (%) |
|---|---|---|
| Sex | Female | 18 (24%) |
| | Male | 57 (76%) |
| Stage (AJCC | Stage I | 26 (35%) |
| 8th Edition) | Stage II | 12 (16%) |
| | Stage III | 16 (21%) |
| | Stage IV | 21 (28%) |
| Histology | Squamous cell | 72 (96%) |
| | carcinoma (SCC) | |
| | Non-SCC | 3 (4%) |
| Site and | Oropharynx | 39 (52%) |
| HPV Status | HPV+ | 31 (79%) |
| | Lip & Oral | 20 (27%) |
| | Cavity | |
| | Larynx | 10 (13%) |
| | Other | 6 (8%) |
| Smoking | Yes | 42 (56%) |
| History | No | 25 (33%) |
| | Unknown | 8 (11%) |

### Example 5: Pre-Analytic Variables and Quality Controls for Robust Processing of Plasma-Derived Cell-Free DNA (cfDNA) Using a Whole Methylome Enrichment Platform

The cell-free methylated DNA immunoprecipitation and high-throughput sequencing (cfMeDIP-seq) methodology was developed as a non-degradative liquid biopsy approach to avoid the limitations of bisulfite-sequencing. However, bisulfite-free approaches may need consistently high methylation binding specificity for the detection of potentially rare methylation events in circulating tumor DNA (ctDNA). cfMeDIP-seq has been refined with the aim of developing a robust genome-wide methylome enrichment platform for clinical use. In this experiment, the impact of pre-analytic variables and analytic configuration on the methylation specificity (e.g., pulled-down methylated region specificity) of the platform was investigated.

cfDNA from plasma was used for the genome-wide methylome enrichment platform. cfDNA was subjected to standard library preparation, combined with DNA filler, denatured, and subjected to immunoprecipitation using an anti-5-mC antibody. Captured DNA, enriched for methylation, was amplified and sequenced, as described in Example 1, FIG. 25 (Workflow 4). Methylation specificity (e.g., pulled-down methylated region specificity) of the immunoprecipitation step was monitored using methylated and unmethylated spike-in DNA fragments added prior to adapter ligation. Methylation specificity was evaluated across several pre-analytic variables: sample collection tube type (Streck, EDTA), sample age (0-5 years, 5-10 years, ≥10 years), and genomic DNA (gDNA) contamination (1%-50% gDNA). Each was evaluated as the difference between the mean methylation specificity (categorical variables) or a correlation (continuous variables) in a cohort of >4,000 archival plasma samples from individuals with and without cancer. The immunoprecipitation step was further optimized to increase centrality and reduce variability of methylation specificity using 20 replicates from donor-derived cfDNA.

Methylation specificity (e.g., pulled-down methylated region specificity) was consistent regardless of tube type (Δ=0.29%) or sample age (Δ<0.32%). There was no correlation between methylation specificity when comparing samples with gDNA contamination ranging from 1%-50% (Kendall's correlation=0.0064). Modifications to the immunoprecipitation step were evaluated to improve methylation specificity, with significant improvement observed post optimization (Kolmogorov-Smirnov p-value<0.00001). The optimized assay had a mean methylation specificity of 99.7%, with 20/20 samples displaying ≥99.6% specificity.

The data indicated that the genome-wide methylome enrichment platform is a robust and versatile test, with minimal impact from pre-analytic variables and consistently high methylation specificity.

### Example 6: Analytical Performance of a Genome-Wide Methylome Enrichment Platform to Detect Minimal Residual Disease from Plasma-Derived Cell-Free DNA

Tissue-agnostic approaches for detecting cancer signals from plasma may have significant benefit, especially where tissue is not accessible for evaluation or where speed to answer matters. Plasma-derived cell-free DNA (cfDNA) can be used to detect cancer, including minimal residual disease (MRD) in patients who have undergone curative cancer treatments. However, these tests may need highly sensitive methods of cancer signal detection for clinical application. Thus, a genome-wide methylome enrichment platform that uses cell-free methylated DNA immunoprecipitation and high-throughput sequencing (cfMeDTP-seq) methodology can be combined with custom algorithms that leverage differentially methylated regions (DMRs) found in cfDNA to distinguish between cancer and non-cancer signals. Thus, analytical performance metrics of an algorithm in development for detecting MRD using the methylation-based approached was investigated.

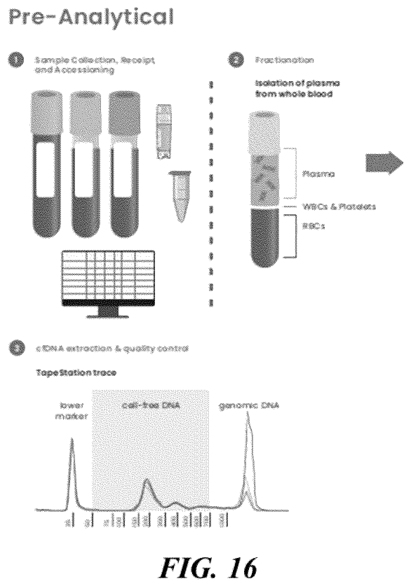

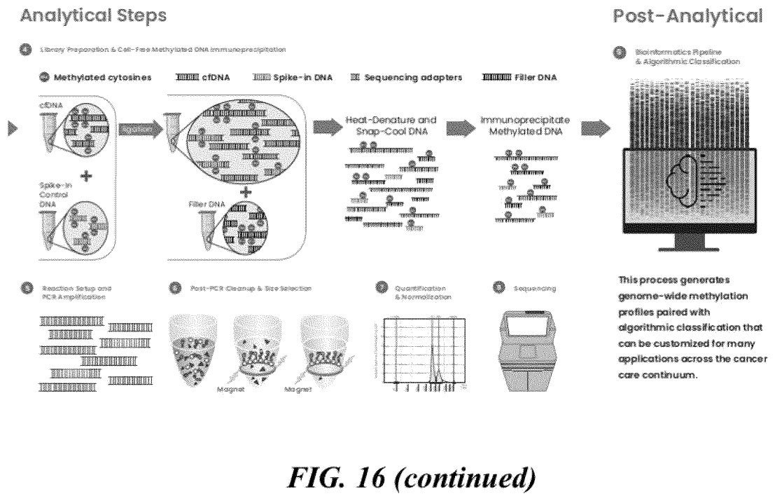

Plasma-derived cfDNAs were used for the genome-wide methylome enrichment platform. As shown in FIG. 16, and further detailed in Example 1 (FIG. 25—Workflow 4), plasma-derived cfDNAs (10 ng input mass) combined with spike-in DNA fragments were subjected to standard library preparation, combined with DNA filler, denatured, and subjected to immunoprecipitation using an anti-5-mC antibody. Captured DNA, enriched for methylation, was amplified, and sequenced. The sequencing results were subjected to bioinformatics pipeline and algorithmic classification. A candidate algorithm comprised of DMRs was used to quantify cancer-specific methylation. Limit of detection and precision were then evaluated using contrived cancer samples intended to mimic low-level circulating tumor DNA (ctDNA) representative of MRD.

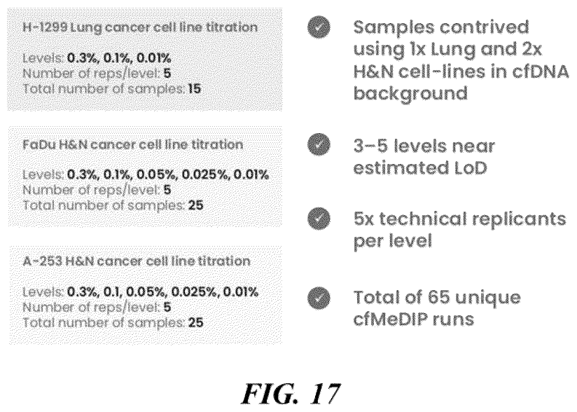

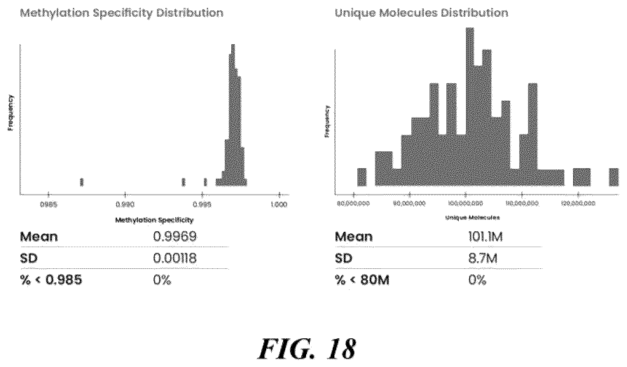

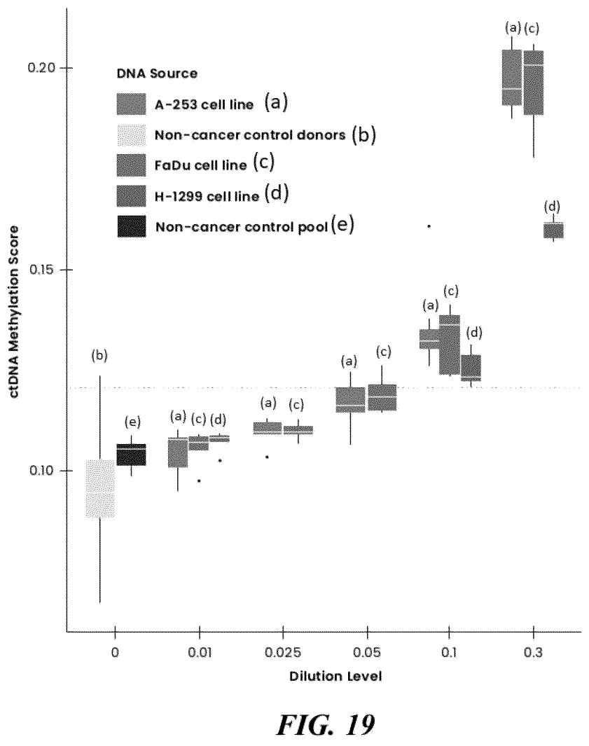

To evaluate limit of detection, samples were contrived by titrating enzymatically fragmented DNA from three immortalized tumor-derived cell-lines (H-1299 lung cancer cell line, FaDu head and neck (H&N) cancer cell line, A-253 H&N cancer cell line) into a pooled non-cancer donor-derived cfDNA in a titration series targeting less than 1% ctDNA levels. As outlined in FIG. 17, five technical replicants were used per level of ctDNA, totaling 65 unique cfMeDIP runs. As shown in FIG. 18, all non-cancer and contrived cancer samples met in-process quality control metrics, which included greater than or equal to 98.5% methylation specificity and greater than or equal to 80 million unique molecules. Further, as shown in FIG. 19, the ctDNA methylation score was measured for the different level of ctDNA, and the limit of detection calculation at 95% sensitivity (LoD95) was found to be less than 0.1% using the tissue-agnostic algorithm. The threshold was set using 12 non-cancer donors and 5 replicates of a non-cancer pool to establish a 95% true negative rate.

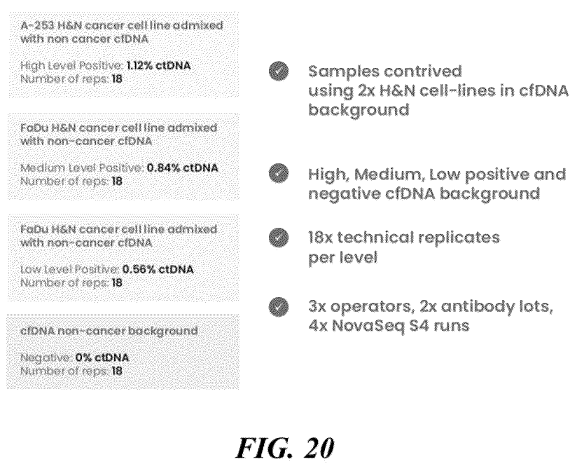

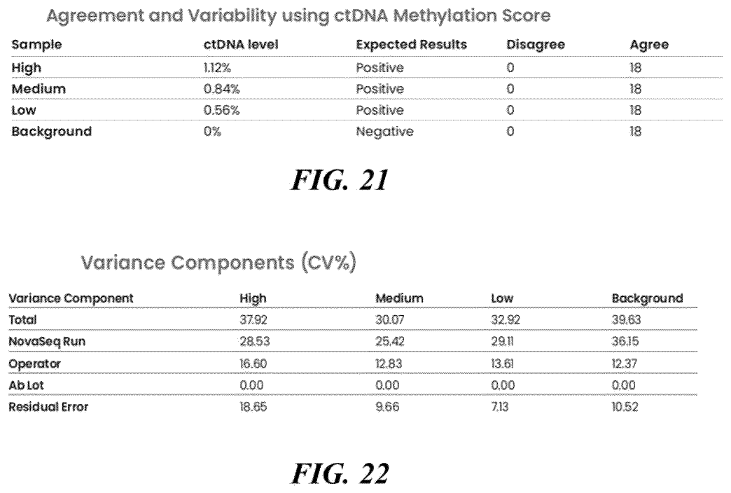

To evaluate precision of the blood-based tissue agnostic genome-wide methylome enrichment platform, samples were contrived by titrating enzymatically fragmented DNA from two immortalized tumor-derived cell lines (A-253 H&N cancer cell line, FaDu H&N cancer cell line) into pooled non-cancer donor-derived cfDNA at high (1.12%), medium (0.84%), and low (0.56%) ctDNA levels in 18 technical replicates, as outlined in FIG. 20. Next, the samples were process with the blood-based tissue agnostic genome-wide methylome enrichment platform in replication with varying operators, sequencing runs and antibody reagent lots. As shown in FIG. 21, using the ctDNA methylation score, the agreement to the expected results and variability was able to be assessed. In all levels of ctDNA, the outcome agreed with the expected results. Further, as shown in FIG. 22, the combination of the total variable analytics (operators, sequencing runs and antibody reagent lots) led to a variance component (CV %) of less than 40% for all levels of ctDNA.

Collectively, the evaluation of the limit of detection and precision demonstrated the use of a blood-based tissue-agnostic genome-wide methylome enrichment platform utilizing non-degradative methodology combined with specific algorithms and DMRs at level appropriate for MRD detection.

### Example 7: Prognostic Performance of a Genome-Wide Methylome Enrichment Platform in Early-Stage Non-Small Cell Lung Cancer (NSCLC)

Circulating tumor DNA (ctDNA) can be utilized to identify the presence of cancer as well as minimal residual disease (MRD). Quantification of ctDNA can be a useful cancer management tool to assess prognosis. In this example, the feasibility of using a tumor-naïve genome-wide methylome enrichment platform to quantify ctDNA in plasma and predict recurrence in early-stage non-small cell lung cancer (NSCLC) was evaluated.

In a retrospective evaluation of banked, pre-treatment samples from newly diagnosed Stage I and II NSCLC patients (collected from 2009 to 2013, Princess Margaret Cancer Centre) were used. The blood samples were obtained after cancer diagnosis and before treatment. Samples were analyzed with a bisulfite-free, non-degradative genome-wide DNA methylation enrichment platform using 5-10 ng of cell-free DNA isolated from plasma, as described in Example 1, FIG. 25 (Workflow 4). Samples from 41 patients were included. Table 10 describes the clinico-demographic information of the patients. ctDNA was quantified from average normalized counts across informative regions. Events were defined as cancer recurrence or death due to any causes, whichever occurred earlier. A ctDNA quantity threshold was set where 100% of samples from patients without an event fell below the threshold (i.e., 100% specificity). Time to recurrence or death was compared for samples with ctDNA quantities above the threshold to those below the threshold. Recurrence-free survival was estimated using Kaplan-Meier method and compared for samples with ctDNA quantities above versus below the threshold. The difference between the two groups was assessed by the log-rank test. Multivariable Cox regression analysis was used to adjust for known prognostic value of pre-treatment ctDNA level, adjusted for covariates identified in univariate analyses.

There were 27 events, with a median follow-up time of 55.8 months. Samples with ctDNA above the threshold showed significantly worse recurrence-free survival as shown in FIG. 11, [hazard ratio (HR) 2.70 (95% CI 1.26, 5.78), log-rank P=0.008]. Multivariant analysis (Table 11) showed that samples with ctDNA above the threshold showed significantly worse recurrence-free survival even after accounting for histology (selected using univariate analysis) [HR 2.79 (95% CI 1.30, 6.02), P=0.009].

Collectively, the data showed that this blood-based genome-wide methylome enrichment platform can be used for ctDNA quantification and prognostication in early-stage NSCLC. Initial feasibility was evaluated here using treatment-naïve plasma samples. Applications for cancer management will be further evaluated in future studies utilizing post-treatment and longitudinal samples.

TABLE 10
Clinico-demographic information of patients

| | | Overall |
|---|---|---|
| Clinical Characteristics | | (n = 41) |
| Age | Mean (SD); years | 70.9 (11.25) |
| | Median (Q1, Q3) | 74 (66, 78) |
| | Min, Max | 39, 91 |
| Sex, | Female | 22 (54%) |
| n (%) | Male | 19 (46%) |
| Smoking | Current | 4 (10%) |
| Status, | Former | 27 (66%) |
| n (%) | Never | 10 (24%) |
| Body | Normal | 12 (29%) |
| Mass | Overweight | 16 (39%) |
| Index | Obese | 8 (20%) |
| (BMI) | Unknown | 5 (12%) |
| Category, | | |
| n (%) | | |
| Stage, | Stage IA | 20 (49%) |
| n (%) | Stage IB | 7 (17%) |
| | Stage IIA | 10 (24%) |
| | Stage IIB | 4 (10%) |
| Histology, | Non-Small Cell | 27 (66%) |
| n (%) | Adenocarcinoma | |
| | Non-Small Cell | 14 (34%) |
| | Squamous Cell | |
| | carcinoma | |

TABLE 11
Multivariate Analysis

| | | | 95% | |
|---|---|---|---|---|
| | | Hazard | Confidence | P- |
| Variables | | Ratio (HR) | Interval (CI) | value |
| ctDNA | Below Threshold | Reference | | |
| Quant | Above Threshold | 2.79 | 1.30, 6.02 | .009 |
| Histology | Non-Small | Reference | | |
| | Cell Adenocarcinoma | | | |
| | Non-Small | 2.07 | 0.95, 4.49 | .067 |
| | Cell Squamous cell | | | |

### Example 8: Cancer Methylome Versus Whole Methylome

Various amounts of nucleosomal cell-free DNA (ncfDNA) derived from FaDu cancer cell line were generated to mimic cancer signal and were diluted into a background of cfDNA pooled from multiple cancer-free donors. Table 12 shows the dilution points and technical replications, totaling 32 samples, that were created to be used for limit of detection (LoD) assessment.

TABLE 12
Samples with different dilution points and technical replicates

| Dilution | | | | | |
|---|---|---|---|---|---|
| percentage | 0% | 0.02% | 0.05% | 0.1% | 0.3% |
| Technical | 6 | 8 | 6 | 6 | 6 |
| replicates | | | | | |

10 ng of input DNA from each of the 32 samples were subjected to genome-wide DNA methylation enrichment platform, based on cell-free methylated DNA immunoprecipitation, as described in Example 1, FIG. 25 (Workflow 4), to produced enriched libraries. After performing the cell-free methylated DNA immunoprecipitation, the enriched libraries were mixed with probes to target regions of interest, as shown in FIG. 28. Two target capture pools were created by evenly dividing enriched libraries generated from each dilution percentage (Table 13), ensuring equal number of technical replicates for each dilution percentage in each pool, for a total of 16 samples per pool. Each pool underwent a capture process with set of probes targeting the following cancer methylome target panels, followed by Twist Target Enrichment Standard Hybridization v2 protocol to generated enriched libraries for sequencing on the Illumina next-generation sequencing system. The cancer methylome target panels were made up with the following: 1) housekeeping methylated regions that are methylated across different samples regardless of cancer status, 2) regions without CpGs, used to calculate endogenous binding specificity scores for quantifying non-specific methylation binding as a quality control measure, 3) cancer hyper differentially methylated regions (DMRs), 4) noise regions, 5) regions with low counts in cancer-free samples (controls).

TABLE 13
Enriched libraries divided into two target capture pools

| | | | | | | Input |
|---|---|---|---|---|---|---|
| | | | | | | DNA per |
| Dilution | | | | | | sample |
| percentage | 0% | 0.02% | 0.05% | 0.1% | 0.3% | (ng) |
| Samples | 3 | 4 | 3 | 3 | 3 | 93.75 |
| in capture | | | | | | |
| pool 1 | | | | | | |
| Samples | 3 | 4 | 3 | 3 | 3 | 93.75 |
| in capture | | | | | | |
| pool 2 | | | | | | |

Target enriched samples were sequenced using Illumina NovaSeq 6000 sequencer aiming at approximately 26 million reads per sample. Next, the sequencing reads from target captured samples were aligned to hg38 human reference genome using Bowtie2 alignment tool. A unique molecular identifier (UMI) based approach was employed to identify and remove sequence duplications that may have occurred by PCR or flow cell optical clustering error. DNA sequences were further filtered by size, minimal number of CpGs and mapping quality before being counted in targeted genomic regions of interest (ROI).

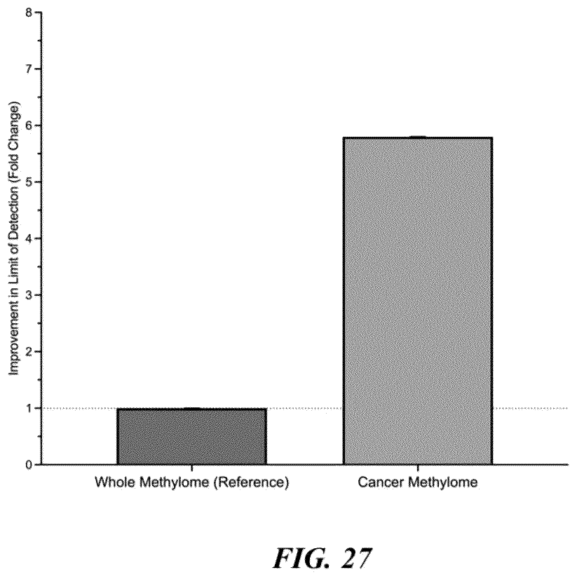

Quantification of the cancer DNA was performed using counts of DNA fragments in the targeted ROIs and generating a score by comparing to an internal baseline. Scores derived from the same ROIs were used to compare the cancer methylome approach of integrating the additional enrichment step with probes versus the whole methylome approach, which does not integrate the addition capturing of target regions with probes. The whole methylome result was based on prior cell line titration study and the top performing scores The same regions were used to get scores for the cancer methylome. Next, the fold change was calculated by dividing whole methylome approach LoD by cancer methylome approach LoD. Quantification of ctDNA across different dilution points was used to determine the LoD in both approaches. As shown in FIG. 27, there was improvement in the LoD in the cancer methylome approach versus the whole methylome approach, as presented as fold change, showing that integrating the additional enrichment step with probes can improve the LoD.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

1-184. (canceled)

185. A method for detecting minimal residual disease (MRD), comprising:
(a) assaying a biological sample from a subject, wherein said assaying does not comprise analyzing a solid tumor sample of the subject, wherein said assaying comprises sequencing of one or more enriched methylated regions in said biological sample; and
(b) detecting said MRD at a specificity of at least 90% or at a sensitivity of at least 80% based at least in part on said sequencing of said one or more methylated regions.

186. The method of claim 185, wherein said biological sample is a plasma or blood sample.

187. The method of claim 185, wherein said biological sample comprises a plurality of methylated nucleic acid molecules.

188. The method of claim 185, wherein said biological sample has no more than 50 ng of nucleic acid molecules.

189. The method of claim 187, wherein said assaying comprises enriching for said plurality of methylated nucleic acid molecules using a capture reagent, thereby generating a plurality of enriched nucleic acid molecules.

190. The method of claim 189, wherein said enriching is under conditions sufficient to increase a fold enrichment ratio associated with said plurality of methylated nucleic acid molecules.

191. The method of claim 189, wherein said capture reagent comprises a binder.

192. The method of claim 191, wherein said binder comprises a Methyl-CpG-binding domain.

193. The method of claim 188, wherein said enriching comprises immunoprecipitating said plurality of methylated nucleic acid molecules using an antibody.

194. The method of claim 189, further comprising contacting said plurality of enriched nucleic acids with one or more nucleic acid capture probes to enrich for one or more target sequences.

195. The method of claim 194, wherein said one or more nucleic acid capture probes comprises sequences associated with healthy samples.

196. The method of claim 187, further comprising amplifying said plurality of methylated nucleic acids to generate a plurality of amplified nucleic acids.

197. The method of claim 185, wherein said sequencing of said one or more methylated regions comprises unbiased sequencing or targeted sequencing.

198. The method of claim 185, comprising detecting said MRD at a specificity of at least 95% or at a sensitivity of at least 90%.

199. The method of claim 185, comprising detecting said MRD at an AUROC of at least about 90%.

200. The method of claim 185, further comprising processing sequencing reads of said one or more methylated regions to generate a methylation profile of said biological sample.

201. The method of claim 200, wherein said processing comprises using a machine-learning derived classifier.

202. The method of claim 201, wherein said machine-learning derived classifier comprises an elastic net classifier, lasso, support vector machine, random forest, or neural network.

203. The method of claim 200, wherein said methylation profile comprises whole methylome.

204. The method of claim 200, wherein said methylation profile comprises targeted methylome.
